(19) United States
(12) Patent Application Publication
(10) Pub. No.: US 2012/0088230 A1
(43) Pub. Date: Apr. 12, 2012

(54) System And Method For Cell Analysis

(76) Inventors: Monique Givens, Westminster, CO (US); Jeffrey Ives, Arvada, CO (US); Michael J. Lochhead, Boulder, CO (US); Marie J. Delaney, Boulder, CO (US); Kevin D. Moll, Boulder, CO (US); Keagan B. Rowley, Boulder, CO (US); Kurt R. Vogel, Boulder, CO (US); Christopher J. Myatt, Boulder, CO (US)

(21) Appl. No.: 13/270,973

(22) Filed: Oct. 11, 2011

Related U.S. Application Data
(60) Provisional application No. 61/391,909, filed on Oct. 11, 2010.
(60) Provisional application No. 61/475,189, filed on Apr. 13, 2011.
(60) Provisional application No. 61/479,268, filed on Apr. 26, 2011.

(51) Int. Cl. C12Q 1/70; C12Q 1/02; C12M 1/34; G01N 21/75

(52) U.S. Cl. 435/5; 435/7.1; 435/29; 435/288.7; 435/287.2

(57) ABSTRACT

A system for enumeration of objects such as cells in a sample is disclosed. The system uses a low-cost cartridge and a reader instrument, based on planar waveguide imaging technology. Cells of a blood sample may be stained with fluorescence-tagged antibodies and are loaded onto the cartridge where the differentially labeled cells may be distinguished and quantified.

### RELATED APPLICATIONS

This application claims priority to U.S. Provisional Patent Application No. 61/391,909, filed Oct. 11, 2010 and entitled “System for Fluorescence Microscopy with Autofocus.” This application also claims priority to U.S. Provisional Patent Application No. 61/475,189, filed Apr. 13, 2011 and entitled “System and Method for Enumerating Cells in a Sample.” This application also claims priority to U.S. Provisional Patent Application No. 61/479,268 filed Apr. 26, 2011 and entitled “System and Method for Cell Analysis.” All of the aforementioned applications are incorporated by reference into the present application in their entireties and for all purposes.

### GOVERNMENT INTEREST

This invention was made with Government support under the U.S. Department of Commerce National Institute of Standards (“NIST”) Advanced Technology Program (“ATP”), award number 70NANB7H7053, and National Institutes of Health (“NIH”) award number 2R44AI070052. The Government has certain rights in this invention.

### BACKGROUND

I. Field of the Invention

The present disclosure pertains to identification and enumeration of an analyte or an object in a sample. More particularly, the disclosure relates to a system and methods for enumerating specific cells such as CD4 T helper lymphocytes in a human blood sample.

II. Description of Related Art

The human immunodeficiency virus (“HIV”) is a retrovirus that infects cells of the immune system. After it gains access into an individual host, HIV may impair or eventually destroy the normal immune function of the infected individual. As the immune system becomes weaker, the infected individual becomes more susceptible to other infections. The most advanced stage of HIV infection is commonly known as Acquired Immunodeficiency Syndrome (“AIDS”). Over the last three decades or so, AIDS has spread globally and has become one of the biggest health challenges in many parts of the world, especially resource-limited settings.

HIV mediated CD4 cell destruction is the central immunologic feature of HIV infection. Thus, the CD4 count is a critical measurement in initial disease staging, in monitoring antiretroviral therapy and in managing primary and secondary prophylaxis for opportunistic infections. In fact, quantitative T helper cell counts in the range of 0 to 1000 cells per microliter are a critical indicator for initiating and optimizing anti-retroviral treatment and preventing viral drug resistance. Flow cytometry is the current standard-of-care for CD4 cell counting. Unfortunately, flow cytometry is a central lab-based technique; sample cold chain requirements as well as transport, equipment, and operational costs render the technique cost-prohibitive in limited resource settings where HIV prevalence is highest. A simple, point-of-care (“POC”) CD4 counting tool will fundamentally change HIV management.

### SUMMARY

The present instrumentalities advance the art by providing a simple diagnostic system that solves many of the problems in the field. In one embodiment, the system may include a device and a reader instrument. Examples of the device may include but are not limited to a cartridge, a substrate, a channel, or other solid supports capable of transmitting light and holding the sample. In another embodiment, the reader instrument may be capable of capturing images on the device and processing the image data.

In one embodiment, the device may contain a waveguide. In another embodiment, the device may contain a first substrate and a second substrate. In another embodiment, the first substrate or the second substrate may contain a planar waveguide. In another embodiment, the planar waveguide may be a multi-mode planar waveguide. In another embodiment, the planar waveguide may have an integrally formed lens. In another embodiment, the planar waveguide may contain at least a first outer surface and a first inner surface, while the second substrate may contain at least a second outer surface and a second inner surface. In another embodiment, the first inner surface and the second inner surface are spaced apart from each other, wherein the first inner surface and the second inner surface at least partly define a sample chamber that may hold or confine the entire sample or a portion thereof. In another embodiment, the first substrate and the second substrate are positioned such that at least a section of the first inner surface and a section of the second inner surface are apart from each other at a distance wherein this section of the first inner surface and this section of the second inner surface at least partly define a sample chamber for holding or confining the sample or at least a portion thereof. In another aspect, the device may have an inlet port and an outlet port, and the inlet and outlet ports may be both connected with the sample chamber.

In one embodiment, a system and a method for analyzing a sample are disclosed wherein the sample or a portion thereof is loaded onto a device, such as a cartridge. The sample may contain at least one object to be analyzed (also referred to as “target analyte”). The method may include a step of specifically labeling the target analytes with one or more excitable tag, such as a fluorophore. Different kinds of target analytes may be labeled with different excitable tags. In another embodiment, the method may also include a step of allowing the sample to be in contact with the first inner surface, wherein the at least one object is immobilized at the first inner surface. In another embodiment, the at least one object may accumulate or sediment at the first inner surface through the force of gravity. The disclosed method may also include a step of illuminating the objects immobilized at the first substrate using one or more light conditions to generate one or more fluorescence images. The one or more images of the object may be captured and analyzed to identify and/or enumerate objects in a sample.

In another embodiment, the device disclosed herein may be used to receive a sample. In an embodiment, the sample is human blood or its derivative. In one aspect, the sample may be pre-incubated with one or more labeling molecules before being received by the device. The one or more objects in the sample may be bound with one or more labeling molecules when received by the device. The one or more objects in the sample may be labeled by the one or more labeling molecules which may then be identified by the reader instrument. Algorithms may be designed for the reader instrument to enumerate the objects labeled with different labeling molecules.

It is also disclosed here a method for analyzing a sample having one or more analytes or objects, the method may include the following steps: (a) adding the sample or a portion thereof to a device, wherein the sample may contain at least one object. In one aspect, the device may contain a first substrate and a second substrate, wherein the first substrate contains a planar waveguide with a refractive volume. In another aspect, the planar waveguide may have a first outer surface and a first inner surface, and the second substrate may have a second outer surface and a second inner surface. The first inner surface and the second inner surface may be spaced apart from each other and at least partly define a volume (also referred to as “sample chamber) for confining the whole or a portion of the sample. In step (b) the sample may be allowed to be in contact with at least one of the first inner surface and the second inner surface, wherein the at least one object is bound to the first inner surface of the first substrate. In step (c), the at least one object bound to the first inner surface may be detected. In another aspect, the method may further include a step (d) of providing light from a light source to illuminate the refractive volume of the device, wherein the light is coupled to the planar waveguide via the refractive volume.

### BRIEF DESCRIPTION OF THE DRAWINGS

FIGS. 1 and 2 show perspective views of a cell counting system with a cartridge, in accordance with an embodiment.

FIG. 3 is a flow diagram illustrating a method for cell counting, in accordance with an embodiment.

FIGS. 4 and 5 are additional schematic representations of a cell counting system having a cartridge, in accordance with an embodiment.

FIG. 6 is a flow diagram illustrating a sample processing method suitable for use with the method for cell counting, in accordance with an embodiment.

FIG. 7 is a flow diagram illustrating an alternative sample processing method suitable for use with the method for cell counting, in accordance with an embodiment.

FIG. 8 shows a flow chart illustrating the image capture and analysis processes, in accordance with an embodiment.

FIG. 9 shows a flow chart illustrating details of the “Perform autofocus routine” step in FIG. 8, when using a microsphere-based autofocus method, in accordance with an embodiment.

FIG. 10 shows a flow chart illustrating details of the “Perform autofocus routine” step in FIG. 8, when using a fringe-based autofocus method, in accordance with an embodiment.

FIG. 11 shows the details of the “Perform fringe analysis” step of FIG. 10, in accordance with an embodiment.

FIG. 12 shows a cross sectional view of a planar waveguide with a printed spot for use with the autofocus routine, in accordance with an embodiment.

FIG. 13 is a flow chart illustrating details of the “Perform image analysis” step in FIG. 8, in accordance with an embodiment.

FIG. 14 is a flow chart illustrating a process for identifying the locations of the white blood cells in the brightfield and fluorescence images, in accordance with an embodiment.

FIG. 15 shows the results of a comparative study comparing the CD4 cell counts obtained using the present system and industry standard fluorescence activated cell sorting (“FACS”) analysis.

FIG. 16 shows the results of a comparative study comparing the CD4 cell counts obtained using the present system and a dual platform, flow cytometry system, quantifying the comparison by showing the distribution of counts with respect to a Passing Bablok fit.

FIG. 17 shows the results of a reproducibility study, quantifying the variation in CD4 cell counts obtained using the present system on the same blood sample.

FIG. 18 shows the results of a comparison study, comparing the cell counts as measured by flow cytometry and with dried or liquid antibodies in the present cell counting system, in accordance with an embodiment.

FIGS. 19-21 illustrate an alternative way of forming a planar waveguide, such as that shown in FIG. 12.

FIG. 22 illustrates several examples of existing coupling schemes involving multimode waveguides.

FIG. 23 illustrates a generalized configuration descriptive of exemplary embodiments.

FIG. 24 illustrates a cross-sectional view of an exemplary waveguide with an integrated lens.

FIG. 25 provides a detailed cross-sectional view of the waveguide with the integrated lens depicted in FIG. 24.

FIG. 26 is a cavalier projection view illustrating the exemplary waveguide with the integrated lens.

FIG. 27 is a cavalier projection view illustrating an exemplary gasket with multiple channels.

FIG. 28 is a flowchart of an exemplary method for performing sample analysis.

FIGS. 29 and 30 illustrate an exemplary embodiment of a planar low-n core waveguide, with liquid sample containment by two-dimensional surface tension. Within the context of the present disclosure, a planar low-n core waveguide is a planar waveguide in which the core of the waveguide exhibits a lower refractive index than the materials that surround the core.

FIGS. 31 and 32 illustrate an exemplary embodiment of another planar low-n core waveguide, with sample containment by a solid sealing material for four sides and surface tension for two sides.

FIGS. 33-36 are illustrations of light propagating in an embodiment of a planar low-n core waveguide. FIGS. 33 and 34 show collimated light propagating through thick and thin waveguides. FIGS. 35 and 36 show diverging light in thick and thin waveguides. In all figures, partial reflections at various internal interfaces (for example, the substrate-to-interrogation medium interface) have been omitted for clarity.

FIGS. 37-46 show diagrammatic illustrations of variations for light coupling means suitable for use with the planar low-n core waveguide.

FIG. 47 shows an exemplary embodiment, in which the interrogation medium is fully contained by solid material, and the light is coupled into the waveguide itself within the containment region. The dashed line represents an exemplary shape for appropriate light coupling into the waveguide.

FIGS. 48-49 illustrate exemplary embodiments of a planar low-n core waveguide with a liquid interrogation medium, including light coupling means, fluid containment, and fluid inlet and outlet ports. Both substrates may be optically clear for the wavelength range of interest, as shown in FIG. 49. A gasket may be used to contain the liquid in two dimensions, as shown in FIGS. 48-49. Alternatively, the upper component may be shaped to include side walls or stand-offs, which may be directly bonded to the waveguide substrate.

FIGS. 50 and 51 show embodiments similar to that shown in FIG. 49, wherein the upper substrate further includes a reflector.

It is noted that for purpose of clarity, not all elements in the figures may be drawn to scale.

### DETAILED DESCRIPTION

The present disclosure provides a system and method for detecting and characterizing an analyte or an object in a sample. More specifically, this disclosure provides a low-cost, point-of-care system capable of delivering accurate counts of CD4 T helper cells in a small volume of blood sample. In addition to plasma, erythrocytes (i.e., red blood cells or “RBCs”) and thrombocytes (i.e., platelets), blood contains a diversity of leukocytes (i.e., white blood cells or “WBCs”). These include lymphocytes (for example, T, B, and Natural Killer cells), monocytes, and granulocytes (for example, neutrophils, eosinophils, basophils). Leukocytes are characterized by surface proteins called CD markers, and different CD markers are often shared across different leukocyte types. For example, both T helper lymphocytes and monocytes carry the CD4 surface marker. T helper lymphocytes also carry the CD3 marker, while monocytes do not. T helper lymphocytes don't carry the CD14 marker while monocytes do. Differential immunostaining with antibodies against surface CD markers may be used to identify leukocyte subtypes.

The methods described here may be collectively referred to as “static cytometry” using an inventive implementation of planar waveguide fluorescence illumination. The term “cytometry” technically refers to the counting or enumeration of cells, particularly blood cells. The term “cytometry” is used generically in this disclosure to refer to the enumeration of any of a number of analytes, particularly particle analytes, described in more detail below. The term “static” implies that the disclosed system and methods do not require that the target analytes (for example, cells or particles) be moving or flowing at the time of identification and enumeration. This in contrast to “flow cytometry,” a technical method in which target analytes (e.g., cells or particles) are identified and/or enumerated as they move past a detector or sets of detectors. Examples of static cytometry include, for example, hemacytometers such as the Petroff-Hauser counting chamber which is used with a conventional light microscope to enumerate cells in a sample. Cell staining apparatus and fluorescence microscopy instrumentation can be used to perform fluorescence-based static cytometry. The present disclosure provides significantly simplified, robust, and low-cost methods, devices, and instruments for performing static cytometry analysis on a sample.

The methods and systems described here are generally relating to assays that use fluorescence signal to identify and/or enumerate analyte(s) present in a sample. In exemplary applications, target analytes are specifically labeled with fluorophore-conjugated molecules such as an antibody or antibodies (immunostaining). Other molecular recognition elements could also be used, including but not limited to aptamers, affibodies, nucleic acids, molecular recognition elements, or biomimetic constructs. Non-specific fluorophores could also be used, including but not limited to stains such as propidium iodide, membrane specific fluorophores, and fluorescent nuclear stains.

In exemplary embodiments, excitable tags may be used as detection reagents in assay protocols. Exemplary tags may include, but are not limited to, fluorescent organic dyes such as fluorescein, rhodamine, and commercial derivatives such as Alexa dyes (Life Technologies) and DyLight products; fluorescent proteins such as R-phycoerythrin and commercial analogs such as SureLight P3; luminescent lanthanide chelates; luminescent semiconductor nanoparticles (e.g., quantum dots); phosphorescent materials, and microparticles (e.g., latex beads) that incorporate these excitable tags. For the purpose of this disclosure, the term “fluorophore” is used generically to describe all of the excitable tags listed here. The terms “fluorophore-labeled,” “fluor-labeled,” “dye-labeled,” “dye-conjugated,” “tagged,” and “fluorescently tagged” may be used interchangeably in this disclosure.

The embodiments described herein may be applicable to assays beyond fluorescence-based signal transduction. For example, the methods and systems may also be compatible with luminescence, phosphorescence, and light scattering based signal transduction.

In one embodiment, two color fluorescence microscopy based on planar waveguide illumination and differential immunostaining can be used to identify and enumerate analytes in a sample. The present disclosure provides a convenient method and system for performing this analysis. For example differential immunostaining with anti-CD4 and anti-CD14 antibodies may be used to identify CD4 T helper lymphocytes in blood. As another example, differential immunostaining with anti-CD4 and anti-CD3 antibodies may be used to identify CD4 T helper lymphocytes in blood. Optionally, differential immunostaining may be combined with brightfield microscopy and cell morphology analysis to more accurately identify CD4 T helper lymphocytes in blood. As another alternative, differential immunostaining and brightfield microscopy may be used to identify other cell types.

The terms “T cells” and “T lymphocytes” may be used interchangeably in this disclosure. The terms “T helper cells,” “CD4 T helper cells” and “CD4 T cells” may be used interchangeably in this disclosure to refer to those T helper cells that express CD4 on their surface.

For purposes of this disclosure, a cell that binds to a labeling molecule with substantial affinity may be termed “positive” for that particular labeling molecule. Conversely, a cell that does not bind to a labeling molecule with substantial affinity may be termed “negative” for that particular labeling molecule. For instance, a cell that binds an anti-CD4 antibody with a fluorescence tag and shows up as a detectable fluorescence spot when illuminated may be termed “CD4 positive.” Conversely, a cell that does not show up as a detectable fluorescence spot after incubation with an anti-CD4 antibody with a fluorescence tag under the same or similar condition may be termed “CD4 negative.”

Plural or singular forms of a noun may be used interchangeably unless otherwise specified in the disclosure.

In another embodiment, the sample may be incubated with a solution (or mixture) that contains one or more labeling molecules before being loaded onto the cartridge. Examples of the labeling molecules for blood analysis may include but are not limited to an anti-CD4 antibody, an anti-CD14 antibody, an anti-CD3 antibody or an anti-CD8 antibody. In one aspect, different labeling molecules may be incubated with the sample, one by one in a sequential order. In another aspect, different labeling molecules may be incubated with the sample simultaneously. By way of example, an anti-CD4 antibody, an anti-CD14 antibody may be pre-mixed as an antibody cocktail into which the sample or a portion thereof is added. Red blood cell (RBC) lysis buffer may be pre-mixed with the antibody cocktail before the sample is added. In another aspect, the labeling molecule may be one or more antibodies that are tagged or fused with a detectable molecule, such as an excitable tag. Example excitable tags include, but are not limited to fluorescent dyes, fluorescent proteins, lanthanide chelates, quantum dots, and light scattering particles.

In one embodiment, the sample may be a body fluid obtained from a subject. Examples of the samples suitable for the instant system may include but are not limited to whole blood sample, plasma, serum, sputum, bronchoalveolar lavage samples or aspirates, nasopharyngeal swabs, nasal swabs, cerebrospinal fluid (“CSF”), saliva, lymphatic fluid, amniotic fluid, ascites fluid, urine or a combination thereof. In another embodiment the sample may include but is not limited to cultured cells, cell preparations, cell extracts, culture media or combinations thereof. In another embodiment, the sample can be an environmental sample, waste water, industrial waste, or combination thereof.

In an embodiment, samples may contain target analytes. Examples of target analytes include objects with dimensions (e.g., diameters) ranging from 0.1 to 50 micrometers. Examples of target analytes may include mammalian cells. Example of mammalian cells may include white blood cells. Additional examples of target analytes may include viral particles, bacteria, spores, fungi, parasites, liposomes, plant cells, and cellular sub-components.

For certain applications, it is desirable to quantitatively enumerate the number of a specific cell type in a given sample volume. For example, the quantification of CD4 T helper cells in HIV-infected individual typically reports absolute number of cells per microliter of whole blood. The present invention provides means for quantitative cell enumeration.

In one embodiment, controlled volume transfer devices are used to transfer the sample to the inlet port of a cartridge with a fluidic channel wherein the loaded sample may be in contact with one or both of the first inner surface and the second inner surface. Controlled volume transfer devices can be commercially sourced (e.g., Poly-Pipets, Inc, or Safe-Tec Micro-Safe® tubes) or can be custom-designed. In another embodiment controlled volume transfer devices are used to transfer the sample to a sample handling device. In an embodiment for blood cell applications, the sample handling device may be a vial, microtube, or test tube containing a pre-measured, ready-to-use amount of stain and/or lysis buffer used to prepare the sample for assay. For example, the sample handling device may contain a pre-measured, ready-to-use amount of a fluorophore-labeled anti-CD4 and anti-CD 14 antibodies. The sample handling device may also contain a pre-measured, ready-to-use amount of RBC lysis buffer. In another embodiment, the sample handling device may contain lyophilized or otherwise dried assay reagents that are rehydrated with the sample or other liquid at the time of assay.

In another embodiment, prepared sample is transferred from the sample handling device to the inlet port of a cartridge using a transfer device. Exemplary transfer devices include disposable dispensers, disposable transfer pipets, fixed volume pipetters, and adjustable pipetters. In one embodiment, transfer of the prepared sample from the sample handling device to the inlet port of the cartridge is a controlled volume step. In another embodiment, transfer of the prepared sample from the sample handling device to the inlet port of the cartridge is not a controlled volume step.

In another embodiment, prepared sample is transferred from the sample handling device to the inlet port of a cartridge by simple pouring.

In another embodiment, the sample handling device is designed to mate with the inlet port of the cartridge, providing direct transfer of the prepared sample to the cartridge. In one embodiment, the sample handling device may comprise a membrane that can be pierced upon insertion into its mating receptor on the cartridge. For example, the sample handling device may be a vial with a foil adhesive base that holds liquid during sample preparation, but that is pierced when mated to the cartridge, discharging prepared sample.

In another embodiment the cartridge device can be designed such that filling of the fluidic channel is rapid relative to the sedimentation time of objects in the sample. By way of example, fluidic channel geometry can be designed such that a sample comprising immunostained blood fills the channel in a matter of seconds, while sedimentation of target leukocytes occurs over a period of minutes. In this embodiment, sedimentation occurs largely after flow is terminated, and is therefore directional relative to the first inner surface, i.e., sedimentation is perpendicular to the first inner surface when the cartridge is placed on a flat surface. In this embodiment, said directional sedimentation enables on-cartridge volume calibration without the use of any flow metering or fluidic volume control. For example, a known cartridge channel height and a known two-dimensional imaging area (detector field of view) on the first inner surface define a known volume. When sedimentation is perpendicular to the first inner surface and is allowed to go to completion, cells counted on the first inner surface represent all cells in the defined volume and a cell concentration (cells per unit volume) can be calculated.

One advantage of the present disclosure is that a quantitative cell concentration may be generated using only a single controlled volume transfer step. In the example of blood cell enumeration, the only controlled volume step may be the transfer of blood from a capillary finger stick or venipuncture tube. Assuming the known blood sample volume is added a pre-measured, ready-to-use reagent, subsequent transfers and processing do not require controlled volumes. The concentration calculation is based on the known sample dilution and the imaging area approach described above. In another aspect, no on-cartridge metering, pumping, or volume measurements are required for the quantitative assay.

In another embodiment, the first inner surface is modified to create an attachment surface, wherein the modification enhances the affinity between the attachment surface and the analyte or object, such as a mammalian cell. By way of example, the attachment surface may be treated with a polycation such as poly-L-lysine (“PLL”). Alternatively, the attachment surface may comprise other polycations such as poly-D-lysine, specific antibodies, adhesive proteins such as fibronectin, receptor peptides such as RGD, self-assembled monolayers with positively charged, adhesive, or molecular recognition headgroups, polymer films, molecular recognition moieties, or surface activation chemistries such as amino-silanes.

One advantage of the present invention is that devices (cartridges) are processed independently of the reader instrument, enabling batch mode processing of cartridges. This provides a significant throughput advantage over competing technologies in which the instrument is occupied during cartridge processing. In one embodiment, the assay time on the reader instrument is less than 4 minutes, enabling up to 15 samples to be processed per hour on the reader.

In one embodiment, a cartridge rack may be used to simplify cartridge assays. For example, a cartridge rack with defined and labeled cartridge locations can improve organization of multiple cartridges being processed in parallel, reducing errors. In another embodiment, the cartridge rack can have active features further simplifying cartridge processing. For example, insertion of a cartridge into a rack position may actuate a timer and visual cues to the user such as lights and timer status that tell user that an assay step is in process or complete. Audible cues can also be used to alert the user to cartridge status, such as assay completion. In still another embodiment, the cartridge rack may have active feature that physically actuate processes on the cartridge. For example, a wash buffer may be incorporated in a blister pack on the cartridge during manufacturing. In one embodiment, insertion of a cartridge into a rack position may actuate a timer, and after a defined time the rack may further initiate a mechanical actuator (e.g., a ram or lever) that deploys the wash buffer blister pack into the cartridge, completing a timed assay step without any user interaction. An advantage of the present disclosure is that these simple features may be implemented in a highly parallel manner on an “active rack” that independently manages multiple cartridges. Simple logic circuitry and actuator motors may be incorporated at low cost in the rack, leaving the reader instrument available for processing multiple cartridges in series.

In one embodiment, different wavelengths may be used to illuminate the waveguide and the attached analytes or objects. One or more images may be taken of different fields of view on the attachment surface. The reader instrument may contain a translational mechanism to move the device relative to the lens and the image-capturing device so that images of different fields of view may be obtained.

In another embodiment, a brightfield microscopy image is captured for each field of view along with each fluorescence channel. The brightfield image may be used to resolve target objects via morphology analysis.

One of the advantageous features of the disclosed methods and systems is that a relatively small amount of the sample is required for each assay. In the context of blood based assays, it is desirable for the system to be compatible with both venous whole blood and capillary (finger stick) whole blood. In an embodiment, the sample has a specific volume in the range of 1 to 50 microliters, or preferably 1 to 20 microliters, or more preferable 1 to 10 microliters. In one embodiment, 10 microliters of blood sample is required to obtain an accurate count of T helper cells in a sample.

In one embodiment, a sample may be incubated with one or more labeling molecules, such as fluorescence tagged anti-CD4 and anti-CD14 antibodies, and the cells may be enumerated after being loaded onto the cartridge and subjecting to illumination in the reader instrument.

In another embodiment, the reagents disclosed herein may be provided in a kit. In one aspect, the kit may contain, among other things, at least two antibodies. Examples of the antibodies may include anti-CD4 antibody, an anti-CD 14 antibody, anti-CD3 antibody or anti-CD8 antibody. In another aspect, the kit may contain an anti-CD4 antibody and an anti-CD14 antibody. The kit may contain antibodies that have been tagged or fused with an excitable tag, such as a fluorophore. The kit may also contain a tagged secondary antibody that recognizes the primary antibody, such as, for example, the anti-CD4 antibody or the anti-CD14 antibody. The kit may also include RBC lysis buffer at working concentration or at stock concentration. In another aspect, the kit may further contain the device disclosed herein, such as, for example, the cartridge.

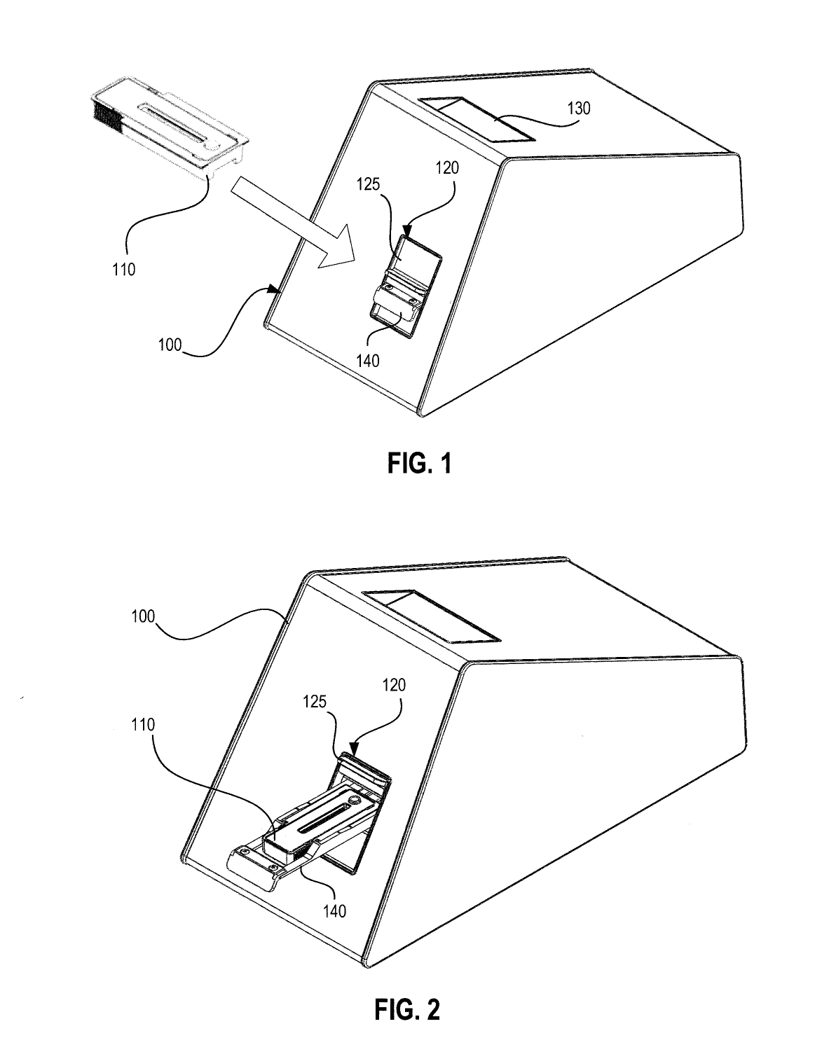

FIGS. 1 and 2 show a cell counting system including a reader instrument 100 with a cartridge 110, in accordance with an embodiment. Cartridge 110 is configured for receiving a sample (not shown), such as blood, serum, environmental sample, as well as associated processing reagents and the like therein. Reader instrument 100 may include a variety of features such as, but not limited to, an opening 120 with a door 125 and a handle 130. Door 125 may be opened in order to insert cartridge 110 into reader instrument 100 for image capture and analysis. Additionally, as shown in FIG. 2, reader instrument 100 may be further equipped with a tray 140 for positioning and insertion of cartridge 110 into reader instrument 100. Reader instrument 100 may have an on-board computer and user interface means such as a touchscreen or keypad (not shown). Alternatively, instrument 100 can operate as a USB peripheral off of a standard personal computer such as a laptop computer.

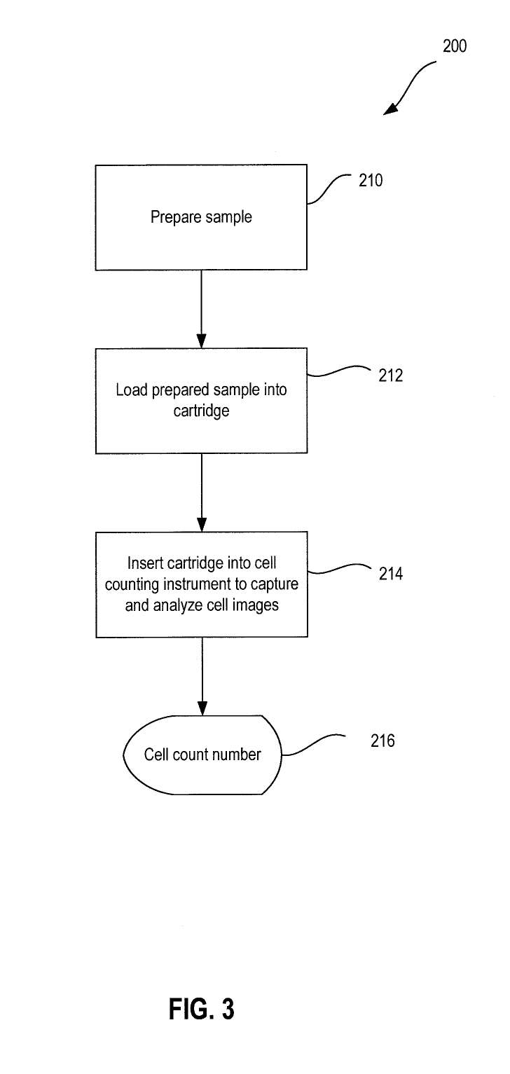

FIG. 3 is a flow diagram illustrating an exemplary method for blood cell counting, in accordance with an embodiment. As shown FIG. 3, a method 200 begins with a step 210 in which the sample to be analyzed is prepared. Preparation step 210 may include, for example, sample collection by venipuncture or other methods, addition of appropriate reagents, and mechanical manipulation of the sample, such as magnetic bead processing, centrifugation or vortexing. Then, in a step 212, the prepared sample is loaded into a cartridge, such as cartridge 110 of FIG. 1. Optionally, additional processing at the cartridge, such as the introduction of reagents, may be performed. For example, raw sample may be added to the cartridge followed by the addition of immunostain reagents and wash buffers. The cartridge is then inserted into a reader instrument, such as reader instrument 100 of FIG. 1, for the capture and analysis of cell images. The reader instrument then produces a cell count number in a step 216. Further details of each of the steps of method 200 are described below.

For certain applications, it may be desirable to quantitatively enumerate the number of a specific cell type in a given sample volume. For example, the quantification of CD4 T helper cells in HIV-infected individual typically reports an absolute number of cells per microliter of whole blood. The present disclosure provides examples for such quantitative cell enumeration.

It would be desirable to have preparation step 210 require minimal user interaction and no external equipment such as vortexers, shakers, or magnets. For instance, in an exemplary method, a whole blood sample may be transferred via a controlled volume transfer device to a sample handling device such as a microtube, vial, or test tube containing pre-measured, ready-to-use assay reagents such stains and buffers. After simple manual mixing via tube inversion or aspiration, the prepared sample is loaded into the cartridge in step 212. An advantage of this exemplary method is that the whole blood sample transfer is the only controlled volume step in the entire process. When a known blood volume is added to a known volume of assay reagent, quantitation is maintained and an absolute cell count may be calculated.

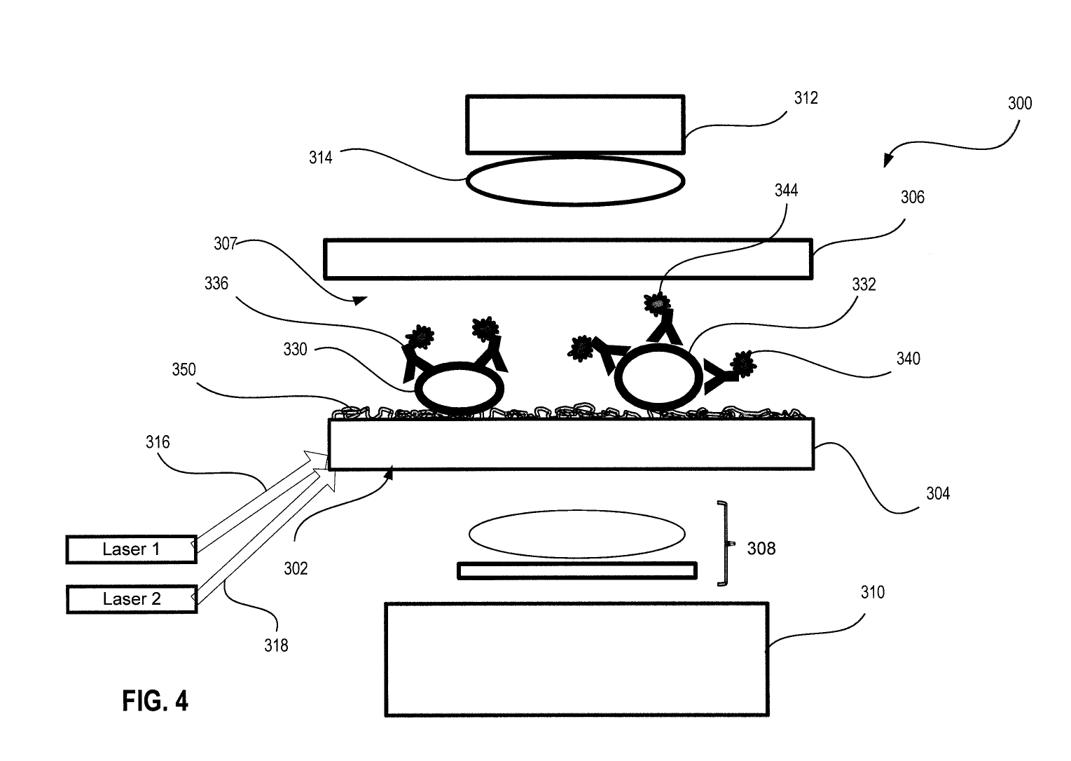

FIG. 4 is a schematic representation of the cell counting system with cartridge for purpose of illustration only. Not all components of the system are shown. A cell counting system 300 is shown with a cartridge 302 inserted therein. Cartridge 302 includes a first substrate 304 and a second substrate 306, which cooperatively define a fluidic channel 307. First substrate 304 may be a planar waveguide, such as described in U.S. patent application Ser. No. 12/617,535, filed 12 Nov. 2009 and entitled “Waveguide with Integrated Lens.” First and second substrates 304 and 306 are configured for containing a fluidic sample therebetween.

Cell counting system 300, as shown in FIG. 3A, may include imaging optics 308, such as but not limited to filters, refractive elements, reflective elements, and holographic elements, for imaging fluorescence or other light signal from cartridge 302 at an image sensor 310. Image sensor 310 may be, for instance, a charge-coupled device (“CCD”) or a complementary metal-oxide-semiconductor (“CMOS”) sensor. Light for brightfield microscopy of objects in cartridge 302 may be generated using light provided by a light emitting diode (“LED”) 312, which is directed to cartridge 302 by optics 314. Other light sources, such as Lasers 1 and 2 providing laser light 316 and 318 (indicated by arrows) can be used to induce fluorescence from objects that are intrinsically fluorescent or that have been labeled with fluorophores. Images captured under different illumination conditions may be used to analyze to provide a cell count number, as will be described in further detail below. Computers may be integrated into the detection system instrument (e.g., on-board computer). Alternatively, the computer may be an external device.

A sample containing various cells 330 and 332 is stained with an antibody 336. Note that more than one type of antibodies may be used. Excitable tags 340 and 344 having different colors may be used to label the antibody. Antibody 336 only binds to cell 330, but not to cell 332. Also, antibody 336 is only labeled with tag 340, but not by tag 344. An inner surface of first substrate 336 has been treated to form an attachment surface 350, which is capable of binding cells 330 and 332.

The sample may be, for instance, a body fluid obtained from a subject. Examples of samples suitable for the instant system may include, but are not limited to, whole blood sample, plasma, serum, sputum, bronchoalveolar lavage samples or aspirates, nasopharyngeal swabs, nasal swabs, cerebrospinal fluid (“CSF”), saliva, lymphatic fluid, amniotic fluid, ascites fluid, urine and a combination thereof. Alternatively, the sample may be cultured cells, cell preparations, cell extracts, culture media and combinations thereof. Furthermore, the sample may be an environmental sample, waste water, industrial waste, food, bacterial sample or combination thereof.

In an embodiment, samples may contain target analytes. Examples of target analytes include but are not limited to objects with dimensions (e.g., diameters) ranging from 0.1 to 50 micrometers. Examples of target analytes may include mammalian cells. Example mammalian cells may include white blood cells. Additional examples of target analytes may include viral particles, bacteria, spores, fungi, parasites, liposomes, plant cells, and cellular sub-components.

The cartridge may be designed such that filling of the fluidic channel is rapid relative to the sedimentation time of analytes in the sample. By way of example, the fluidic channel geometry may be designed such that a sample, including immunostained blood, fills the channel in a matter of seconds, while sedimentation of target leukocytes occurs over a period of minutes. In this example, sedimentation occurs largely after sample flow through the fluidic channel is terminated and, consequently, the sedimentation is substantially perpendicular to the first inner surface of the first substrate when the cartridge is placed on a flat surface. In other words, directional sedimentation enables on-cartridge volume calibration without the use of any flow metering or fluidic volume control.

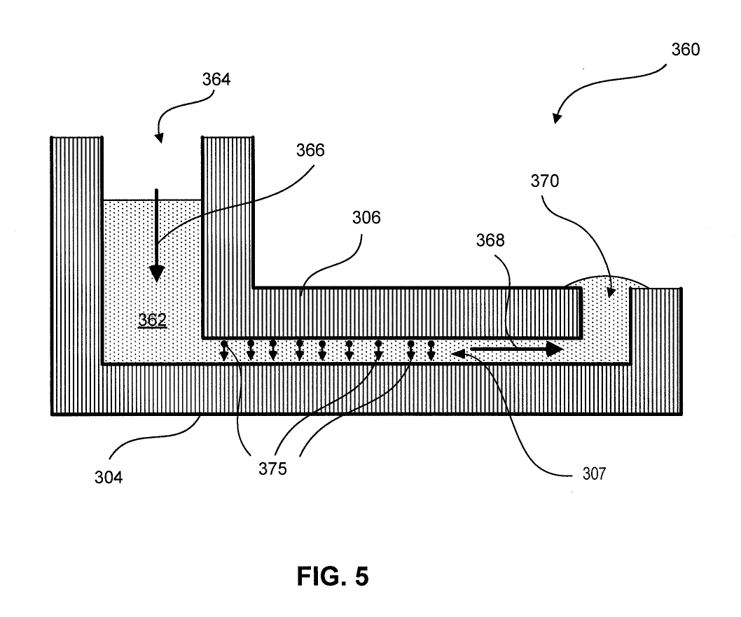

FIG. 5 is a schematic representation of a portion 360 of an exemplary cartridge, such as that partially shown in FIG. 4, in accordance with an embodiment. As shown in FIG. 5, a sample fluid 362 is introduced into fluidic channel 307 from an inlet port 364. Sample fluid 362 flows through fluidic channel 307 as indicated by arrows 366 and 368, then any overflow exits from an outlet port 370. The geometry of fluidic channel 307 may be designed such that sample fluid 362 fills fluidic channel 307 rapidly relative to the gravitational sedimentation time of analytes 375 (indicated by dots with downward arrows) in sample fluid 362. Filling rate for a fluidic channel may be engineered, for example, by configuring the geometry of inlet port 364 to establish a head pressure, by controlling the cross-sectional geometry of fluidic channel 307, and by controlling surface energy (e.g., wettability) of the channel walls. By way of example, the geometry of fluidic channel 307 may be designed such that a sample fluid, including immunostained blood, fills fluidic channel 307 in a matter of seconds, while sedimentation of target leukocytes (i.e., analytes 375) occurs over a period of minutes. Methods for controlling or engineering surface energy (e.g. wettability) of the channel walls may include plasma treatments (e.g., oxygen plasma) or deposition of thin film chemistries.

When the filling rate of fluidic channel is rapid relative to sedimentation, sedimentation occurs largely after flow is terminated. Consequently, the sedimentation is therefore directional relative to the first inner surface, i.e., sedimentation is perpendicular to the first inner surface when the cartridge is placed on a flat surface. Directional sedimentation enables on-cartridge volume calibration without the use of any flow metering devices or fluidic volume control.

In an embodiment, a known fluidic channel height and calibrated field of view of image sensor may cooperatively define a known three dimensional sample volume so as to enable quantitation of cell count within the sample volume. For example, once an immunostained blood sample is introduced to the cartridge, cells within the sample gravitationally settle (i.e., sediment) to the inner surface of the first substrate where they are imaged. Assuming all cells in a given field of view are the result of directional settling from the liquid volume directly above the field of view, the cell count in the two dimensional image may be used to calculate the concentration of cells in the liquid volume as defined by the field of view and the channel height. Note that some lateral motion of fluid during sedimentation is acceptable, as long as cells enter and exit the sampling volume at the same rate. In a particular example, the height of fluidic channel 307 is 0.14 mm and the field of view of the imaging system is 2 mm×2 mm, yielding a “sampled” volume of 0.56 microliters. Cell counts can therefore be expressed as cells per unit volume. In the blood cell counting example described above, a volumetric transfer of blood to a pre-measured stain results in a known dilution factor. Upon transfer of the prepared sample to the cartridge, image-based enumeration generates a count per unit volume. Back calculation with the known dilution factor allows cell concentration in the original blood sample to be established.

The first inner surface of fluidic channel 307 may be modified to create an attachment surface, wherein the modification enhances the affinity between the attachment surface and the analyte or object, such as a mammalian cell. By way of example, the attachment surface may be treated with a polycation such as poly-L-lysine (“PLL”). Alternatively, the attachment surface may comprise other polycations such as poly-D-lysine, specific antibodies, adhesive proteins such as fibronectin, receptor peptides such as RGD, self-assembled monolayers with positively charged, adhesive, or molecular recognition headgroups, polymer films, molecular recognition moieties, or surface activation chemistries such as amino-silanes.

In one approach, the sample may be incubated with a solution (or mixture) that contains one or more labeling molecules before being loaded onto the cartridge. Examples of the labeling molecules for blood analysis may include, but are not limited to, an anti-CD4 antibody, an anti-CD14 antibody, an anti-CD3 antibody and an anti-CD8 antibody. In one aspect, different labeling molecules may be incubated with the sample, one by one in a sequential order. In another aspect, different labeling molecules may be incubated with the sample simultaneously. By way of example, an anti-CD4 antibody, an anti-CD 14 antibody may be pre-mixed as an antibody cocktail into which the sample or a portion thereof is added. RBC lysis buffer may be pre-mixed with the antibody cocktail before the sample is added. RBC lysis buffer may be, for example, ammonium chloride, hemolysin proteins and recombinants thereof, surfactants and salts. In another aspect, the labeling molecule may be one or more antibodies that are tagged or fused with a detectable molecule, such as an excitable tag. Example excitable tags include, but are not limited to fluorescent dyes, fluorescent proteins, lanthanide chelates, quantum dots, and light scattering particles.

The reagents disclosed herein may be provided in a kit, which may contain, among other things, at least two antibodies. Examples of the antibodies may include anti-CD4 antibody, an anti-CD14 antibody, anti-CD3 antibody or anti-CD8 antibody. In another aspect, the kit may contain an anti-CD4 antibody and an anti-CD14 antibody. The kit may contain antibodies that have been tagged or fused with an excitable tag, such as a fluorophore. The kit may also contain a tagged secondary antibody that recognizes the primary antibody, such as, for example, the anti-CD4 antibody or the anti-CD14 antibody. The kit may also include RBC lysis buffer at working concentration or at stock concentration. In another aspect, the kit may further contain the device disclosed herein, such as, for example, the cartridge.

One of the advantageous features of the disclosed device is that a relatively small amount of the sample is required for each assay. For instance, only about 10 microliters of blood sample is required to obtain an accurate count of T helper cells in a sample.

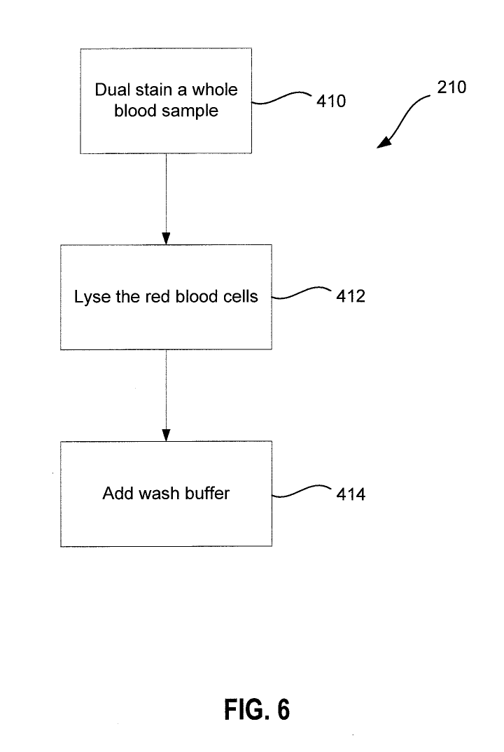

In an example, a two-color laser fluorescence imaging system is used to determine the T-helper cell concentration in a human whole blood sample. FIG. 6 illustrates details of steps involved in “prepare sample” step 210 of FIG. 3. As shown in FIG. 6, target cells in a whole blood sample are stained with two different antibodies conjugated with different fluorescent dyes in a step 410. The red blood cells in the sample are lysed in a step 412. After an addition of a wash buffer in a step 414 in order to remove unattached cells and antibodies, the cartridge is ready for insertion into the reader instrument for image capture and analysis in step 212 of FIG. 3.

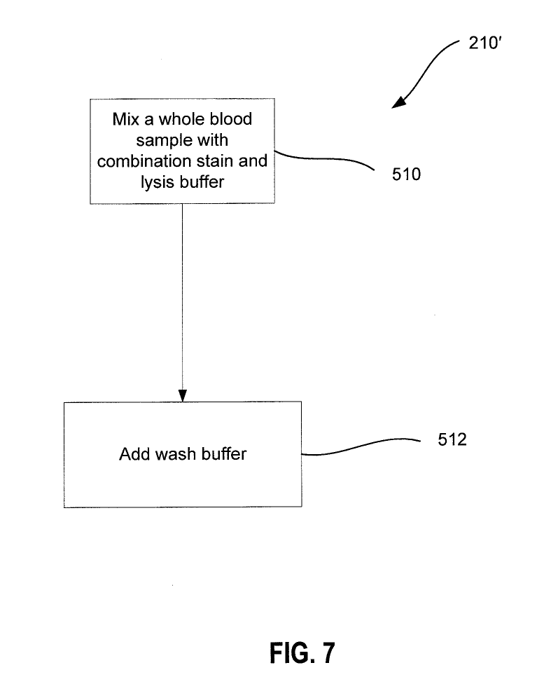

A simpler alternative to “prepare sample” step 210 of FIG. 3 is shown in FIG. 7. FIG. 7 shows details of an alternative “prepare sample” step 210′, which involves only two fluid additions to the cartridge. As shown in FIG. 7, a whole blood sample is mixed with a combination stain and lysis buffer in a step 510. Then, after a wash buffer is added in a step 512, the cartridge may be inserted into the reader instrument in step 212 of FIG. 3. Further details of the alternatives of the prepare sample step are described in the Examples discussed below.

One advantage of the present invention is that devices (cartridges) are processed independently of the reader instrument, enabling batch mode processing of cartridges. This provides a significant throughput advantage over competing technologies in which the instrument is occupied during cartridge processing.

The reader instrument may use different wavelengths to illuminate the waveguide and the attached analytes or objects so as to enable differential staining analysis. One or more images may be taken of different fields of view on the attachment surface. The reader instrument may contain a translational mechanism to move the device relative to the lens and the image-capturing device so that images of different fields of view may be obtained. Furthermore, a brightfield image may be captured for each field of view along with each fluorescence channel. The brightfield image may be used to resolve target objects via morphology analysis.

The cell counting system may be used to establish absolute CD4 T helper cell count (in units of cells per microliter) in a whole blood sample. For instance, anti-CD4 and anti-CD14 antibodies labeled with two different fluorescent labels may be used to differentially stain a blood sample. Cells that are stained with both the anti-CD4 and anti-CD14 stains are monocytes. Cells that are stained with anti-CD4 are either CD4 T helper cells or monocytes. By subtracting the monocyte count from the overall CD4 count, a CD4 T helper cell count can be generated. Alternatively, other differential staining schemes such as an anti-CD4 and anti-CD3 approach can be used to generate a CD4 T helper cell count.

Additionally, brightfield microscopy may be used to improve accuracy of cell counts generated by the system. In any blood preparation, artifacts such as cellular debris, aggregated dye, insoluble particles, lint, etc. can compromise counts based only on fluorescence images. Brightfield microscopy may be used to discriminate objects based on morphology features such as diameter, area, shape, and optical density. For example, fluorescent particle aggregates comprising cellular debris and stain may appear as a bright object in a fluorescence image, but may have minimal or no structure in brightfield and will therefore not meet criteria established for identifying a cell.

In an example, following the “prepare sample” step, the cartridge may then be imaged in a reader instrument containing two or more different illumination sources such as, for example, a combination of: 1) a red laser (635 nm); 2) a green laser (532 nm); and 3) a broad spectrum light emitting diode (“LED”). Since the cells of interest are captured at attachment surface 350 of first substrate 304, as shown in FIG. 4, it is then necessary to configure imaging optics 308 and image sensor 310 to be focused at attachment surface 350. Imaging optics 308, which serve to image the different fields of view at the sensor, may include, for example, one or more refractive elements, such as lenses and objectives, and phase-modifying elements, filters, and reflective elements. Once focus is achieved, three images corresponding to the three different illumination sources are captured, and the captured images are processed to extract the cell count number in step 216 of FIG. 3.

The reader instrument, in the present example, is designed to generate three spatially-registered digital images for a given field of view. The term “spatially-registered” means that each object in a given image has approximately the same physical location in the other registered image. Deviations from perfect image registration can be corrected using digital image processing algorithms familiar to those normally skilled in the art. For each imaging mode, an identified object is given a spatial location index in software. Each unique object, therefore, has a status associated with each imaging mode. In the brightfield microscopy mode, an object has a physical location and morphology features such as diameter, area, or shape. Digital image processing of the brightfield image is described in more detail below. In each of the fluorescence modes, a given object at a particular physical location may or may not appear in the image, depending on the fluorescence labeling of the object. In software, a table of characteristics for each object in a given field of view is generated. This process is described in more detail below and in the Examples.

An advantage of the present invention is that the three spatially-registered images for each field of view may be generated without mechanical motion of parts in the reader instrument. For example, imaging optics 308 may include a dual bandpass fluorescence emission filter that passes fluorescence from the two fluorescent tags used but blocks both laser excitation wavelengths. The wavelength of the brightfield LED may be selected such that the LED emission passes through the dual bandpass filter so as to additionally enable brightfield image generation. With this optical system configuration, the three images are generated simply by toggling the three light sources (LED, Laser 1, Laser 2) on and off while capturing the resulting images at the same image sensor. In this way, by eliminating the need for moving optical components, such as movable mirrors and filter wheels, the present configuration may be both fast and robust.

While spatially-registered images for each field of view may be generated without moving parts, mechanical translation inside the reader instrument may be implemented to move the cartridge through multiple fields of view to increase the total cell counts and statistical accuracy. Typically, 5 to 20 fields of view are analyzed, and the number of fields of view to capture and analyze may be adjusted according to specific needs. An exemplary instrument utilizes nine imaging fields of view to develop a composite sample volume that delivers absolute counts with statistical significance at the low end of the clinically valuable range (e.g., at 200 cells/microliters, statistical error is +/−20 cells, or 10%). The combination of instrument optical design and cartridge flatness may enable all fields of view to be in focus and imaged after a single initial autofocus operation, improving read speed.

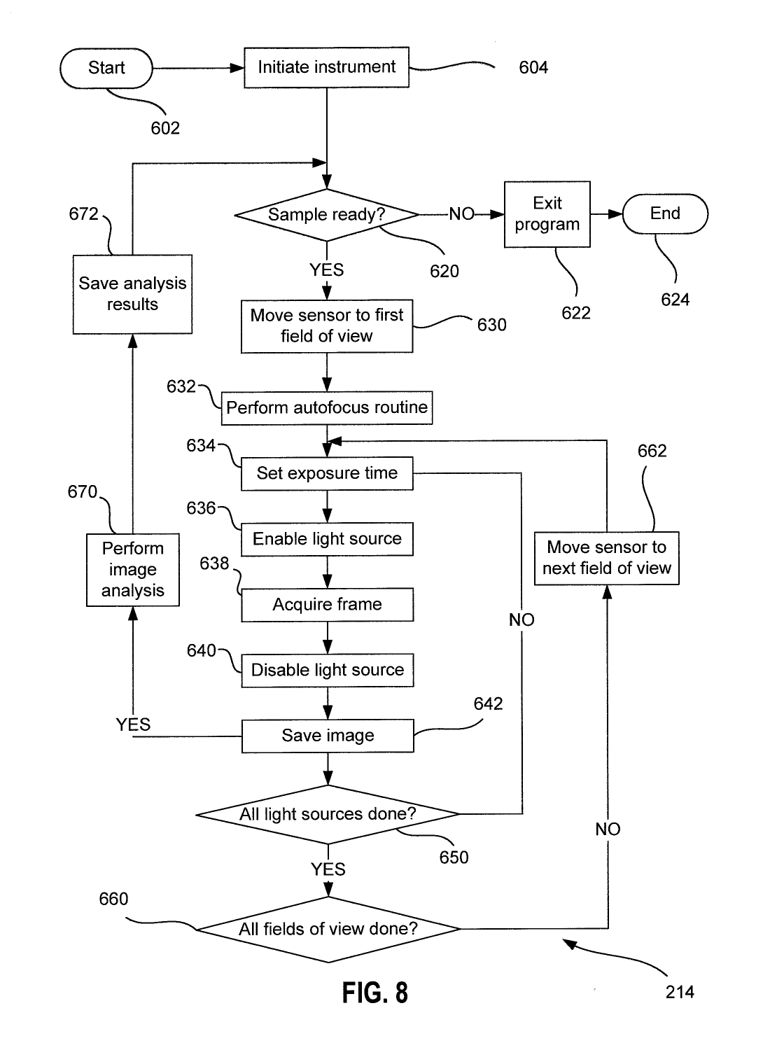

FIG. 8 outlines an exemplary process for image capture and analyses suitable for use in step 214 of FIG. 3, in accordance with an embodiment. The exemplary process is initiated at a start step 602, and various components of the reader instrument are initiated in a step 604. The “initiate instrument” step may include, for instance, connecting a sensor (i.e., any suitable image capturing device) and stepper motors (or other appropriate motion control mechanism), and calibration thereof. Any sensor calibration data, as stored in an external file, may also be read in step 604. After the instrument initialization, a decision 620 is made to determine whether a sample, loaded in a cartridge, is ready for measurement within the imaging system. If the answer to decision 620 is “NO” the sample is not ready, then the process proceeds to exit the program in a step 622, and the process is ended in an end step 624.

Continuing to refer to FIG. 8, if the answer to decision 620 is “YES” the sample is ready, and then the process proceeds to move the sensor to a first field of view of interest in the cartridge in a step 630. An autofocus routine is performed in a step 632, the exposure time of the sensor is set in a step 634, one of the three light sources is enabled in a step 636, and then an image frame of the first field of view of interest is acquired in a step 638. The light source enabled in step 636 is then disabled in a step 640, and the image frame so captured is saved in a step 642. Then in a decision 650, it is determined whether or not all light sources have been used in the image capture.

If the answer to decision 650 is “NO” not all light sources have been used, then the process returns to step 634. If the answer to decision 650 is “YES” all light sources have been used, then the process proceeds to a decision 660 to determine whether all fields of view have been captured. If the answer to decision 660 is “NO” not all fields of view have been captured, then the process proceeds to a step 662 to move the sensor to the next field of view, and steps 632-650 are repeated. If the answer to decision 660 is “YES” all fields of view have been captured, then analysis of the captured images is performed in a step 670, and the analysis results are saved in a step 672. The process then returns to before decision 620. If another sample has been loaded into the imaging system, then the answer to decision 620 is again “YES”, and the process again proceeds from step 630. If no other sample has been loaded into the imaging system, then decision 620 is “NO” and the process is ended.

We have demonstrated two different autofocus methods. One method, referred to as microsphere-based autofocus, uses fluorescent microspheres which settle to the bottom surface just like the cells of interest. The other, referred to as fringe-based autofocus, utilizes a fluorescent coating on the bottom surface to generate fringes that may be used in the autofocusing process. Both autofocus methods are described in detail below. Other autofocus methods may be utilized. For example, fiducial features molded, embossed, or printed into the cartridge may be detected and used for autofocusing purposes. Optical methods, such as those used in digital photography, may also be incorporated into the cell counting system, in an embodiment.

Microsphere-based Autofocus and Associated Object Identification:

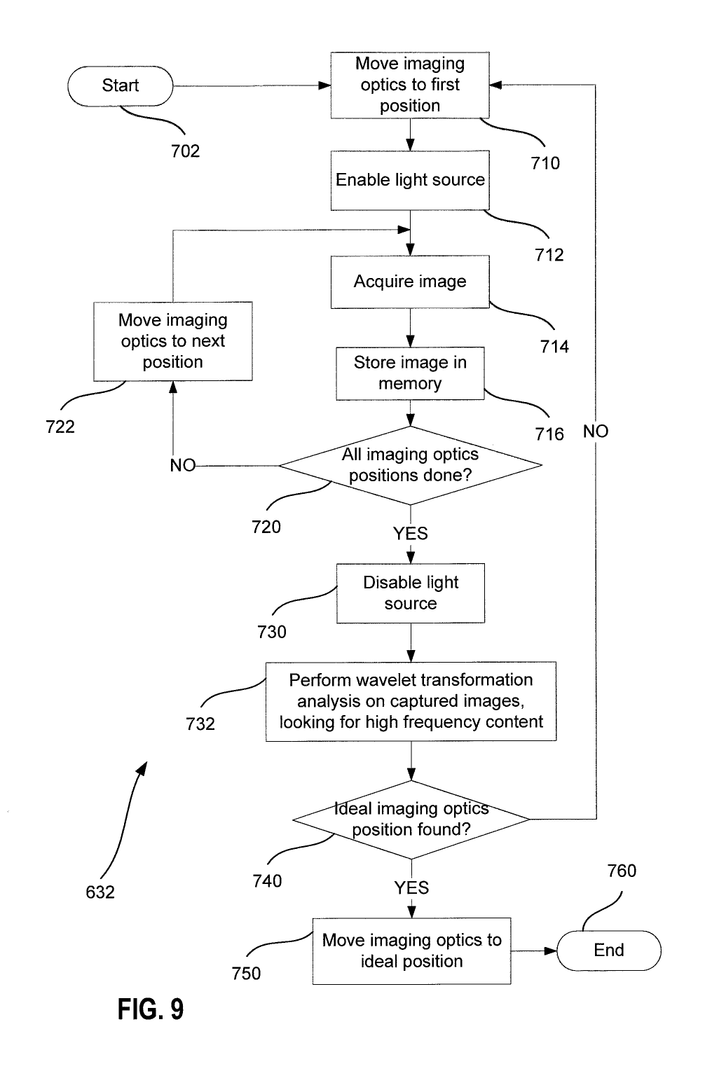

In this method, the cartridge contains both cells and fluorescent microspheres. The microspheres have been chosen such that they settle to the bottom of the fluidic channel, just like the cells, and fluoresce significantly brighter than the cells in the red channel (we have used 6 μm “Flash Red” polystyrene microspheres from Bangs Laboratories). The imaging system can therefore be tuned to see only the microspheres in the red channel, which allows for autofocus based on the appearance of the microspheres using the autofocus routine illustrated in FIG. 9.

FIG. 9 illustrates an example of a process suitable for use as the autofocus routine in step 632 of FIG. 8. The exemplary process is initiated at a start step 702, and imaging optics are moved to a first focus position in a step 710. The specific light source used for autofocusing is enabled in a step 712. An image of the field of view at the first focus position is acquired in a step 714, and the image is stored in memory in a step 716. In a decision 720, a determination of whether or not an image has been captured at all relevant positions of the imaging optics.

If the answer to decision 720 is “NO” images have not been acquired at all relevant imaging optics positions, then the process proceeds to move the imaging optics to the next position, in a step 722, and steps 714-716 and decision 720 are repeated. As a result, the imaging optics are moved through a pre-determined series of focus positions. Once all focus positions have been sampled, the answer to decision 720 is “YES” and then the process proceeds to a step 730, in which the light source is disabled. Then a wavelet transformation analysis is performed on the captured images in order to look for high frequency spatial content in the images, in a step 732. The optimal focus position is related to the imaging optics position in which the high-frequency spatial content of the acquired image is maximized.

Based on the result of the wavelet transformation analysis in step 732, a decision 740 is made to determine whether an ideal position for the imaging optics has been found. If the answer to decision 740 is “NO” an ideal position has not been found, then the process returns to step 710, and steps 710-722 are repeated. If the answer to decision 740 is “YES” an ideal position has been found, then the imaging optics are moved to the ideal position in a step 750 and the process is ended in an end step 760.

After completion of the autofocus procedure, the imaging system is optimized for cell imaging and three images are recorded corresponding to three different light sources: 1) red fluorescence; 2) green fluorescence; and 3) brightfield (see FIG. 8). Events are identified in each of the three registered images and can be correlated for differentiating cell types in the sample. It is beneficial to minimize the number of microspheres in the fields of view used for cell imaging. There is a trade-off between cell counting simplicity and autofocus reliability. When the number of microspheres in a field of view drops below a certain level, the autofocus routine can fail. If the bottom surface is sufficiently flat, then it is possible to move between some fields of view without needing to refocus. The microsphere concentration can then be lowered to the point where it is sufficient for autofocus only in some of the imaged fields of view. If the routine fails in the field of view under consideration, move to neighboring fields of view and keep on in that manner until a field of view is found with enough microspheres. When this is found, perform the autofocus, move back to the field of view initially under consideration, and perform the cell imaging.

Fringe-based Autofocus and Associated Object Identification to Obtain:

In this method, nothing is added to the sample to aid autofocus. Instead the method utilizes a fluorescent coating on the attachment surface, i.e., the surface at which the cells of interest are captured. We have demonstrated that non-specific binding of red fluorophores provides sufficient signal. We have also demonstrated that it is possible to deposit fluorescent material in a thin surface coating onto the substrate and achieve a sufficient signal that way. The coherent nature of the laser beam produces clear fringes in the thin surface coating. The fringe contrast is rapidly reduced when deviating from optimal focus, providing a means for autofocusing on the bottom surface. A series of images are recorded at different focus settings of the imaging system, which may be implemented, for example, by varying the objective-to-cartridge distance.

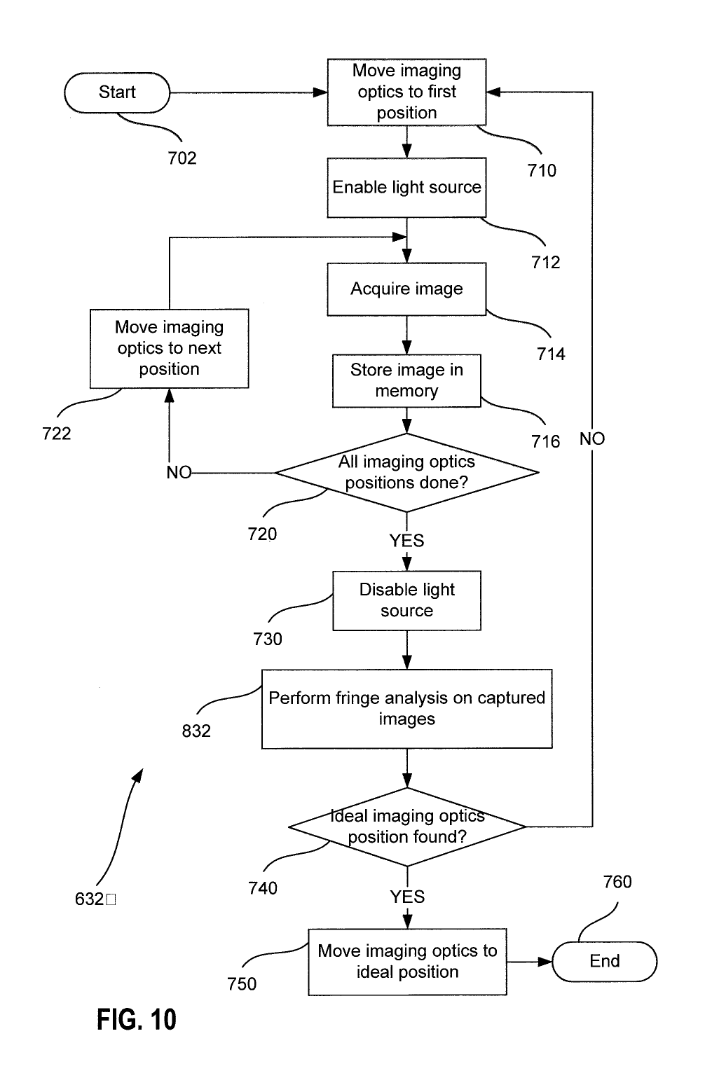

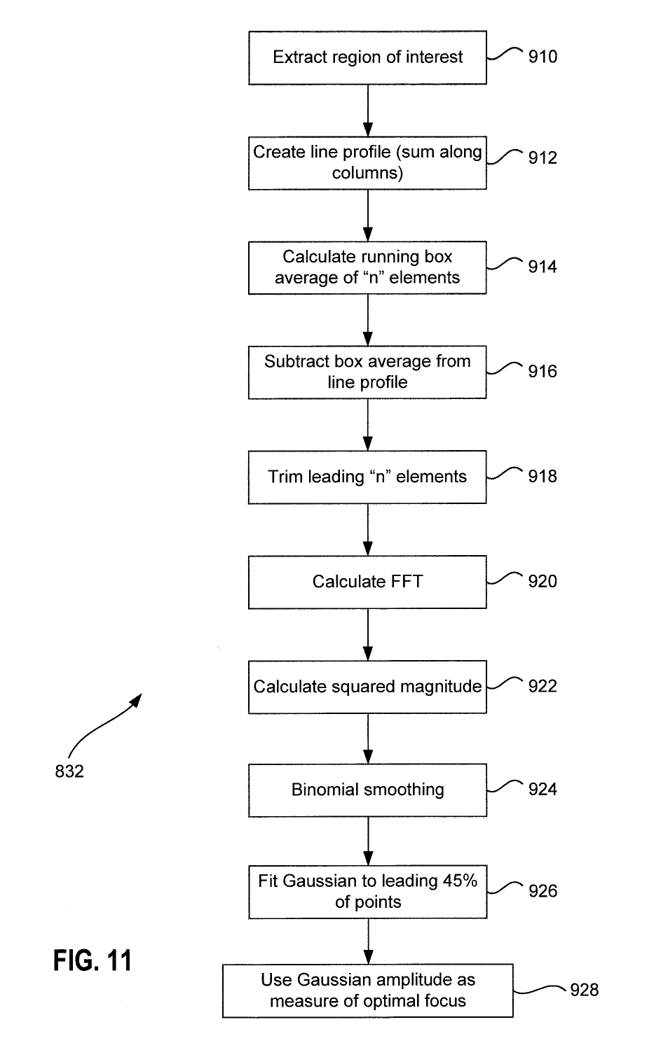

Alternatively, the fringe autofocus may be based on a one or more deposited “focus spots” on the attachment surface. The advantage of the focus spot deposition approach is that fringe signal is bright and reproducible, improving the robustness of the autofocus routine. The images are analyzed according to the procedure outlined in FIGS. 10 and 11.

FIG. 10 illustrates an example of a process suitable for use as the autofocus routine in step 632 of FIG. 8 for fringe-based autofocus. A process 632′ is identical to process 632 detailed in FIG. 9, except the wavelet transformation analysis step has been replaced with a fringe analysis step 832.

FIG. 11 illustrates further details of fringe analysis step 832 of FIG. 10. In a step 910, a region of interest is extracted from the images captured in the autofocus process. Then, a line profile (e.g., sum along columns of the image pixels) is created, in a step 912. A running box average of a number of elements “n” (which is sufficiently greater than the number of pixels between neighboring fringe peaks) is calculated in a step 914. The box average is subtracted from the line profile in a step 916 to remove the slow variation in the line profile created in step 912. The leading “n” elements are trimmed in a step 918. A Fast Fourier Transform (“FFT”) is performed in a step 920 on the truncated line profile. The squared magnitude of the FFT result is calculated in a step 922, then a binomial smoothing is applied in a step 924. A Gaussian fit is performed on the leading 45% of the resulting smoothed FFT in a step 926, and the Gaussian amplitude is used as a measure of optimal focus in a step 928. In other words, the location of the imaging optics where the Gaussian amplitude peak is maximized is considered the ideal position for the imaging optics.

The analysis stages of the two different autofocus routines may occasionally fail to find an ideal position for the imaging optics. For example, the Gaussian fit step 926 may be unable to fit a Gaussian profile to the data points. As another example, more than one peak may be found after the Gaussian fit, thereby leading to confusion regarding which peak is the appropriate indicator of the ideal position for the imaging optics. If such a failure occurs, then the autofocus process is repeated, as shown in FIG. 9.

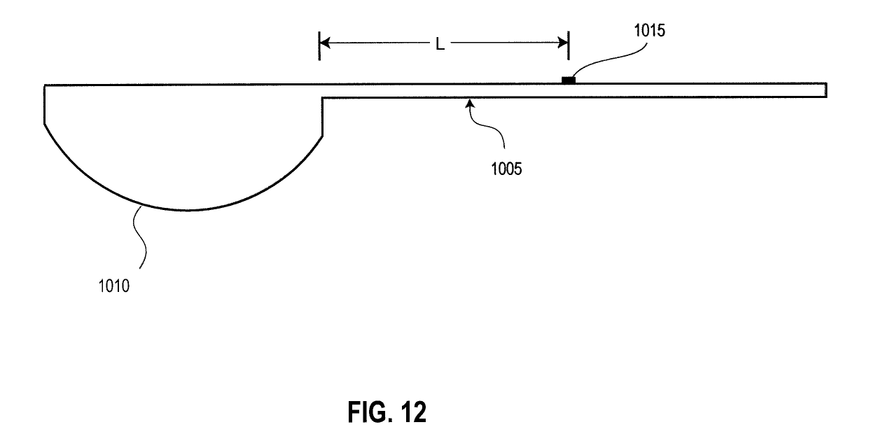

FIG. 12 shows a cross sectional view of a planar waveguide with a printed spot for use with the autofocus routine, in accordance with an embodiment. An exemplary material for the printed autofocus spot is a fluorescent dye, such as DyLight649 (Thermo). A planar waveguide 1005, as shown in FIG. 12, may include an integrated lens 1010. A spot 1015 may be deposited by, for example, using a non-contact printer such as those produced by BioDot, Inc. or similar equipment, at a specific distance L from integrated lens 1010. In one example, L=31.75 mm, which corresponds to a center of the fifth of nine fields of view across the cell attachment surface. When the cell attachment surface is a polyamine polymer such as poly-L-lysine, it may be desirable to print an amine-reactive reagent as the autofocus spot. An exemplary material is a succinimidyl ester modified organic dye, such as NHS-DyLight649.

Exemplary Image Analysis Routine for Blood Cell Counting

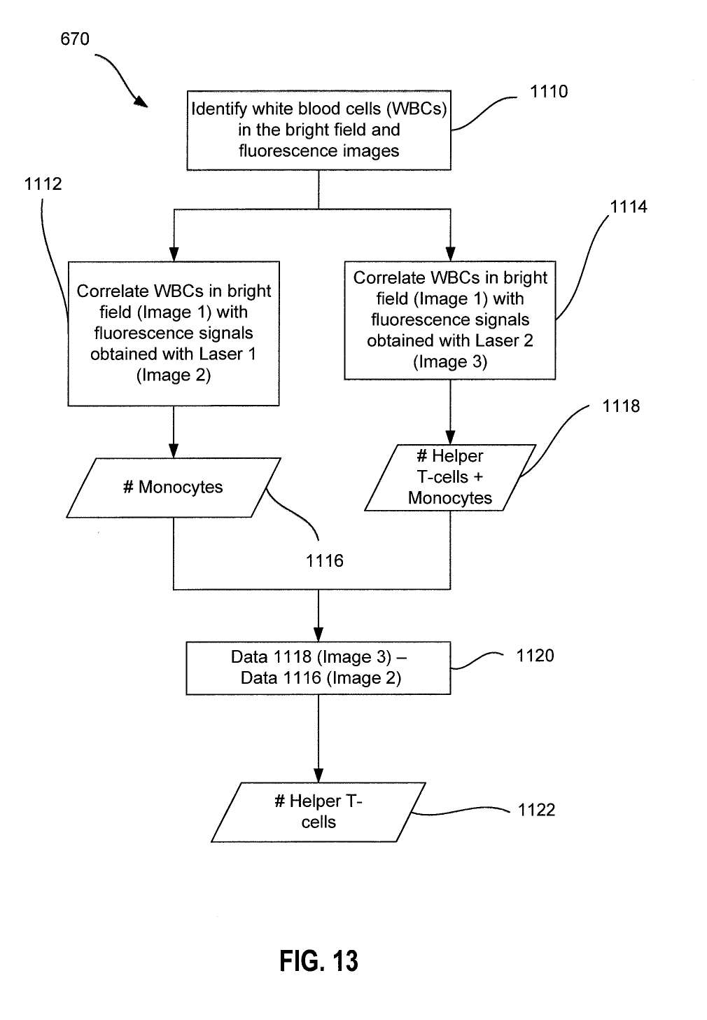

FIG. 13 is a flow chart illustrating details of “Perform image analysis” step 670 in FIG. 8, in accordance with an embodiment. The exemplary process, as shown in FIG. 13, assumes three images, under different illumination conditions, have been captured and stored for each field of view. In the illustrated example: Image 1) brightfield image using illumination with LED 312 and optics 314; Image 2) fluorescence image obtained using illumination with Laser 1; and Image 3) fluorescence image obtained using illumination with Laser 2. In a particular example, LED 312 may be a red LED, which has been selected to generate light at a wavelength transmissible through an emission filter in the band designed for the excitation emission from AlexaFluor 649. Laser 1 may be a diode laser emitting at a red wavelength that is compatible with the excitation of AlexaFluor 649, and Laser 2 may be a diode laser emitting at a green wavelength that is compatible with the excitation of R-phycoerythrin. The image analysis routine in this example may be suitable for enumeration of CD4 T helper cells in a human blood sample.

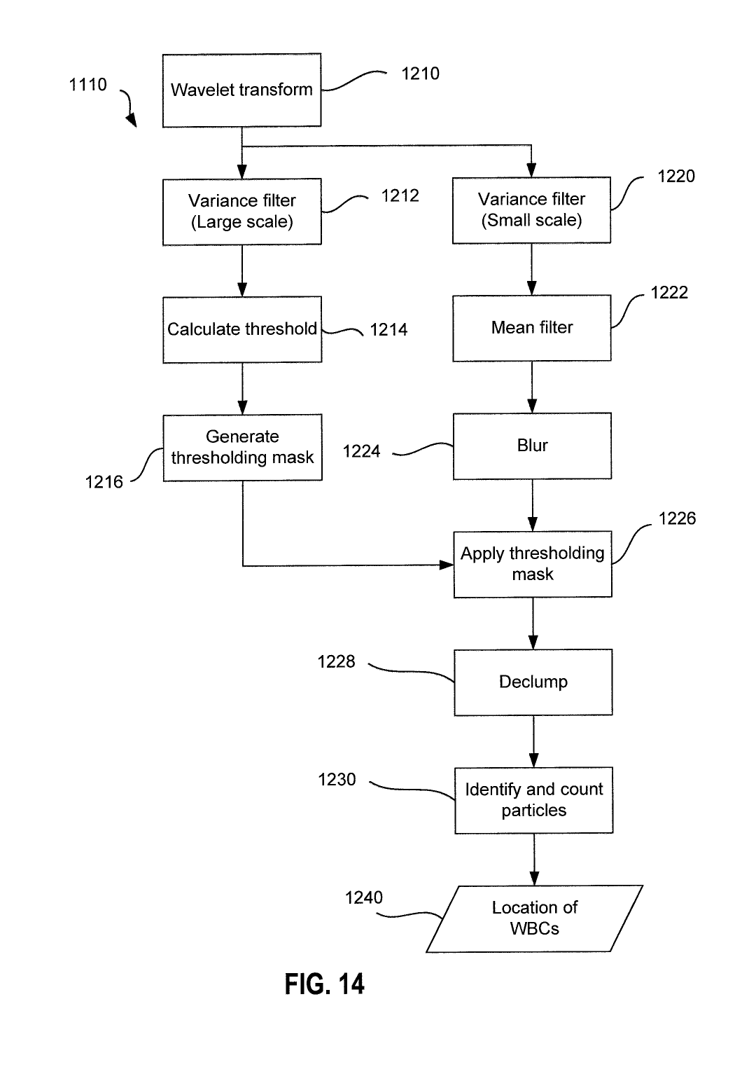

Continuing to refer to FIG. 13, the exemplary image analysis process begins with a step 1110 to identify white blood cells (“WBCs”) in the brightfield and fluorescence images. Step 1110 essentially imposes an object recognition algorithm on the captured images to identify objects that fall within a known range of parameters, as will be described in further detail in FIG. 14.

As shown in FIG. 14, step 1110 includes a brightfield image object recognition routine. A wavelet transform step 1210 provides initial noise reduction pre-processing. A variance filter is applied over a large area of this pre-processed image in a step 1212, and a threshold is established in a step 1214 in order to generate a thresholding mask in a step 1216. Simultaneously, the pre-processed image from step 1210 is processed with a small area variance filter in a step 1220, followed by a mean filter in a step 1222 and a blurring routine in a step 1224. At this point, the threshold mask from the large area variance filter is applied to the resulting image from step 1224 in a step 1226. The processed image is then subjected to a de-clumping routing in a step 1228. Morphological filters, such as size and circularity filters, may then be applied in order to identify and count the particles of interest in a step 1230. The morphological filter eliminates debris, sub-cellular components and any red blood cells that survived the lysis process. The result is data 1240, including a table of object locations all of which are assumed to be white blood cells (“WBC”s).

Once the WBC locations have been identified in the brightfield image, registered fluorescence images are generated with the two laser channels. A filtering algorithm, similar to that shown in FIG. 14, is also applied to the two fluorescence images (i.e., Images 2 and 3) to generate locations of the fluorescing objects in those images. Referring back to FIG. 13, images 2 and 3 are correlated with those in the brightfield image in steps 1112 and 1114, respectively. In an example, fluorescence Image 2 shows cells stained with the anti-CD14 antibody, representing all monocytes, and Image 3 represents all cells stained with the anti-CD4 antibody, thus representing both monocytes and CD4 T Helper cells. In FIG. 13, step 1112 results in data 1116, representing the physical location and number of monocytes (e.g., CD14+ cells) in the field of view. Step 1114 results in data 1118, representing the physical location and number of all helper T-cells and monocytes (i.e., CD4+ cells) in the same field of view. The Image 2 (Data 1116) cells are subtracted from the Image 3 (Data 1118) cells in a step 1120 to produce data 1122, identifying the CD4+ T helper cells in the image. With the known field of view sample volume (image field of view area times chamber height as describe above), the CD4+ T helper cell count can be converted to the cells per microliter in the device. With the known volume dilution performed prior adding prepared sample to the device, the CD4+ T helper cell concentration (cells per microliter) in the original blood sample can be calculated (Data 1122).

Assay Protocol Simplification

In one embodiment, an important application of the present invention is rapid, inexpensive identification and enumeration of CD4 T helper cells in whole blood samples at the point-of-care with minimally trained staff operating the instrument. For this application, capillary (finger stick) whole blood is the desired sample matrix. Finger stick specimens present challenges due to the need for accurate volumetric transfer of small blood volumes (e.g., <50 microliters). In one embodiment, micro-tubes and 10 microliter disposable transfer pipettes are prepared with dried anticoagulant (EDTA). Blood volumes of 10 microliters or less are desirable because larger finger stick volumes (e.g, 20 to 50 microliters) are more difficult to obtain, requiring larger lancets and more extensive finger manipulations. In another aspect, “milking” the finger to increase the volume may lead to significant amounts of interstitial fluid in the specimen. Dilution with interstitial fluid creates counting errors in a quantitative CD4 measurement. Devices that handle 10 microliters or less sample volume may help improve the accuracy of the assay, and are used as demonstration here.

In another embodiment, assay reagents (stains and lysis reagents) are dried in the cartridge or in the sample handling device. Dried, on-board reagents provide improved environmental stability relative to liquid reagents. Preparation of dried reagents can be by lyophilization, either directly into cartridge, sample handling device, or volumetric transfer device components, or as pellets added to the cartridge, sample handling device, or volumetric transfer device.

In another embodiment, rehydration of dried assay reagents is with the sample. In another embodiment, rehydration of dried assay reagents is with water or buffer added at the time of assay. In another embodiment, rehydration buffer or liquid assay reagents are stored on-cartridge, for example, in a blister pack. In an embodiment, a manual action such as closing a cap or squeezing a section of the cartridge releases rehydration buffer or liquid assay reagents.

In another embodiment a combination of on-cartridge stored liquid reagents (e.g., blister packs) and a rack with active features may be implemented. In an embodiment, the cartridge “opening” may include, for example, a 10 microliter capillary tube to draw the finger stick or venipuncture specimen into the device. As described above, dried stain and EDTA, and potentially lysis salts, can be coated on the tube walls. After the specimen is added, the operator may close a cover over the capillary tube, causing a plug or plunger to interface with the capillary tube and push the 10 microliter sample into an interior channel of the cartridge. The cartridge may be placed on rack where a mechanical actuator may press on-cartridge blister pack after a pre-defined incubation period. The blister pack may contain rehydration buffer to rehydrate on-board dried reagents. Or the blister pack may contain liquid assay reagents. In another embodiment, on-cartridge wash buffers may be deployed manually or via the rack actuator.

In an embodiment, direct imaging of neat whole blood without dilution or RBC lysis is performed. This embodiment provides the advantage of eliminating the addition of liquid reagents. In one embodiment, channel height is reduced to create a thinner layer of cells in the imaging field of view. Channel height is selected to minimize interference of RBC background.

In another embodiment, the brightfield images are not used in the analysis. In another embodiment, RBCs are removed by agglutination (e.g., analogous to a Coomb's test).

In an embodiment, printed controls are provided on a surface of the cartridge. For example, relevant printed controls may include printed spots of the cell markers, CD4 and CD14, which would function as procedural controls for fluid flow and function of the anti-CD4 and anti-CD14 antibody stains. Printed peripheral blood mononuclear cell (PBMC) preparations dried by methods such as those found in U.S. Pat. No. 6,008,052 could also serve as procedural controls. In addition, printed anti-hemoglobin antibody spots would serve to verify RBC lysis and the addition of blood. The captured hemoglobin would be detected by a fluor-labeled anti-hemoglobin antibody that was included in the dried protein mixture with the CD4 and CD14 stains or in the wash solution. To avoid false positives caused by minor amounts of intrinsic hemoglobin in the blood, the amount of printed antibody and concentration of the labeled anti-hemoglobin antibody may be adjusted to scale around the proper range of lysed RBC's.

Adjustable pipettes are considered “complex” from an FDA/CLIA perspective and are often unavailable in limited-resource settings. Disposable transfer devices have therefore been characterized for use in an assay kit, in accordance with an embodiment. Devices were obtained from Poly-Pipets Inc. (Englewood Cliffs, N.J.) and SafeTec Clinical Products (Ivyland, Pa.). The Microsafe tubes from SafeTec are in clinical use in approved tests such as the Hemosense INRatio products. Accuracy and reproducibility experiments suggest that the Microsafe products have acceptable accuracy.

Furthermore, regardless of the Clinical Laboratory Improvement Amendments (“CLIA”) waive status of a particular test, certified clinical laboratories regularly, often daily, perform QC checks with negative and positive control solutions. In an embodiment, microparticles such as latex beads (0.3 to 10 micrometer diameter) with or without CD4 and CD14 markers on their surface may serve as reference controls.

In an embodiment, fluorescent beads as calibrators for inconsistent volumes of lysis solution may be used. A fixed number of fluorescent beads may be added to the dried reagent mix at the beginning of the assay, resulting in the beads being imaged with the cells. The measured bead counts may be compared to the expected bead count, and the difference would reflect the actual amount of added lysis solution. The use of fluorescent beads for count calibration is common in flow cytometry, with the CountBright (Invitrogen) and CytoCount (Dako) beads being examples. The added beads would not necessarily interfere with the targeted CD4, or helper T cell count, because the bead fluorescence would match the emission of the anti-CD14 label, i.e., the red channel, and the bead counts would therefore be subtracted in the standard image algorithm. The density of the calibration beads may be adjusted to provide imaged bead counts that are accurate, but not overwhelming.

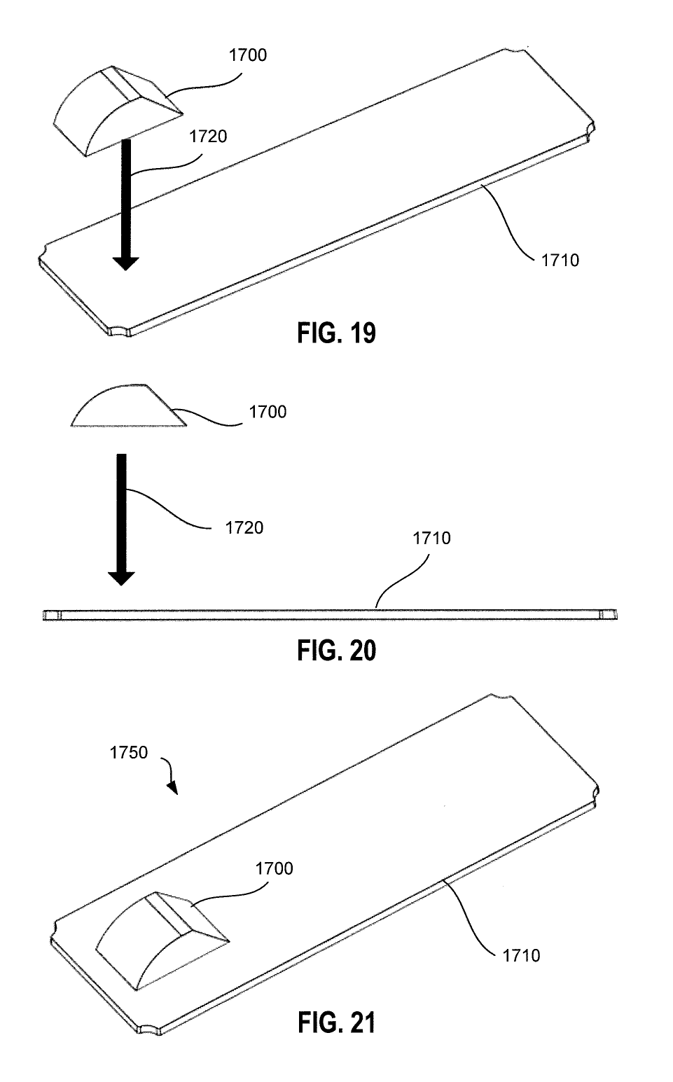

FIGS. 19-21 illustrate an alternative way of forming a planar waveguide suitable for use with the embodiments described herein. FIG. 19 shows a perspective view of a refractive volume 1700 and a planar waveguide 1710, being brought together as indicated by an arrow 1720. FIG. 20 shows a side view of refractive volume 1700 and planar waveguide 1710. FIG. 21 shows a planar waveguide arrangement 1750, in which refractive volume 1700 and planar waveguide 1710 are combined as a single component.

As shown in FIGS. 19-21, refractive volume 1700 may be separately formed from planar waveguide 1710, then brought together (as indicated by arrow 1720) to form planar waveguide arrangement 1750. Planar waveguide arrangement 1750 may be used to replace, for example, planar waveguide 1005 of FIG. 12. Separate fabrication of refractive volume 1700 and planar waveguide 1710 may be advantageous from a manufacturing perspective. For example, an index-matching liquid or epoxy may be used to bond together refractive volume 1700 with planar waveguide 1710 so as to simulate the performance of an integrally-formed planar waveguide, such as planar waveguide 1005 of FIG. 12.

The following examples are provided for purposes of illustration of embodiments only and are not intended to be limiting. The reagents, chemicals and other materials are presented as exemplary components or reagents, and various modifications may be made in view of the foregoing discussion within the scope of this disclosure. Unless otherwise specified in this disclosure, components, reagents, protocol, and other methods used in the system and the assays, as described in the Examples, are for the purpose of illustration only. A large number of antibodies of varied isotypes have been investigated, as well as stains for CD4, CD14, CD3, CD8, and CD45. These experiments successfully demonstrated our ability to look at multiple markers. The examples below demonstrate the measurement of absolute cell counts using CD4 and CD14, although other combinations of markers may be used to provide absolute cell counts, within the context of the present disclosure.

### EXAMPLE 1

### CD4 T Helper Cell Count from 10 Microliters of Whole Blood Sample

Biological Reagents. For the purposes of this disclosure, commercially available and commonly used antibodies were used. However, it is to be recognized that other commercial or customized antibodies may also be used. It is also to be understood that for any given antibody, a single batch or a mixture of antibodies directed against the same antigen may be used. Such antibodies may be obtained from different sources but are all reactive against the same antigen.

Assay Reagents. Some of the assay reagents include bovine serum albumin (“BSA”, Sigma Life Science, St. Louis, Mo.), phosphate buffered saline (“PBS”, Fisher Scientific, Rockford, Ill.).

Blood Samples. Because one of the important uses of the instant system and method will be in point-of-care settings, it is important to evaluate the performance of the system on whole blood samples. Whole blood was sourced under an IRB-approved protocol from HIV-positive donors at a U.S. research university. Venipuncture samples were collected in Ethylenediaminetetraacetic Acid (“EDTA”) blood collection tubes (Lavender Cap BD Vacutainer®) and shipped overnight to the site where the assays were run within two hours of receipt of the samples (i.e., within 24 hours of draw).

Assay Cartridge and Instrument. The system described in the examples here combined single-use disposable assay cartridges with a reader instrument. Fluorescence immunoassays were illuminated and imaged using a multi-mode planar waveguide technology. Various types of planar waveguides have been used in biosensor and immunoassay applications for decades, and are the subject of several technical reviews. Briefly, a light source (typically a laser) was directed into a waveguide substrate. The present system uses a planar waveguide system as disclosed, for example, in U.S. patent application Ser. No. 12/617,535 entitled “Waveguide with Integrated Lens” as filed 12 Nov. 2009, and U.S. patent application Ser. No. 12/942,234 entitled “Planar Optical Waveguide with Core of Low-Index-of-Refraction Interrogation Medium” as filed 9 Nov. 2010, which applications are incorporated herein by reference in its entirety.

The reader instrument design incorporates two laser diodes (540 and 635nm) and a brightfield LED with simple imaging optics. In an exemplary embodiment, these illumination sources may be low-cost, stock lasers and LEDs with no moving parts in the three-mode illumination system. The imaging optics includes collection optics for imaging light signal at the attachment surface of the cartridge onto a low-cost CMOS detector.

A simple mechanical translation module has been incorporated into the reader instrument, thus allowing imaging of nine adjacent fields of view (“FOV”) across the sample volume. The increased number of FOV improves count statistics, particularly for low CD4 count samples. With nine FOV, the resulting statistical count error (%CV) at 200 cells/microliters has been reduced to approximately 10% for the nine FOVs relative to 30% for a single field of view. The autofocus mechanism, described in FIGS. 9-11, has also been implemented in the present Example. The autofocus mechanism may be based, for example, on a stepper motor module, and other implementation schemes may also be contemplated. We note that the main purpose of the autofocus is fine focus adjustment. In an exemplary embodiment, the planarity of the waveguide and cartridge combined with mechanical tolerance of the reader components results in only small focus adjustments being required during cartridge imaging.

The cartridge used in the Examples is a simple single channel assembly based on an injection-molded, plastic planar waveguide (e.g., first substrate 304 of FIG. 3) with an integrated lens (e.g., integrated lens 1010 of FIG. 12) to facilitate light insertion therein. A double-sided adhesive gasket is used to define a fluidic channel for containment of the processed sample. The gasket also binds the planar waveguide to an injection molded upper component (e.g., second substrate 306 of FIG. 3). The upper component provides fluid input and output ports, as discussed in U.S. Provisional Patent App. Ser. No. 61/469,954, filed 31 Mar. 2011 and entitled “Cartridge for Use with a Reader,” which is incorporated herein in its entirety. An absorbent pad above the output port on the upper component may be enclosed with a snap-in plastic lid, making cartridge fluids self-contained, thereby minimizing biohazard. Laser welding may further provide a lower cost, potentially more rapid alternative to gasket adhesion.

Prior to assembly into the cartridge, the inner surface of the planar waveguide is activated with a cationic polymer layer to provide “universal” white blood cell adhesion (i.e., an attachment surface, as previously discussed). For example, the attachment surface may be treated with poly-L-lysine (“PLL”) or any other suitable surface modification such that the modification enhances the affinity between the attachment surface and the analyte or object, such as a mammalian cell. As the sample flows through the narrow volume between the first inner surface and the second inner surface, one or more of the analytes or objects in the sample may settle and/or attach to the attachment surface.

Samples may be processed before and after loading onto the cartridges on benchtop at ambient temperature, which in this study was approximately 20 to 25° C. Since the assay procedure may be performed independently of the reader instrument, sample cartridges can be batch processed, with up to 30 run in parallel, for example. Sample volumes for the cartridge are about 10 microliters of whole blood, making the cartridge compatible with finger stick capillary samples.

As discussed previously, image processing is performed on the images captured at the reader instrument for spot finding, counting, intra-spot fluorescence signal intensity measurement and normalization, monocytes signal subtraction and debris staining subtraction. After desired data have been collected, the cartridge may be removed from the reader instrument and disposed as biohazard waste, and the next processed cartridge may be inserted into the reader instrument.

### Protocols and Results

The assay protocol, as illustrated in FIG. 6, was performed to generate the data set for this example. Blood samples were received and processed within 24 hours of blood draw. Briefly, a 10-microliter fresh whole blood sample was stained with dye labeled anti-CD4 and anti-CD14 antibodies (R-phycoerythrin (“R-PE”) and Alexa649, respectively) for 5 minutes. 6 microliters of stain reagent were used in this step. The sample-stain mixture was then added to 100 microliters of ammonium chloride RBC lysis buffer. At this point, sample dilutions were known and may be used to back-calculate counts in the original blood sample. After 5 minutes of lysis, approximately 40 microliters were transferred to the inlet port of the cartridge, and the cartridge channel rapidly filled by capillary action. Fill time is typically less than 5 seconds, at which point flow stops. Note that an accurate volume is not required on this transfer step. Gravity sedimentation of WBCs to the polymer attachment surface was complete within approximately 5 minutes of sample addition to the cartridge. Finally, a wash solution was added to flush excess fluorescent stain through the channel. The cartridge was then inserted into the reader instrument, where image capture and data processing was completed in approximately 3 minutes. Eight to twenty cartridges can be processed in parallel and sequentially imaged, resulting in a throughput of up to 20 cartridges per hour.

In the reader instrument, laser light was coupled into the optical waveguide to illuminate the stained cells, and the captured fluorescent images were analyzed to distinguish the different cell types and tally the numbers of the different cell types, using the method described in FIGS. 8, 10, 11, 13 and 14. Image analysis was performed to compare both fluorescence and brightfield images in order to resolve debris, unlabeled cells, and cells with either or both stains. As discussed in detail above, both CD4 T cells and monocytes are stained by the dye-labeled anti-CD4 antibody. By contrast, only monocytes are stained by the dye-labeled anti-CD14 antibody. Thus, the total number of CD4 T cells generally equals the number of anti-CD4 staining cells minus the number of anti-CD14 staining cells, with adjustment from the brightfield results.

In particular, images were analyzed to count helper T cells specifically by correlating three overlapping images in each field of view. As previously described, brightfield images are used to discriminate non-cellular debris from actual cells. In the present example, fluorescent objects in the 635 nm laser (red) image are stained with anti-CD14 antibodies and are monocytes (i.e., Image 2 of FIG. 13). Fluorescent objects in the 540 nm laser (green) image are stained with anti-CD4 antibodies and may be either helper T cells or monocytes (i.e., Image 3 of FIG. 13). By subtracting red objects from green objects in the registered image stack, an absolute helper T cell count is generated (referred to as CD4 count). Multiple differential staining approaches (e.g., CD4/CD8, CD4/CD3) may be utilized in place of the CD4/CD14 combination discussed in the present example.

Over 135 clinical specimens have been processed using the present system and methods and the results were compared to the results obtained using flow cytometry. Multiple commercially available stain antibodies at varied concentrations and mixtures have been tested to optimize performance.

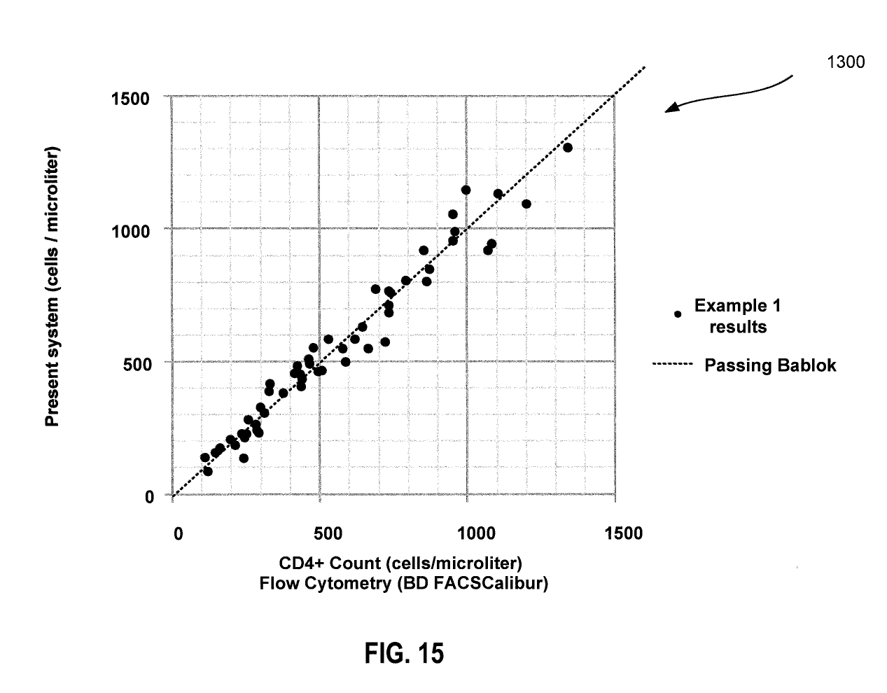

FIG. 15 shows a comparison study of the results obtained from whole blood sample and CD4 depleted blood sample as measured by the instantly disclosed system and by standard FACS methods, respectively. De-identified whole blood samples were split and processed same day at two facilities, one at the University of Colorado Denver Health Science Center using a BD FACSCalibur™ (Dual Platform). The other was performed using the system and methods disclosed herein. Each circle in the plot represents one whole blood sample run on both systems. Perfect agreement would yield a straight line along the diagonal “identity” line. Passing-Bablok regression (dashed line) shows a slope deviation less than +/−5% at 95% confidence and an intercept deviation less than 15 at 95% confidence. As shown in FIG. 15, the CD4 T cell counts obtained from the “gold-standard” FACS and those obtained using the presently disclosed system and methods are comparable.

While a variety of antibodies, buffer solution concentrations and incubation times may be suitable for use in the embodiments described herein, one example of possible combination of antibodies is OKT4 (Biolegend), SK3 (BD), and 13B8.2 (Beckman Coulter) as the anti-CD4 antibodies, and HCD14 (Biolegend) and TUK4 (AbD Serotec) anti-CD14 antibodies. Although it is recognized that OKT4 is not effective for a particular gene variant that is present in approximately 8% of the African population, cocktail mixtures of OKT4 and either SK3 or 13B8.2 may help avoid this potential error and have also been observed to provide more intense staining, likely due to binding at different epitopes.

### EXAMPLE 2

### CD4 T Helper Cell Count from 10 Microliters of Whole Blood Sample Using a Combined Stain and Lysis Protocol

200 microliters of 10×RBC Lysis Buffer (Biolegend Catalogue #420301) were mixed with 1.8 ml deionized water to prepare 1× RBC Lysis Buffer. The 1×RBC Lysis Buffer was allowed to warm to room temperature. Cartridges were removed from packages and placed on a level surface. An antibody cocktail was prepared by mixing the following three antibodies: (a) Mouse anti-human CD4 Phycoerythrin, clone OKT4, (Biolegend Catalogue #317410); (b) Mouse anti-human CD4 Phycoerythrin, clone 13B8.2 (Coulter, Catalogue #IM0449U); and (c) Mouse anti-human CD14 AlexaFluor 647, clone HCD14 (Biolegend, Catalogue #32561). For example, the antibodies may be mixed by volume as 1 microliter of OKT4, 2 microliters of 13B8.2, and 1 microliter of HCD14; the exact mixture may be optimized according to assay performance. 4 microliters of the antibody cocktail were removed after vigorously mixing the cocktail by vortexing. The 4 microliters of the antibody cocktail were added to 100 microliters of 1×RBC lysis buffer in a sample tube and mixed well.

The 2-step sample processing protocol as described in FIG. 5 was performed to generate the data set in this example. Blood sample in a Vacutainer® was mixed well by repeated tube inversion immediately before 10 microliters of the blood sample was removed and added to the sample tube containing about 104 microliters of the antibody cocktail/lysis buffer mixture as described in the immediately preceding paragraph.

The blood sample was gently mixed with the antibodies and RBC lysis buffer in the sample tube. “Gentle mixing” has been successfully performed with several methods, including a low speed vortexer, tube inversion, or slow aspirate and dispense with a pipette. 35 microliters of the mixture was then loaded to the inlet port of a cartridge. The fluidic channel of the cartridge fills by capillary action, and the loaded cartridge was allowed to sit at a level surface for about 20 minutes before 200 microliters of a wash was added into the inlet port to fix the cells and wash away any unattached dye/fluorescence stain. It is noted that the narrow height of the fluidics channel and high surface exposure of the cells to the solution removes the need to rotate or mix the cells during incubation.

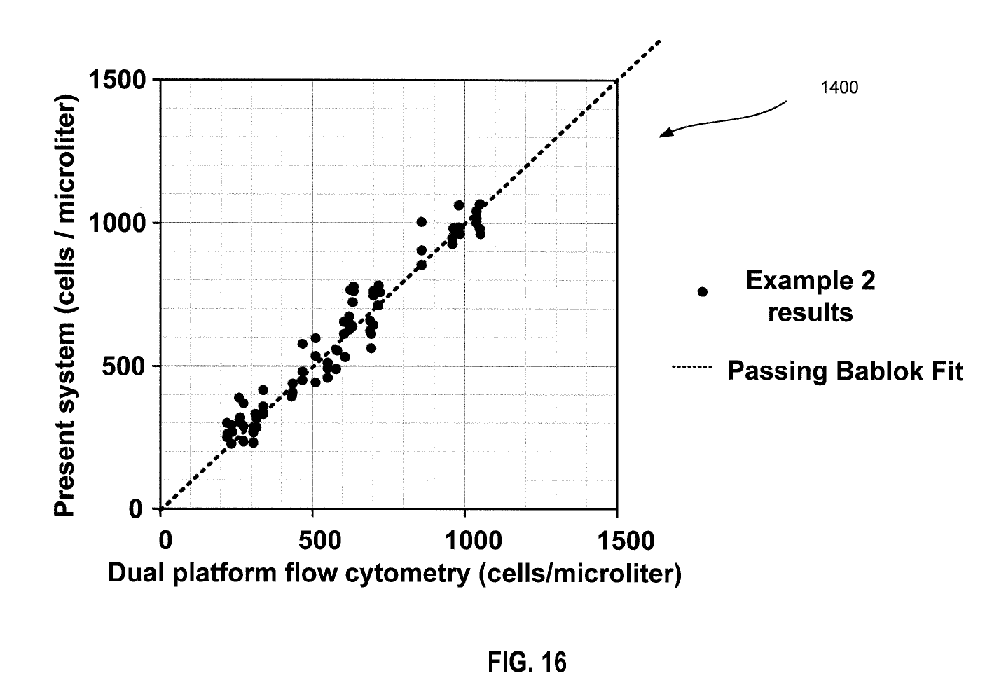

FIG. 16 shows the results of a comparative study comparing the CD4 cell counts obtained in Example 2 and a dual platform, flow cytometry system, quantifying the comparison by showing the distribution of counts with respect to a Passing-Bablok fit.

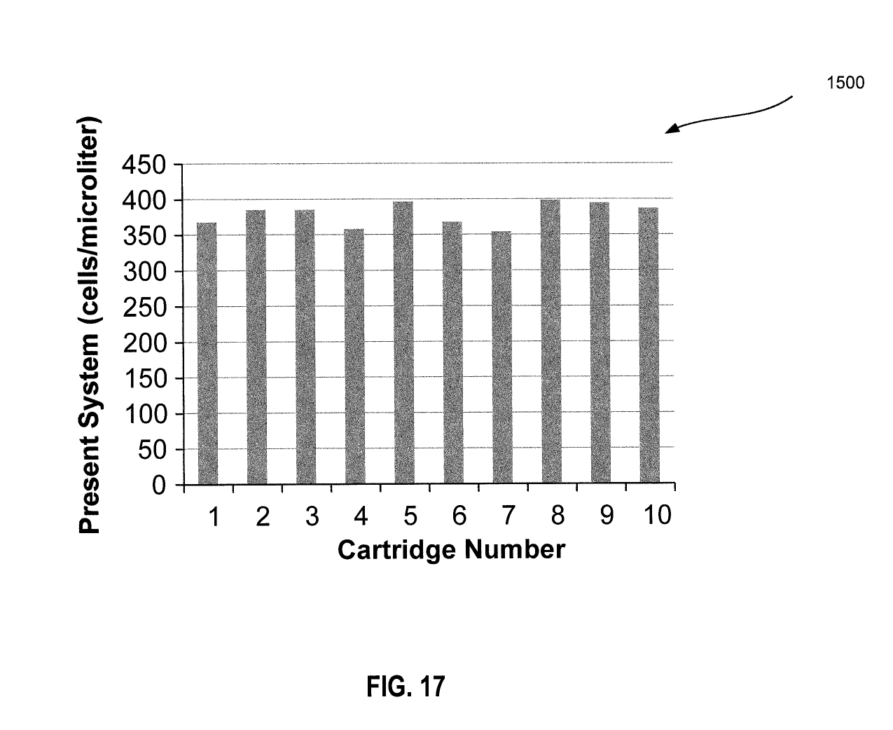

FIG. 17 shows the results of a reproducibility study, quantifying the variation in CD4 cell counts obtained using the protocol of Example 2. Aliquots from a single blood sample were run on 10 consecutive cartridges. The staining protocol was run independently for each of the 10 cartridges.

### EXAMPLE 3

### Preparation and Use of Dried Reagents

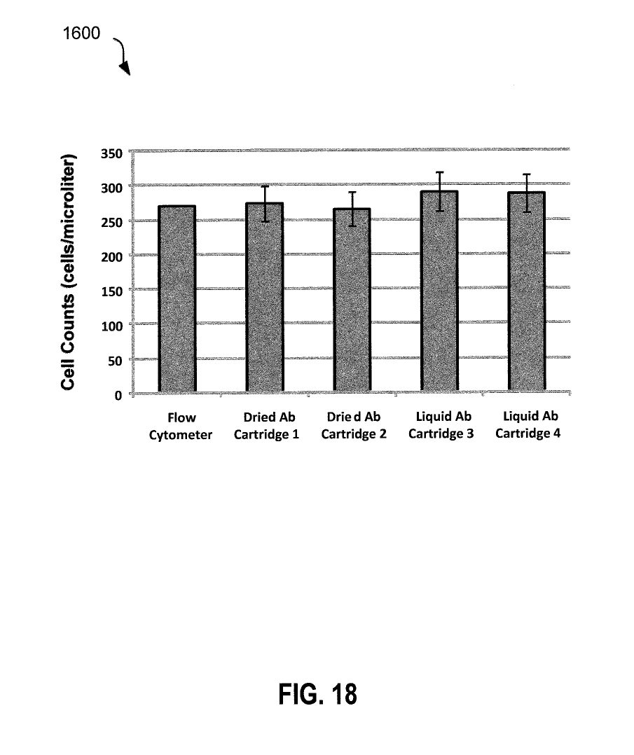

For a point-of-care device, it would be desirable to offer kit components that do not require special controlled temperature storage. Dried kit reagent development is described briefly here. The most labile reagents in the presently described protocol may be the stain solutions, especially when stored at room temperature in facilities that may have inconsistent temperature control. Antibody stains in sample collection tubes have been successfully dried and assays with performance equal to direct addition of the liquid stains and flow cytometry measurements have been repeatedly demonstrated. Further, EDTA was incorporated into the dried antibody spot, resulting in direct anti-coagulant addition and staining of the 10 microliters blood sample. Experiments indicate high performance with only 5 to 10 minutes of incubation. These drying experiments have been based on selection of an appropriate sugar-based solution and overnight vacuum drying. Higher antibody activity may be achievable with lyophilization. FIG. 18 shows cell count comparisons across flow cytometry (FACSCaliber) and with dried or liquid antibodies with presently described cell counting system. The measurements using the presently described cell counting system are from individual cartridges as indicated by 1 or 2. The error bars are based on the number of cells counted across multiple fields of view in each cartridge. The error in the flow cytometry measurement is not known.

Examples 1 and 2 and experiments thus far have been conducted using a typical ammonium chloride lysis buffer (the standard formulation includes ammonium chloride (150 mM NH₄Cl), potassium bicarbonate (KHCO₃), and EDTA.) A potential disadvantage of a kit containing NH₄Cl lysis buffer is the instability of a 1× solution. Internal studies suggest a 50% loss of lysis activity in 2 weeks. To integrate the combined stain/lysis step into the assay and avoid the instability of a 1× solution, the anticoagulant, stain, and lysis salts may be combined into a single dried pellet. The addition of blood sample would provide the initial liquid to solubilize the reagents. Similarly, a dried lysis package of NH₄Cl and KHCO₃ may also be feasible either by drying a prepared solution or mixing the dried chemicals of ammonium chloride and potassium bicarbonate. To prevent concentrated and extended exposure of the antibodies to the NH₄Cl solution, separate pellets of stain and lysis buffer may be prepared and placed in the same sample tube, and, if necessary, pellets of stain and lysis solution can be further separated into foil “pouches.”

A dried lysis/stain pellet or pellets may not completely eliminate the need to add liquid for dilution. For example, excess densities of RBC's, even lysed RBC debris, may obscure the cell images and a diluent, such as deionized water may need to be added to achieve the proper dilution.

### EXAMPLE 4

### Use of a No-Wash Protocol

The wash buffer serves to remove the background fluorescence of unbound stain in the bulk solution, resulting in fluorescent cells that are more clearly resolved. Wash buffers may be either an isoosmotic rinse or a fixative solution like paraformaldehyde. Although the wash does improve resolution of the cells and is analogous to the centrifugation steps used in flow cytometry, it may be desirable to eliminate this wash step. That is, without the wash step and assuming reasonable expectations for dried stains and lysis reagents, the assay protocol may become as simple as an initial mixing of whole blood, dried reagents, and a premeasured “vial” of deionized water that is dispensed into the cartridge. For instance, after an incubation time of 10 to 30 minutes, the cartridge (or set of cartridges) may be immediately inserted into the reader instrument.

A preliminary experiment to image stained and lysed sample preparations without the wash step has been performed. In this experimental set, stain concentrations and times were varied, and as expected, lower stain concentrations had lower backgrounds and required longer times for equivalent stain intensities. The most striking and relevant feature of these results is that images without a wash step do allow the stained cells to be resolved over the fluorescent background.

### EXAMPLE 5

### Preparation and Use of a Combined Lysis and Staining Reagents

Although the procedure described in Example 2 combines staining and red blood cell (RBC) lysis into one step, it requires an operator to mix the staining solution and the lysis buffer shortly (usually within a few hours) before the staining-lysis mixture is to be mixed with the blood sample. This extra reagent preparation step may be an extra burden for the operators, and may increase the complexity of the process, which, in turn, may cause more potential errors in the assay.

It is disclosed here a combined reagent containing both the staining reagent and the RBC lysis reagent in one stable formulation. The stable formulation provides a single stock reagent for testing without additional reagent preparation steps by the operators. This improvement may simplify the process of detecting specific lymphocytes in whole blood and may render the disclosed assays more compatible with CLIA waived procedures and more suitable for an expanded global market. In one embodiment, the steps of cell staining and red blood cell (RBC) lysis may be combined into one step with a single liquid reagent. The whole blood specimen is added to the stain & lysis solution before the blood/stain/lysis mixture is added to the cartridge.

In another embodiment, ammonium oxalate (11.45 mg/mL) may be used in place of ammonium chloride because ammonium oxalate has been demonstrated to have extended room temperature stability. In another embodiment, the antibody stains may be mixed and stored with the ammonium oxalate solution as a pre-measured, ready-to-use reagent. Bovine serum albumin (BSA) concentrations from 0 to 25 mg/mL may be added to the combined stain & lysis solution, and ProClin and sodium azide may be used as antimicrobials. This combined lysis and stain solution has shown at least 2-month stability at room temperature.

In this example, the assay protocol is significantly improved from an operator workflow perspective. Ready-to-use stain/lysis solution microtubes (sample handling device) are supplied to the user. Prefilled ammonium oxalate/stain solution tubes are supplied to the user. A 10 microliter blood sample is transferred directly to the tube using a controlled volume transfer pipet (e.g., Micro-Safe®). The sample/reagent solution is gently mixed and then transferred to the cartridge for the counting assay. Smaller volumes of whole blood: 3 and 5 microliters, have also been successfully used with proportional changes in the volume of the stain & lysis solution.

### EXAMPLE 6

### Planar Optical Waveguide With Core Of Low-Index-Of-Refraction Interrogation Medium

The following example describes a planar waveguide that may be used in the device and system disclosed herein. Fluorescently labeled probes provide a convenient method of characterizing the content of biological samples. By tailoring the binding chemistry of a fluorescent probe, high specificity can be achieved for detection of complex molecules such as RNA, DNA, proteins, and cellular structures. Since fluorophores typically absorb and re-emit Stokes-shifted radiation regardless of being bound or unbound to a species to be detected, the bound and unbound fluorophores typically need to be separated.

One common method to separate the bound fluorophores from the unbound fluorophores relies on spatial localization of the fluorescently labeled species. For example, in a ‘sandwich immunoassay,’ a surface is chemically treated to bind a species to be detected to that surface. The fluorescent probes then attach to the species that are bound to the surface. Unbound fluorophores can then be removed from the system with a wash step.

Background fluorescence can be further reduced if the excitation light can be confined to the surface. Total internal reflection fluorescence (“TIRF”) is one method of reducing background fluorescence. In general, when light propagates from one medium to another, a portion of the light will be reflected at the interface. If the light is propagating into a material with a lower index of optical refraction, however, all of the light will be reflected if the angle at which the beam is incident on the surface is greater than the ‘critical angle’ (relative to the surface normal). In the lower index material, the light intensity exponentially decays with distance from the surface. This exponentially decaying field (known as an ‘evanescent field’) has a characteristic decay length on the order of 100 nanometers to 1 micrometer for visible light. The light of the evanescent field will, therefore, only excite fluorophores that are localized at the surface.

In a simplified implementation, TIRF is performed with a laser beam reflecting once from the surface. This is the basis of well established TIRF microscopy and other biosensing techniques. By confining the laser beam inside a waveguide, however, multiple reflections can be realized and larger areas can be illuminated. Several waveguide geometries are possible, each having certain tradeoffs.

Single-mode planar waveguides, also called thin film waveguides or integrated optical waveguides, confine light into a small cross sectional area with the thin dimension smaller than the wavelength of propagating light. The advantage of single-mode waveguides is that significantly stronger evanescent fields are generated. A disadvantage of single-mode waveguides is that for efficient light coupling, they typically require a prism or grating with precise alignment tolerances. In addition, single-mode planar waveguides are expensive to manufacture because the guiding layer is typically a thin-film with strict thickness tolerances deposited on a substrate. In contrast, a multimode planar waveguide is substantially easier to couple a laser beam to and simpler to construct than single-mode planar waveguides. For example, a standard 1 millimeter thick microscope slide makes an effective waveguide into which light can be coupled through the edge of the slide. Additionally, dimensions for multimode waveguides are compatible with current plastic injection-molding techniques.

For a fluorescence-based assay system, a uniform evanescent field is desired in the detection region. By definition, the strength of the evanescent field is uniform along the direction of light propagation for a single-mode planar waveguide (neglecting scattering losses and absorption inside the waveguide). For a disposable clinical device, however, cost, robustness, and ease of use are of similar importance. By adjusting input coupling to a multimode waveguide, uniformity and field strength of the evanescent field can be optimized.

While each individual mode in a multimode waveguide has a uniform intensity along the direction of propagation, a distribution of modes will be excited when coupling to a multimode waveguide; this distribution of modes will constructively and destructively interfere on the surface and lead to a spatially varying field strength. When the thickness of the waveguide is much larger than the wavelength of light, the mode structure of the waveguide can be neglected, and the intensity in the waveguide can be treated as a conventional diffracting beam that totally-internally reflects from the two surfaces of the waveguide and interferes with neighboring reflections.

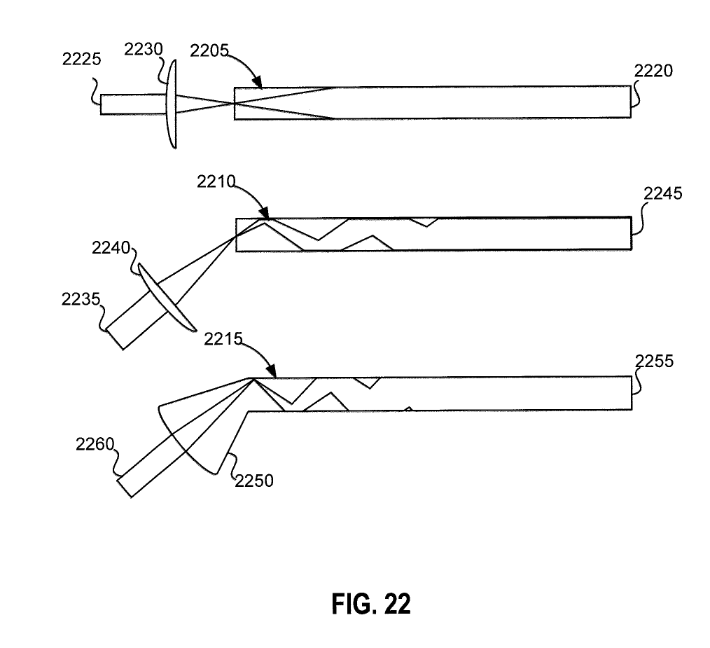

FIG. 22 illustrates several examples of existing coupling schemes 2205-2215 involving multimode waveguides. Coupling scheme 2205 using a multimode waveguide 2220 involves focusing a laser beam 2225 that propagates parallel to a waveguide 2220 into the edge of waveguide 2220 with a cylindrical lens 2230. The field strength of a total internal reflection (“TIR”) beam, however, is maximized for a beam that is incident at the critical angle and zero for a beam with an incident angle 90° from the surface normal (i.e., grazing incidence). Thus, an incident beam that is parallel to the TIR surface will have small evanescent field strength when coupled to waveguide 2220 with cylindrical lens 2230 in the configuration of the scheme 2205.

A variation on coupling scheme 2205 is illustrated by coupling scheme 2210. In coupling scheme 2210, a laser beam 2235 focused by a cylindrical lens 2240 is incident on the edge of a waveguide 2245 with an appropriate angle such that a central ray of laser beam 2235 inside the waveguide impinges on the surface near the critical angle for TIR to maximize the evanescent field strength. A compromise between field strength and uniformity may be made by the choice of focusing optics. If a nearly collimated beam is used to achieve high field intensity by operating near the critical angle for TIR, the beam must make many reflections within the waveguide before the surface intensity becomes sufficiently uniform, thus requiring a longer waveguide. If the beam is highly focused, however, then the surface intensity normalizes in very few reflections, but a significant amount of power is contained in rays propagating outside the critical angle and leads to reduced evanescent field strength down the length of the waveguide.

Precise alignment of a cylindrical lens, such as lenses 2230 and 2240, relative to the input face of a waveguide, such as waveguides 2220 and 2245, respectively, must be made in order to have a laser beam focused on the input face. One proposed solution to this problem is illustrated by a coupling scheme 2215. In coupling scheme 2215, a lens 2250 is incorporated with a waveguide 2255 as a single optical component, made, for example, by bonding the lens element to the planar waveguide or by molding a single optical component. While this allows the focus of lens 2250 to be precisely distanced from the edge of waveguide 2255, careful alignment of a laser beam 2260 relative to lens 2250 of waveguide 2255 must still be made to couple beam 2260 to waveguide 2255. For applications requiring repeated placement of a waveguide component relative to the light source, it is highly desirable for the light coupling to be relatively insensitive to misalignment.

In practical applications, the penetration depth of the evanescent field usually is less than a wavelength of the incident light. This aspect is an advantage in some applications, as the evanescent field can serve as a mechanism to illuminate only a volume of interest, e.g., a thin layer in the lower refractive index medium proximate to the waveguide surface. On the other hand, when the object of interest, such as a cell or the bulk of a solution, extends substantially beyond the penetration depth of the evanescent wave, evanescent illumination can be less effective than floodlight-type illumination.

A subfield of integrated optofluidics is concerned with the development of methods for using optical waveguides to illuminate extended liquid media. Most of the developed methods involve the containment of a liquid sample by other liquid and/or solid materials, thereby effectively creating a waveguide for illuminating the liquid sample. Most TIR-based designs involve surrounding the liquid sample with media of lower index of refraction than that of the liquid sample itself. It is then theoretically possible for light to be guided in the liquid sample by TIR at the interface between the high refractive index liquid and the lower refractive index surroundings. However, in practice, waveguiding in a liquid sample contained in another material is difficult due to the fact that common liquids have lower refractive indices than common solids; for example, water has a refractive index of approximately 1.33, while most solid materials have an index of refraction of 1.4 or more. Consequently, a majority of the TIR waveguide designs involve using either high refractive index (i.e., “high-n”) liquids or more exotic low refractive index (i.e., “low-n”) solids.

In interference-based optofluidic waveguides, light is confined to a liquid core by reflection from surrounding materials including two or more layers of higher-index materials combined to result in a lower effective refractive index for the surrounding media. Some interference-based optofluidic waveguides include photonic crystals, such as multiple alternating layers of materials of different indices of refraction

Embodiments disclosed below allow light to be coupled to a planar waveguide providing a strong evanescent field for sample illumination, while eliminating or greatly reducing inadvertent misalignment by a user. The various embodiments further allow facile tuning of the internal propagation angle inside the waveguide, providing simple adjustment of evanescent field strength. Another embodiment also provides apparatus for performing assays involving placement of a fluidic chamber on a planar waveguide in a manner that is insensitive to the optical properties of the chamber.

In an embodiment, apparatus for illuminating a sample for analysis is disclosed. The apparatus includes a light source, a planar waveguide, and a refractive volume. The light source provides light along a propagation vector. The planar waveguide is oriented such that the propagation vector is perpendicular to the normal vector of the planar waveguide and offset from the planar waveguide in a direction parallel to the normal vector of the planar waveguide. The refractive volume, which is positioned proximate to the planar waveguide, optically couples light provided by the light source to the planar waveguide.

Another embodiment sets forth a method for performing sample analysis. Light is provided from a light source along a propagation vector. A refractive volume positioned proximate to a planar waveguide is illuminated with the light. The waveguide is oriented such that the propagation vector is perpendicular to the normal vector of the planar waveguide and offset from the planar waveguide in a direction parallel to the normal vector of the planar waveguide. The light is then coupled to the planar waveguide via the refractive volume.

Apparatus for performing biological assays is disclosed in yet another embodiment. The apparatus includes a light source, a planar waveguide, a refractive volume, and a detector. The light source provides light along a propagation vector. The planar waveguide has a plurality of specific binding molecules bound to a face thereof. The planar waveguide could further have an array of two or more dissimilar specific binding molecules bound to the face thereof. Additionally, the optical axis of the planar waveguide is oriented parallel to the propagation vector and offset from the propagation vector in a direction perpendicular to a face of the planar waveguide. The refractive volume optically couples light provided by the light source to the planar waveguide and is positioned proximate to the planar waveguide. The refractive volume includes at least a section of a plano-convex cylindrical lens. The detector is positioned to detect light emitted from a region proximate to the face of the planar waveguide having the plurality of specific binding molecules bound thereto.

In an embodiment, an apparatus for illuminating a sample includes a planar waveguide. The planar waveguide includes a first substrate, with a first outer surface and a first inner surface, and a second substrate, with a second outer surface and a second inner surface. The first and second inner surfaces of the first and second substrates, respectively, are spaced apart from each other and partly define a volume for confining the sample therein. The apparatus further includes a light source for providing light directed toward the planar waveguide such that the light is optically coupled to and contained within the planar waveguide between the outer surfaces of the first and second substrates, while illuminating at least a portion of the sample contained within the volume.

In a further embodiment, the sample contains at least one object, and the planar waveguide and the light source are configured to cooperate to uniformly illuminate the object. In a still further embodiment, the object is greater than one micrometer in diameter.

In a yet further embodiment, the apparatus further includes a gasket for separating the first and second inner surfaces of the first and second substrates, respectively, while further defining the volume for confining the sample therein. In a further embodiment, the light is contained between the outer surfaces of the first and second substrates at least in part by total internal reflection. In a still further embodiment, the light source provides uncollimated light.

In another embodiment, a sample analysis system includes a planar waveguide. The planar waveguide in turn includes a first substrate, with a first outer surface and a first inner surface, and a second substrate, with a second outer surface and a second inner surface. The first and second inner surfaces of the first and second substrates, respectively, are spaced apart from each other and partly define a volume for confining a sample therein. The sample analysis system further includes a first light source for providing a first illumination directed toward the planar waveguide. The first illumination is optically coupled to and contained within the planar waveguide between the outer surfaces of the first and second substrates while illuminating at least a portion of the sample confined within the volume. The sample analysis system also includes a detector for detecting a first light signal emitted from the sample as a result of the first illumination interacting with the portion of the sample.

In a further embodiment, the sample analysis system includes a second light source, which is configured for providing a second illumination, and imaging optics for directing the second illumination from the second light source to at least another portion of the sample and to the detector. The detector is further configured for detecting a second light signal resulting from the second illumination interacting with the at least another portion of the sample.

Embodiments of the present technology provide for sample illumination such as that involved in fluorescence detection and assay based on evanescent fields using apparatus including a waveguide with an integrated lens. The overall configuration of the apparatus may be such that fluorescence-emitting molecules bound to a waveguide surface are excited by an evanescent field penetrating into the adjacent solution from a light beam propagated within the waveguide, the propagated beam being introduced by an integrally connected lens. The collimated beam of light such as a laser beam may propagate parallel to the waveguide surface such that the system is insensitive to translation of the waveguide. The incident beam may be also appropriately offset from the optical axis of the waveguide such that refraction of the light at the lens surface directs the beam into the waveguide at an angle close to the critical angle for TIR. Additionally, a second integrated cylindrical lens may be added to the output end of the waveguide. This addition of the second integrated cylindrical lens may facilitate a second laser being coupled in the opposite direction, such as for use in multi-color fluorescence assays.

The apparatus may also allow a fluidic chamber to be bound to the planar waveguide such that the chamber contact with the planar waveguide is outside the optical path of the propagating light, eliminating restrictions on optical properties of material comprising the chamber. In some previous configurations, fluidic chambers have utilized low index of refraction materials in contact with the planar waveguide with mechanical clamping in order to limit optical losses at the waveguide/chamber contact area. By separating the waveguide/chamber contact from the optical path, traditional bonding methods such as adhesives or plastic welding may be used to attach the chamber to the waveguide. Moreover, the fluidic chamber may include or be formed in part by a second planar waveguide, wherein the fluidic chamber is disposed between two planar waveguides. In such an arrangement light may be coupled to both planar waveguides as well as the volume formed by the fluidic chamber.

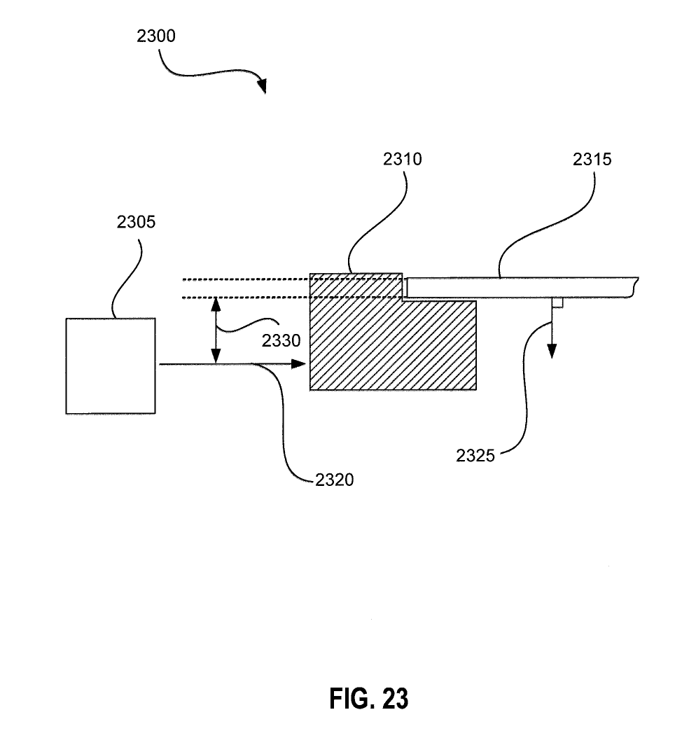

FIG. 23 illustrates a generalized configuration 2300 descriptive of exemplary embodiments. Configuration 2300 includes a light source 2305, a refractive volume 2310, and a planar waveguide 2315. Light source 2305 can include a laser or any other source of collimated or near-collimated light that provides light along a propagation vector 2320. Refractive volume 2310 is positioned proximate to planar waveguide 2315. Refractive volume 2310 and planar waveguide 2315 may lack a discontinuity in index of refraction therebetween. For example, refractive volume 2310 may be adjacent to or abutted to waveguide 2315 with an index matching fluid (not shown) occupying any gap therebetween. Alternatively, refractive volume 2210 may be integrated with planar waveguide 2315 as a single unit or article. Planar waveguide 2315 is oriented such that propagation vector 2320 is perpendicular to normal vector 2325 of planar waveguide 2315. Furthermore, planar waveguide 2315 has an offset 2330 in a direction parallel to the normal vector 2325 of planar waveguide 2315.

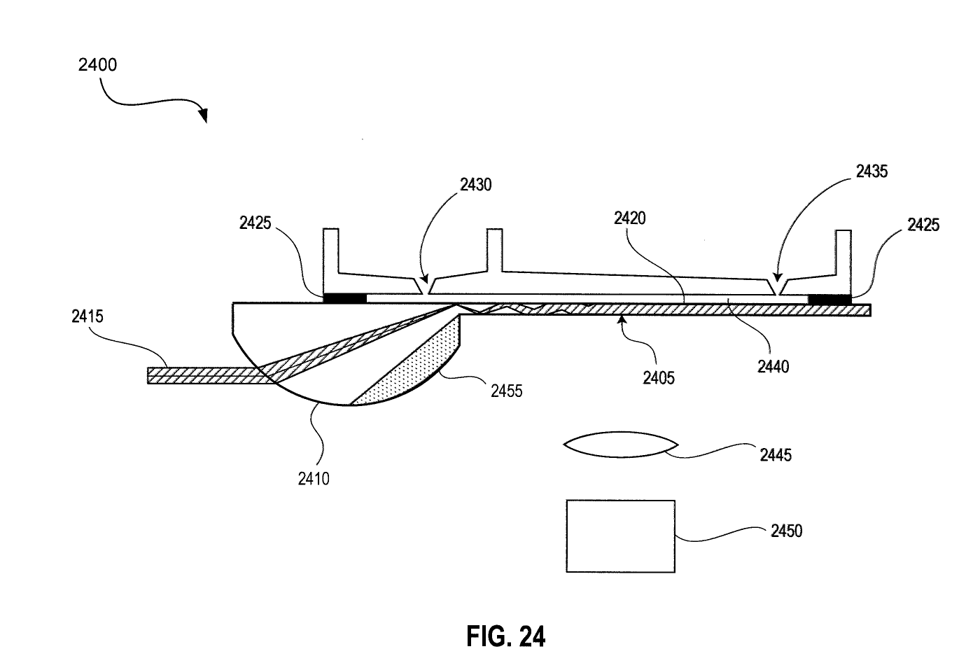

FIG. 24 illustrates an exemplary cross-sectional view 2400 of a waveguide 2405 with an integrated lens 2410 according to one embodiment. Additionally, view 2400 depicts a collimated light beam 2415 such as that of a laser with a wavelength appropriate to excite fluorescent probes at an assay surface 2420. Planar waveguide 2405 with integrated lens 2410 is configured to inject collimated light beam 2415 through a bottom surface of planar waveguide 2405. A flowcell is formed from a sealing mechanism, such as a gasket 2425, an inlet port 2430, an output port 2435, and a fluidic sample chamber 2440, in which chemical compounds deposited on assay surface 2420 of waveguide 2405 may bind the desired target compound to the surface. Collection and filtering optics 2445 can capture fluorescence from assay surface 2420 of waveguide 2405. A signal corresponding to the fluorescence so captured may then be directed to an imaging device 2450 such as a CCD or CMOS camera. Furthermore, the roof, the floor, and/or the walls of the flow cell may be used as a surface on which compounds are deposited.

It is noteworthy that fluidic sample chamber 2440 may include or be formed in part by a second planar waveguide, similar to waveguide 2405, such that fluidic sample chamber 2440 is disposed between two planar waveguides. In such a configuration, light may be coupled to both waveguide 2405 and the second planar waveguide as well as the volume formed by the fluidic sample chamber 2440. The principles described herein are similarly applicable to configurations having multiple planar waveguides.

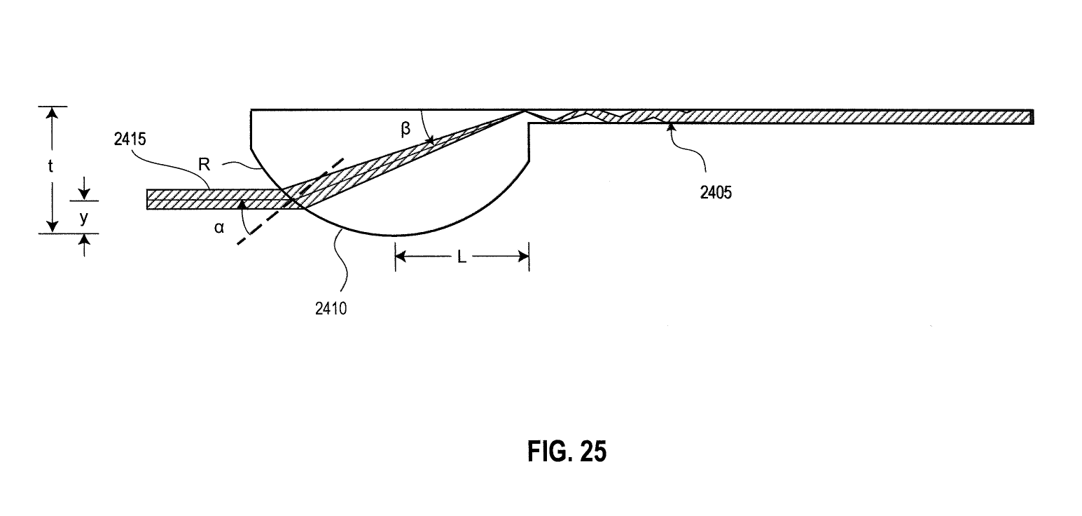

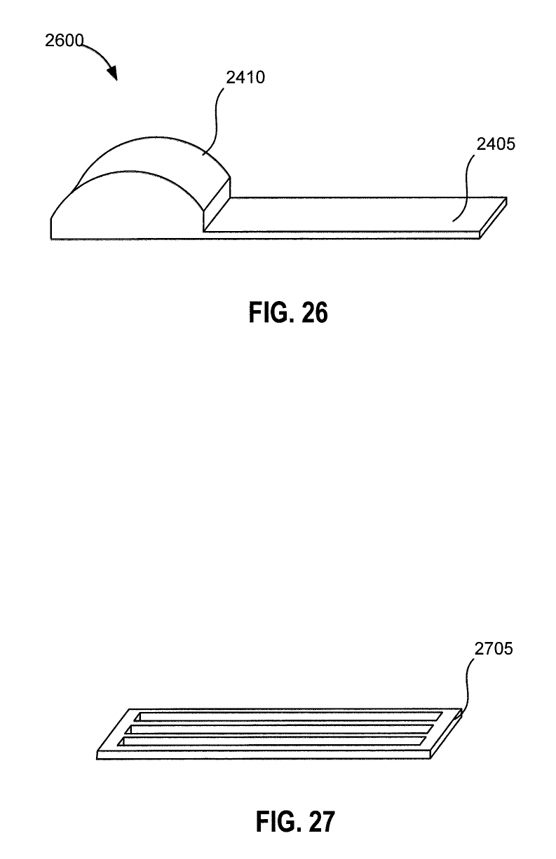

FIG. 25 provides a detailed cross-sectional view 2500 of waveguide 2405 with integrated lens 2410. For further reference, FIG. 26 is a cavalier projection view 2600 illustrating waveguide 2405 with integrated lens 2410. Referring back to FIG. 25, collimated light beam 2415 propagates in a direction parallel or nearly parallel to the optical axis of waveguide 2405, but offset from the optical axis such that it strikes the curved surface of integrated lens 2410. For a clinical instrument in which the waveguide structure is a removable consumable item, this geometry may loosen the positional tolerances necessary to couple collimated light beam 2415 reproducibly to waveguide 2405. Collimated light beam 2415 impinges on the curved surface of integrated lens 2410 at a non-zero angle α relative to the local surface normal of integrated lens 2410, as illustrated in FIG. 25.

As a result of refraction explained by Snell's law, collimated light beam 2415 refracts such that it strikes the top surface of waveguide 2405 at an angle β relative to the optical axis of waveguide 2405. The angle β is defined as the internal propagation angle. The vertical distance y between the center of collimated light beam 2415 and the apex of integrated lens 2410 is chosen such that β is less than the complement of the critical angle allowing total internal reflection to occur. For a given radius R for the curved surface of integrated lens 2410 and index of refraction n for integrated lens 2410, the distance y and angle β are related by the equation:

$$y = R [ 1 - \frac{n sin β}{\sqrt{1 - 2 n cos β + {n}^{2}}} ] . \qquad [ Eq . 1 ]$$

Since collimated light beam 2415 has a spatial extent, the curved surface of integrated lens 2410 will act to focus collimated light beam 2415. The radius R of the curved surface of integrated lens 2410 is chosen such that for a given beam diameter of collimated light beam 2415, the range of angles incident on the top surface of waveguide 2405 is appropriate to provide a uniform evanescent field strength within the detection region while remaining outside the critical angle for TIR. It may be desired that collimated light beam 2415 be focused on the top surface the waveguide 2405 to allow for the greatest tolerance to misalignment. The total thickness t for the structure formed from waveguide 2405 and integrated lens 2410 that leads to a focused beam on the top surface may be given by:

$$t = R + \frac{{( y - R )}^{3}}{{R}^{2} {n}^{2}} . \qquad [ Eq . 2 ]$$

When an appropriate thickness t is used, collimated light beam 2415 will focus at a horizontal distance L from the center of the circle defining the curved surface of integrated lens 2410. L may be related to the previously defined quantities by the equation:

$$L = \frac{t - y}{tan β} - \sqrt{2 y R - {y}^{2}} . \qquad [ Eq . 3 ]$$

The structure including waveguide 2405 and integrated lens 2410 may be manufactured in several different ways. One method is to have the entire assembly constructed in plastic by injection molding technology. An alternative method is to fabricate the planar waveguide and lens element separately from similar index materials. The two elements may then be joined permanently by a transparent optical cement, optical contacting, or temporarily with index matching fluid/oil/gel.

Geometries such as those described in connection with FIG. 24 easily allow the adjustment of the internal propagation angle (β) through a translation, rather than a rotation, of the incident laser beam. This allows for a less complicated mechanical design to couple the laser to the waveguide. Additionally, a new injection molded waveguide is not necessary when it is desired to change the incident angle because the focal point of the lens using the disclosed geometry of FIGS. 24 and 25 is insensitive to the translation of a laser beam relative to the optical axis of waveguide 2405. Further, a desired change in the incident angle is accomplished without changing the readout instrument, allowing variation of cartridge function without physical changes in the instrument. A barcode on the cartridge may be utilized to identify information used to interpret signals from a given cartridge.

To prevent light from leaking from waveguide 2405 after the first reflection from the top surface, cylindrical lens 2410 is truncated such that it does not extend beyond the location of the focus. The area defined by the line connecting the apex of integrated lens 2410 and the point on the bottom surface opposite the focus (see, e.g., optical deadzone 2455 in FIG. 24) will never have light propagate in it that successfully couples to the waveguide. As such, the precise shape of the lens in the area designated optical deadzone 2455 can be any convenient shape provided integrated lens 2410 does not extend beyond the vertical line passing through the focus. For a single injection molded device where minimizing material costs is important, removing all plastic in the area labeled optical deadzone 2455 may be desirable. If two separate components made through conventional optical manufacturing processes are fabricated, integrated lens 2410 that has been diced to remove material beyond the focus can be easily manufactured. A material that has low autofluorescence properties may be desirable to minimize background contributions in the signal collection.

Because integrated lens 2410 is used in off-axis geometry, minor optical aberrations at the focus may be exhibited if the curved surface is circular. While a circular profile functionally works, the use of an aspheric surface may be employed to extend the range of the vertical position of the incident beam for which the beam will be coupled to waveguide 2405, allowing a larger range of adjustment of the angle β. The appropriate deviation from a circular profile can be calculated with optical ray tracing programs familiar to those skilled in the art.

The large area of the top surface of waveguide 2405 before the focus may allow for a sample chamber to be sealed. Gasket 2425 sealing surface may be absent from the optical path. Therefore, a larger range of gasket materials may be possible that only need to be evaluated for their chemical/biological compatibility and not their optical properties. For example, an adhesive backed spacer can be utilized to form a sealed flowcell without a complicated clamping mechanism. Multiple flow cells can also be incorporated into a single biosensor by utilizing a gasket with multiple channels.

FIG. 27 is a cavalier projection view illustrating an exemplary gasket 2705 with multiple channels. The width of each channel may be chosen to match the unfocused dimension of the incident beam such that light coupling to the gasket along the length of the waveguide is minimized. A mechanism for translating the incident beam between channels may be included. In addition, the top surface of waveguide 2405 within the flow channels may be appropriately treated to allow for the capture of fluorescently labeled target molecules such as proteins, RNA, DNA, or cellular structures.

A lid attached to the gasket completes the flow cell. Fluid samples can be introduced through orifices in the lid and flow through the channels, allowing the fluid to interact with the top waveguide surface. Fluid reservoirs exterior to the flow channel can also be included to allow the introduction of fluids into the flow channel and an overflow reservoir at the outlet port of the flow channel to contain the fluid after it has passed through the flow channel. With plastic components, the gasket may be optionally eliminated by molding the channels into one of the plastic components and joining the two plastic components directly with methods known to those skilled in the art (e.g., laser or ultrasonic welding).

The evanescent field created by the light within waveguide 2405 can excite fluorophores that have attached to the top surface of waveguide 2405. As the fluorophores relax and emit frequency shifted radiation, the emitted light may be captured by a lens or series of lenses (e.g., collection and filtering optics 2445) to transfer an image of the surface to a plane that is imaged by a light capturing device (e.g., imaging device 2450) such as a CCD or CMOS sensor. An optical filter may also be placed between the waveguide surface and the imaging device to eliminate scattered incident light that has not been frequency shifted by the captured fluorophores.

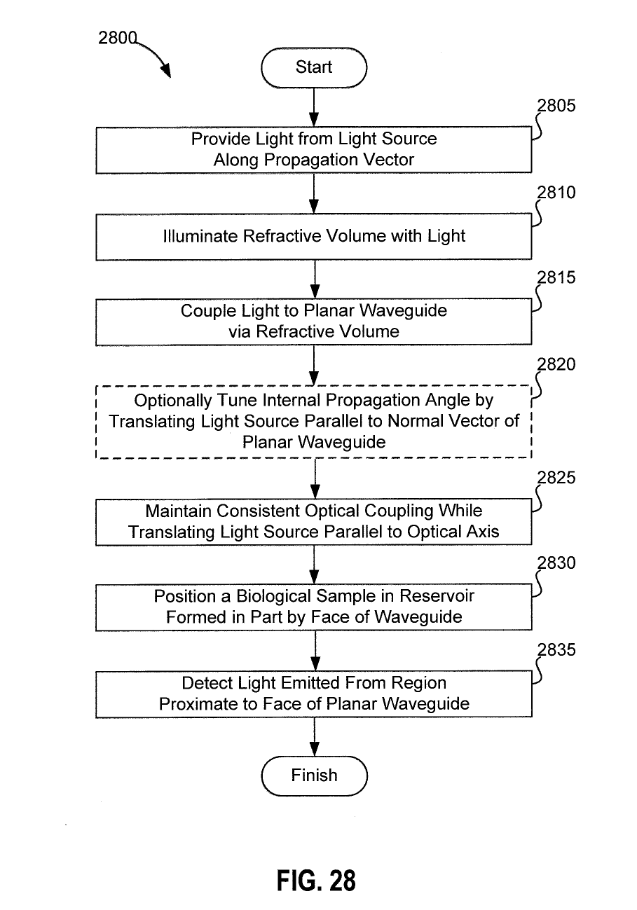

FIG. 28 is a flowchart of an exemplary method 2800 for performing sample analysis. The steps of exemplary method 2800 may be performed in varying orders. Furthermore, steps may be added or subtracted from exemplary method 2800 and still fall within the scope of the present technology. The methodology illustrated in FIG. 28 may be performed for fluorescence detection and assay based on evanescent fields.

In a step 2805, light is provided from a light source along a propagation vector. The light source may include a laser or any other source of collimated or near-collimated light.

In a step 2810, a refractive volume is illuminated with the light. The refractive volume is positioned proximate to, and may be integrated with, a planar waveguide. In exemplary embodiments, the refractive volume may include at least a section of a plano-convex cylindrical lens, wherein the longitudinal axis of the refractive volume is oriented perpendicular to the optical axis and the normal vector of the planar waveguide.

In a step 2815, the light is coupled to the planar waveguide via the refractive volume. The waveguide is oriented such that the propagation vector is perpendicular to the normal vector of the planar waveguide and offset from the planar waveguide in a direction parallel to the normal vector of the planar waveguide.

In an optional step 2820, indicated by a dashed box, the optical coupling of the light provided by the light source to the planar waveguide is tuned by translating the light source in a direction parallel to the normal vector of the planar waveguide.

In a step 2825, consistent optical coupling of the light provided by the light source to the planar waveguide is maintained while translating the light source parallel to the optical axis of the planar waveguide.

In a step 2830, a biological sample is positioned in a reservoir formed at least in part by a face of the planar waveguide.

In a step 2835, light emitted from a region proximate to a face of the planar waveguide is detected. In some embodiments, a detector is positioned to detect light emitted from a region proximate to the face of the planar waveguide having a plurality of capture molecules bound thereto.

For some applications, containment of the liquid layer within a sub-wavelength extent, as in the context of the applications described above, may be unfeasible. For instance, if the object of interest is a biological cell on the order of one to twenty microns in diameter, then a different approach to analyte illumination and light guiding is required.

Another important aspect to consider when designing optical waveguides for a practical application is the manufacturability of the waveguide, especially if the application is intended to enter volume production with cost requirements. The sensitivity to manufacturing tolerances must be evaluated as it can greatly influence the manufacturability and, in the worst case, render the design unfeasible. Likewise, the method for coupling light into the waveguide should be considered, since the light-insertion method may impact both the waveguide manufacturability and the engineering effort required to interface the waveguide with the light source. This issue is of particular concern if the light source will not be permanently affixed to the waveguide. Additionally, the interfacing complexity tends to increase as the waveguide dimensions decrease.

Although the coupling of light into micrometer-scale waveguides has been implemented in, for instance, telecommunications equipment, the engineering effort and manufacturing expenses are important factors to be considered for cost-sensitive applications outside of telecommunications. For instance, the various types of waveguides described above are generally inappropriate for mass production due to their complexity.

It would be desirable to use an optical waveguide to efficiently illuminate low-n media and/or objects embedded in such media, where the media or objects extend beyond the penetration depth of the evanescent field generated at a high-n to low-n interface. A low-n medium may be, for example, a material having an index of refraction lower than that of conventional solid materials, e.g., a refractive index less than ˜1.5. An optical waveguide capable of effectively illuminating a core containing a low-index of refraction medium is described herein. It is noted that the terms “light” and “illumination” are used interchangeably herein.

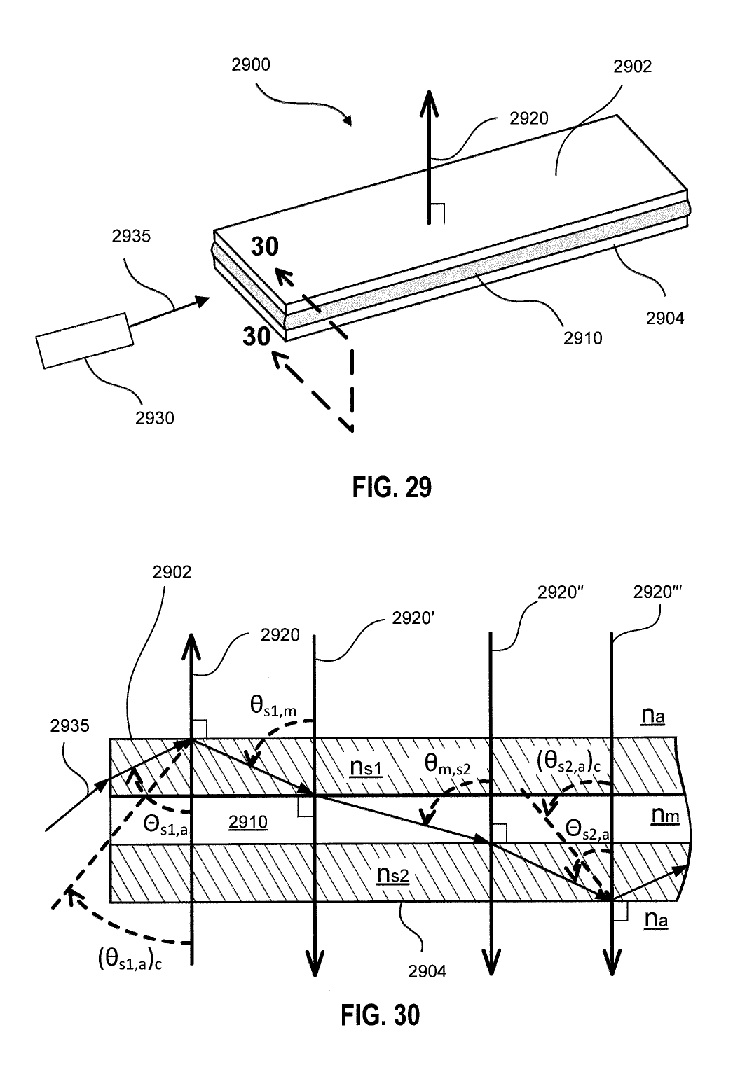

In an embodiment, as illustrated in FIG. 29, a planar waveguide 2900 includes a stack of layers formed from a first substrate 2902 and a second substrate 2904 sandwiching a low-n medium 2910. The low-n medium is interchangeably denoted herein as the interrogation medium. First and second substrates 2902 and 2904 may be, for instance, optically clear so as to be transparent to light having a wavelength within a predetermined range. Low-n medium 2910 is introduced between first and second substrates 2902 and 2904 such that first and second substrates 2902 and 2904 cooperate to confine low-n medium 2910 therebetween. First and second substrates 2902 and 2904 and low-n medium 2910 may have a variety of thicknesses, as long as low-n medium 2910 exhibits a lower refractive index in comparison to first and second substrates 2902 and 2904. The present concept is compatible with numerous schemes of coupling light into the waveguide, as well as different methods of containing the low-n medium therein. The low-n medium may be liquid, gaseous and/or solid.

One-dimensional optical confinement (i.e., in a direction indicated by a surface normal 2920, indicated by a thick arrow, of the first and second substrates) of light inserted into the waveguide may be provided by TIR at the interfaces between the optically clear substrates and the external surroundings. In the exemplary embodiment shown in FIG. 29, a light source 2930 directs illumination 2935 into planar waveguide 2900 at an angle away from the substrate normal and out of the plane of the substrates such that one-dimensional optical confinement of illumination 2935 is provided by planar waveguide 2900 by total internal reflection at the two substrate-to-surrounding medium interfaces.

A cross-sectional view of planar waveguide 2900 is shown in FIG. 30. It should be noted that the figures are not drawn to scale. As shown in FIG. 30, first substrate 2902 has a refractive index ns1, second substrate 2904 has a refractive index ns2, and low-n medium 2910 has a refractive index nm. Planar waveguide 2900 is surrounded by air (or some other medium) with a refractive index na. The indices of refraction fulfill the requirements:

nₐ<nₛ₁, nₛ₂ [Eq. 4] and

nₐ<nₘ. [Eq. 5]

Note that critical angle for (θ_{—}1,2)_c for light propagation from a first material (with refractive index n1) toward a second material (with refractive index n2, where n2<n1) is given by:

$${( {θ}_{1 , 2} )}_{c} = arcsin ( \frac{{n}_{2}}{{n}_{1}} ) \qquad [ Eq . 6 ]$$

As shown in FIG. 30, light 2935 enters planar waveguide 2900 such that an incidence angle θ_(s-a) from first substrate 2902 (with refractive index ns1) into the surrounding medium (with refractive index na) is greater than the critical angle (θ_(s,a))_c as defined from the lower of ns1 and ns2, i.e.,

θ_{s,a}>(θ_{s,a})_{c}, [Eq. 7]

such that light 2935 is contained within planar waveguide 2900 by TIR. All angles are measured relative to surface normal 2920. Consequently, the substrates and the interrogation medium form a multi-part waveguide, together providing light confinement in one dimension (i.e., in a direction parallel to surface normal 2920). The interrogation medium can be of any type (e.g., gaseous, liquid, and biological objects embedded in a liquid) as long as the refractive index condition of Eq. 4 and incidence angle condition of Eq. 7 are satisfied.

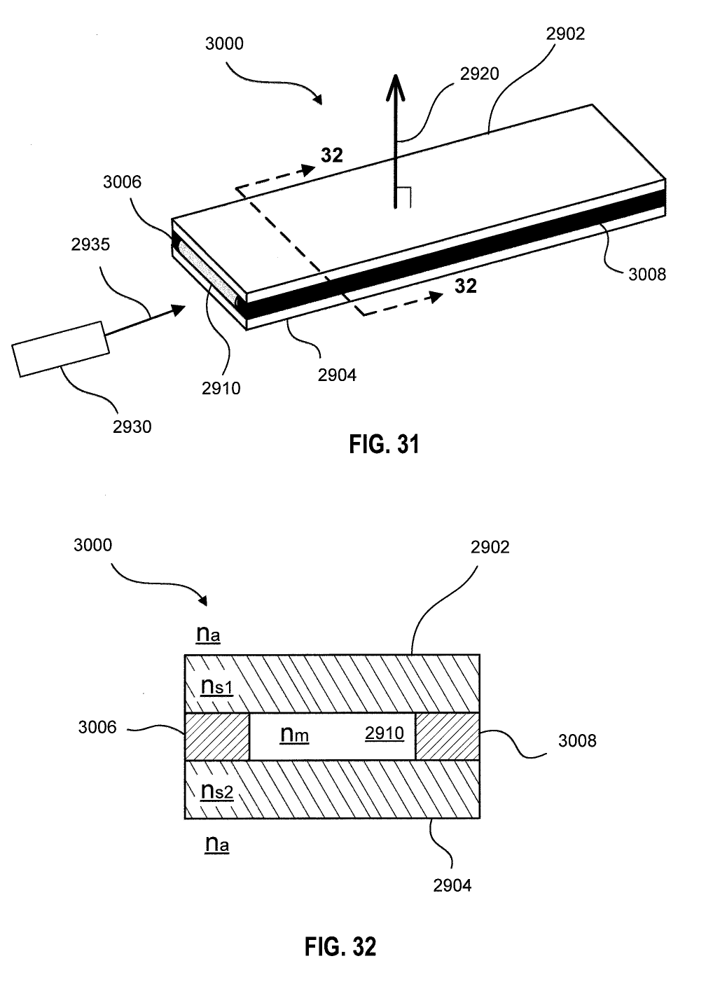

For liquid and gaseous interrogation media, the waveguide design may be modified for containing the interrogation medium. For example, in the embodiment shown in FIGS. 29 and 30, low-n medium is contained between first and second substrates 2902 and 2904 entirely by surface tension. FIGS. 31 and 32 show an alternative configuration for a planar waveguide 3000, in which first and second substrates 2902 and 2904 are spaced apart by first and second gaskets 3006 and 3008. Still alternatively, first and second gaskets 3006 and 3008 may be connected to form a single contiguous gasket. It is noted that the embodiments shown in FIGS. 29-32 accommodates the addition of inlet and outlet ports (not shown) for the low-n, interrogation medium. The open ends in FIGS. 31 and 32 may be plugged using another material, thereby forming a completely-sealed volume for containing the interrogation medium.

The containment configuration should be compatible with the method for coupling light into the waveguide. For instance, the system may be configured such that the interrogation medium may be uniformly illuminated in the plane of the planar waveguide, even if the light is not solely confined within the interrogation medium. In-coupling of light 2935 through the substrates is generally unaffected by the low-n medium containment schemes shown in FIGS. 29-32. Interference effects or curved interface effects (e.g., if light 2935 is incident from the surrounding medium directly onto low-n medium 2910, which may include an interface curvature caused by surface tension) may affect subsequent propagation of light 2935 through planar waveguide 2900 or 3000.

Referring to FIG. 30, the illumination strength inside low-n medium 2910 depends on the angle of light propagation inside planar waveguide 2900. Due to spatial compression, light propagation at angles close to the critical angle will result in greater illumination strength than light propagating at angles far from the critical angle. To a first approximation, the average illumination strength within planar waveguide 2900 is inversely proportional to sin(θs,a), where θs,a is the incidence angle of propagating light at the substrate-air interface such that the light is contained within the waveguide.

Referring to FIGS. 29 and 30, the manner of coupling light into the waveguide may be chosen in accordance with the given application. For example, the incident light may be coupled into a single layer of the multi-part, planar waveguide, any combination of layers, or all layers. If the light is coupled directly into the low-n interrogation medium, for instance, the light may be inserted into the planar waveguide at any angle such that Eq. 7 is fulfilled. This range of angles include normal incidence onto the waveguide end (i.e., at an angle perpendicular to surface normal 2920). On the other hand, if the light is coupled in through one of the substrates, the angle of incidence should further satisfy the conditions:

θ_{s1,m}<(θ_{s1,m})_{c} [Eq. 8] and

θ_{s2,m}<(θ_{s2,m})_{c} [Eq. 9]

at the interfaces from first or second substrate 2902 and 2904 into low-n medium 2910, where the subscript c denotes critical angle. Fulfillment of the appropriate one of these conditions ensures that light is eventually coupled from the substrate into the low-n medium.

A simple version of the planar low-n index waveguide may be formed from two identical substrates of a single type of material as shown in FIG. 30. Alternatively, the two substrates may be non-identical and even be composed of several disparate layers of optically-clear materials, possibly with different indices of refraction.

Note that, if first or second substrate 2902 or 2904 is formed of a plurality of disparate layers, the effective refractive index of the combination of the plurality of disparate layers may be expressed as n_{eff}, which is related to the refractive index nₐ of the surrounding medium by the equation:

nₐ<n_{eff} [Eq. 10]

Furthermore, the two substrates may be in contact with different media, such as if first substrate 2902 is exposed to air while second substrate 2904 is attached to a third substrate (not shown). In this case, multi-part planar waveguide 2900 will still work as a waveguide as long as Eqs. 1 and 4 and the additional condition:

nₐ<nₘ, n_{eff} [Eq. 11]

are satisfied for both substrates and surrounding media.

The angle of light propagation should be such that the incidence angle θ for the substrate-to-interrogation medium interface, as well as all interfaces between layers forming the substrate, satisfy the condition:

θ<θ_{c} [Eq. 12]

and, for interfaces at the substrate and the surrounding medium, the incidence angle θ from the substrate to the surrounding medium should fulfill the condition:

θ>θ_{c} [Eq. 13]

The embodiments illustrated in FIGS. 29-32 impose no constraints on the thicknesses of the interrogation medium or the two substrates as long as the refractive index and incidence angle requirements of Eqs. 1 and 4 are fulfilled. The disclosed embodiments may be particularly suitable for low-cost, volume production and may be combined with light coupling mechanisms of relatively low complexity. While planar waveguides 2900 and 3000 will function properly with virtually any choice of thicknesses of the interrogation medium and substrates, the actual choice of layer thicknesses may be based on a number of factors, such as the choices of materials, manufacturing methods and cost.

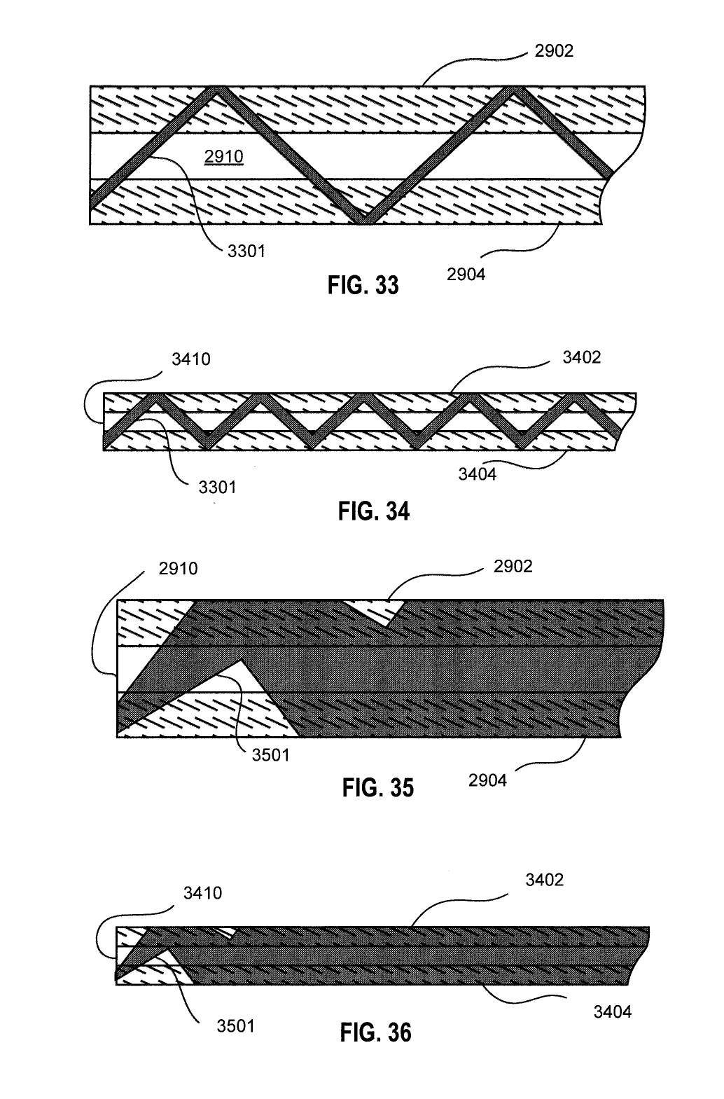

The light propagation through thick and thin versions of planar waveguide 2900 is illustrated for both a collimated beam (FIGS. 33 and 34) and a diverging beam (FIGS. 35 and 36) as the light input. As shown in FIGS. 33 and 34, a collimated beam 3301 will make distinct passes through low-n medium throughout the waveguide with high intensity. For a diverging beam 3501, on the other hand, the reflected light eventually overlaps, resulting in substantially uniform illumination within the planar waveguide. Consequently, if only one or more, appropriately-placed small regions, extending no more than the portion illuminated by a single pass, require illumination, then collimated beam 3301 can provide greater intensity than diverging beam 3501 within the small region. If the intent is to illuminate a larger region, possibly in a uniform fashion, then a diverging beam 3501 may be a better choice. It should also be noted that the pairs of figures (i.e., FIGS. 33-34 and FIGS. 35-36) may be viewed as illustrations of the same planar waveguide but illuminated with collimated and diverging beams, respectively, of different beam diameters.

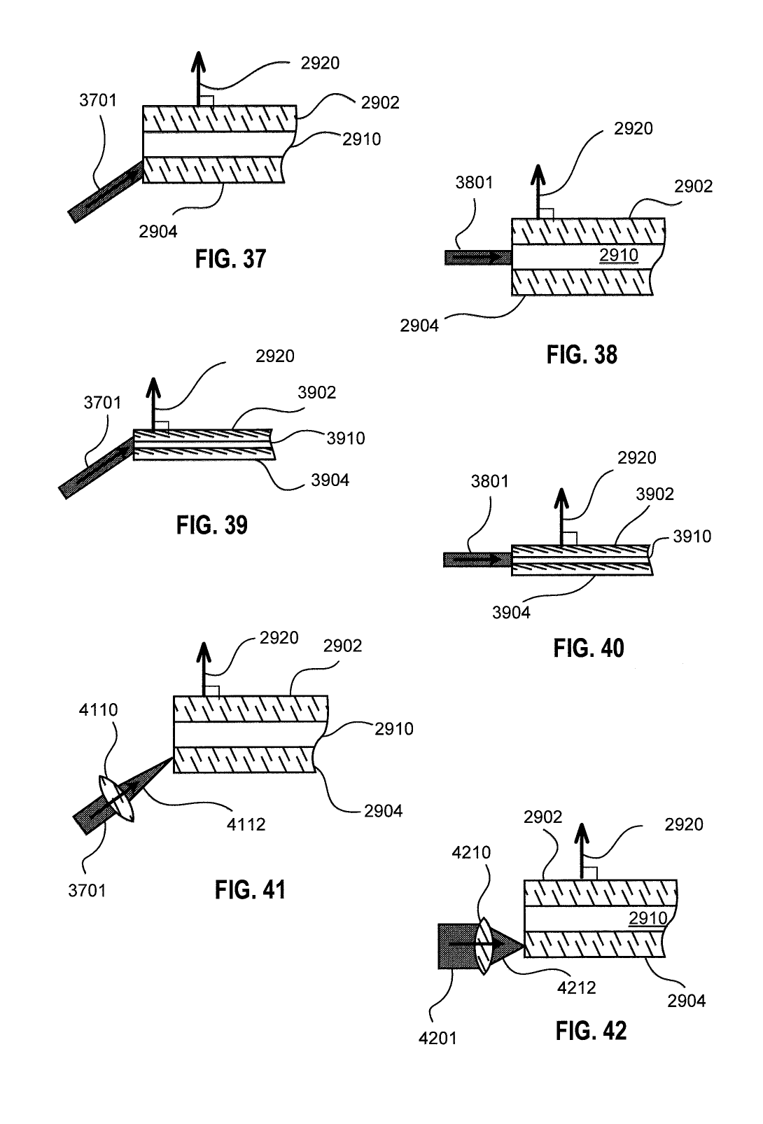

Efficient coupling of light into the waveguide is readily achieved with a combined waveguide thickness of macroscopic extent, e.g., on the order of few hundreds of nanometers or greater. For instance, a focused laser beam may be easily coupled into a planar waveguide of such dimensions. The mechanism for appropriately focusing the incoming light may be either integrated in the waveguide or constructed as a system separate from the waveguide. Examples of light coupling mechanisms are shown in FIGS. 37-46.

FIG. 37 shows an embodiment, in which a light beam 3701 is incident at an angle away from surface normal 2920 onto second substrate 2904. FIG. 38 shows a special case, in which a light beam 3801 is directly incident on low-n medium 2910 at an angle perpendicular to surface normal 2920. FIG. 39 shows a thin, planar waveguide embodiment, in which light beam 3701 is simultaneously incident on first and second thin substrates 3902 and 3904, respectively, and low-n medium 3910, again at an angle away from surface normal 2920. FIG. 40 again shows the thin, planar waveguide formed from first and second thin substrates 3902 and 3904, respectively, and low-n medium 3910, with light beam 3801 being inserted into all three layers at an angle perpendicular to surface normal 2920.

FIGS. 41 and 42 show embodiments in which an external lens is used to focus the incident light beam onto one of the two substrates. FIG. 41 shows an embodiment, in which a lens 4110 is used to focus light beam 3701 such that a focused beam 4112, which is incident from a non-normal angle away from surface normal 2920, is directed into second substrate 2904. Similarly, FIG. 42 shows an embodiment, in which a light beam 4201, incident at an angle perpendicular to surface normal 2920, is focused by a lens 4210 to form a focused beam 4212 before being incident on second substrate 2904.

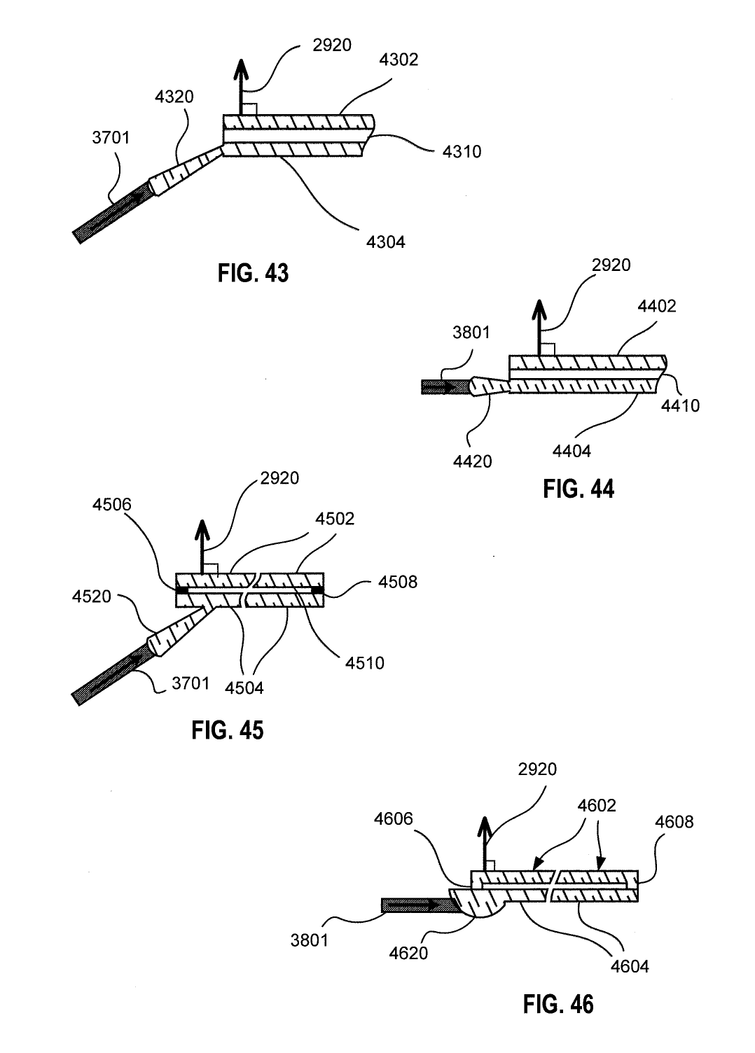

In another approach, the light may be coupled into one of the two substrates, which is equipped with an integrated lens assembly for appropriately focusing and directing the incoming light. For instance, FIG. 43 shows an embodiment, in which first and second substrates 4302 and 4304, respectively, are spaced apart to contain a low-n medium 4310 therebetween. Second substrate 4304 includes an integrated lens 4320, which is configured to receive light beam 3701 so as to couple light beam 3701 into second substrate 4304 and, subsequently, the multi-part planar waveguide configuration. FIG. 44 shows a similar embodiment, in which first and second substrates 4402 and 4404, respectively, is spaced apart to contain a low-n medium 4410 therebetween. In this embodiment, second substrate 4404 includes an integrated lens 4420, which is this time configured to receive light beam 3801, incident at an angle perpendicular to surface normal 2920. Light beam 3801, received at integrated lens 4420, is directed into second substrate 4404 and, subsequently, the multi-part planar waveguide as a whole. FIG. 45 shows an alternative embodiment, which includes first and second substrates 4502 and 4504, respectively, separated by first and second gaskets 4506 and 4508, respectively, so as to contain a low-n medium 4510 therebetween. Second substrate 4504 includes an integrated lens 4520, which is configured to receive light beam 3701 at a portion of second substrate 4504 away from first gasket 4506 such that light beam 3701 is inserted into the multi-part planar waveguide structure without being blocked by first gasket 4506. Finally, FIG. 46 shows an embodiment including first and second substrates 4602 and 4604, respectively. This time, rather than including a separate gasket, first substrate 4602 includes first and second stand-offs 4606 and 4608, respectively, which are configured so as to be attachable to second substrate 4604 by, for instance, laser welding, ultrasonic welding, or other suitable bonding method. When bonded together, first and second substrates 4602 and 4604, respectively, defines a volume for containing a low-n medium 4610 therebetween. Second substrate 4604 includes an integrated lens 4620 configured for receiving light beam 3801, incident at an angle perpendicular to surface normal 2920, such that light beam 3801 propagates into second substrate 4604 and, subsequently, into the multi-part planar waveguide structure as a whole. Integrated lens 4620 may be, for example, an integrated lens as described in the aforementioned U.S. patent application Ser. No. 12/617,535, such that insertion of light beam 3801 into second substrate 4604 is substantially insensitive to translation of light beam 3801 with respect to integrated lens 4620.

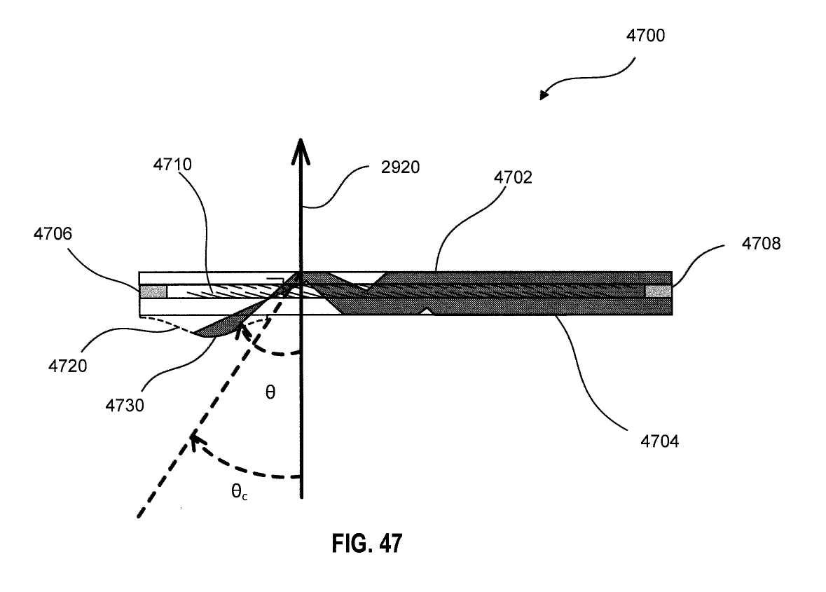

FIG. 47 shows a side view of an exemplary waveguide structure, shown here to illustrate insertion, propagation and containment of a light beam therethrough. A planar waveguide 4700 includes first and second substrates 4702 and 4704, respectively, spaced apart by first and second gaskets 4706 and 4708, respectively, so as to contain a low-n medium 4710 therein. Second substrate 4704 may optionally include a refractive component, such as an integrated lens 4720 (shown as a dashed curve), for facilitating insertion of a light beam 4730 into planar waveguide 4700. As shown in FIG. 47, first and second substrates 4702 and 4704, respectively, low-n medium 4710, and incident angle θ fulfill the refractive index and incident angle conditions specified in Eqs. 1 and 4 above such that, after a few TIR bounces at the substrate-air interfaces, light beam 4730 uniformly illuminates the thickness of planar waveguide 4700.

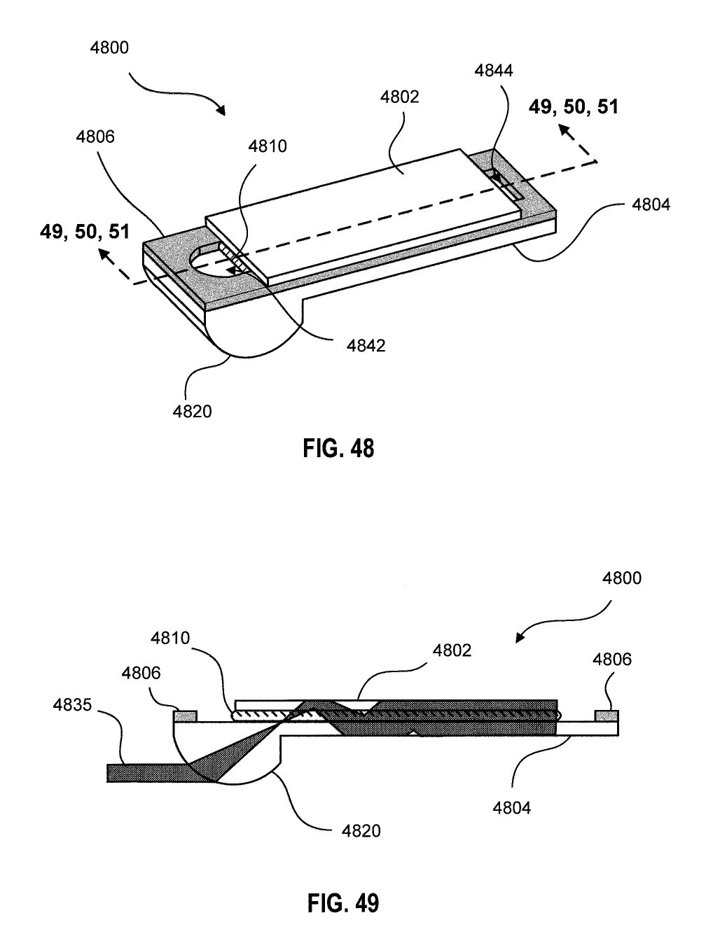

An exemplary embodiment of a cartridge system with interrogation medium containment, in- and outlet ports, and light-coupling means designed for light entry into the waveguide inside the contained region is shown in FIGS. 48-49. A waveguide cartridge 4800 includes first and second substrates 4802 and 4804, respectively, separated by a gasket 4806 so as to provide containment of a low-n medium 4810 therebetween. Second substrate 4804 includes an integrated lens 4820 for receiving light 4835 incident thereon and directing light 4835 into waveguide cartridge 4800 so that, after a few TIR bounces therein, light 4835 uniformly illuminates at least a portion of low-n medium 4810. Waveguide cartridge 4800 further includes an inlet port 4842 and an outlet port 4844, through which one or more samples may be introduced into waveguide cartridge 4800 as low-n medium 4810.

The use of optically-clear substrates may facilitate optical communication with the interrogation medium through the substrates. For instance, additional image capture through the substrates may be utilized to detect light emitted from the interrogation medium and thereby extracting information about the interrogation medium in, e.g., microscopy and/or fluorescence applications. Additionally, by using a position-sensitive detector, spatial information regarding the interrogation medium may be obtained. Alternatively, light emitted within the range of angles confined by the waveguide may be detected in the plane of the waveguide, if an appropriate pathway is established for allowing this light to exit the waveguide (not shown). For example, a mechanism for out-coupling of light may be incorporated into the substrate in a manner similar to that used for the in-coupling of light.

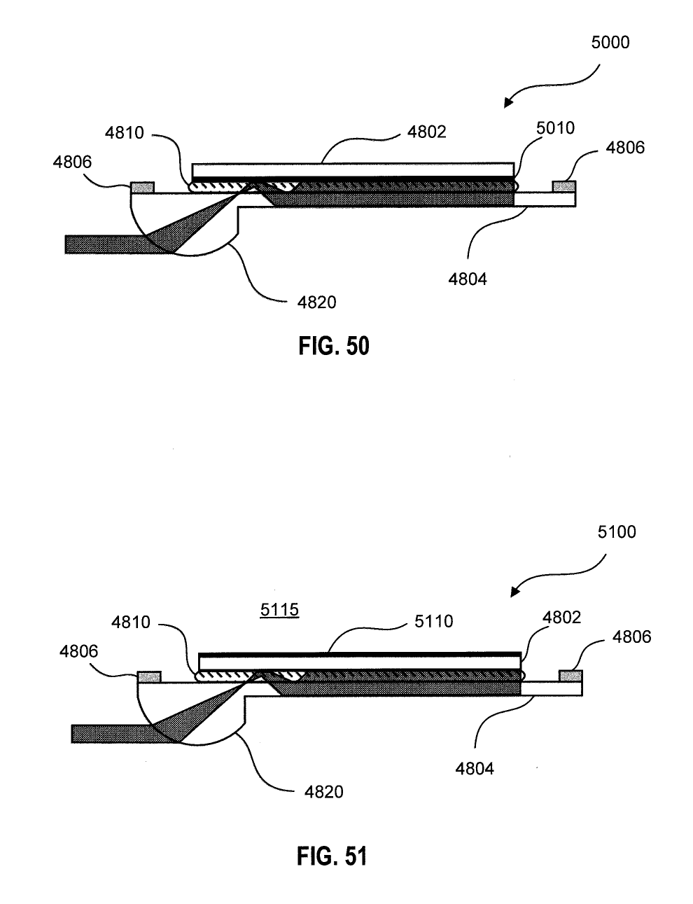

As an alternative, one or more of the substrate-surrounding medium interfaces may be configured to be at least partially reflective. Additionally, one or more reflecting surfaces may be utilized in the waveguide. For instance, one or both of the substrate-to-interrogation medium interfaces may be configured to be partially or completely reflective in order to better contain the guided light within the interrogation medium. In the case of configurations wherein the light is coupled into the waveguide through one of the two substrates, the other one of the two substrates may be configured to include a reflective surface (e.g., at the substrate-to-interrogation medium interface), thereby increasing the illumination intensity within the interrogation medium. An example of this configuration is shown in FIG. 50, in which a waveguide cartridge 5000 further includes a reflective layer 5010 at the interface between first substrate 4802 and low-n medium 4810. The configuration as shown in FIG. 50 still allows for optical communication through second substrate 4804 (e.g., for detection of light emitted from the interrogation medium), while improving the light containment within waveguide cartridge 5000 without affecting the in-coupling of light therein. Another advantage of this configuration is a reduced distance from light entry to uniform illumination, when guiding a diverging beam. Still another example is shown in FIG. 44, in which a waveguide cartridge 4400 includes a reflective layer 4410 at the interface between the outer surface of first substrate 4802 and surrounding medium 4415. The advantages imparted in the configuration of FIG. 44 is similar to those discussed in relation to FIG. 50.

Other variations, in which one or both of the substrates include one or more reflective regions, may hold other advantages. For instance, the configuration depicted in FIG. 49 may be modified to include a reflective section located at a certain distance from the point of light entry, thereby reducing the distance required to achieve uniform illumination while maintaining means for optical communication through both substrates. Additionally, the at least partially reflective surfaces in FIGS. 50 and 51 may be used to direct light emitted by the interrogation medium (e.g., fluorescence emission) towards a detector placed underneath waveguide 100.

While each of the illustrated embodiments shows a single light beam entering the waveguide, the embodiments may be extended to accommodate multiple beams entering the waveguide. For example, the waveguide may be constructed to accept multiple beams of light by in-coupling several light beams through one port, such as a lens integrated into one of the substrates, and/or by incorporating several in-coupling ports. The beams may propagate in directions that are parallel to each other, either in co- or counter-propagating configurations, or in non-parallel configurations.

### EXAMPLE 7

### Detection of Fluorescently Labeled Human Blood Cells

Human peripheral blood mononucleocytes (“PBMCs”) are labeled with CD3 Alexa Fluor 647 fluorescence stain, available from Invitrogen Corporation. The cells, whose diameter is 6-12 μm, are kept in a buffer consisting of phosphate buffered saline with 1% Bovine Serum Albumin and 0.06% sodium azide. The buffer with cells is loaded into a cartridge of the type shown in FIGS. 48 and 49. The substrate materials and the buffer lead to a critical angle at the substrate-to-interrogation medium interface of θc=61°. 635 nm laser light is coupled into the system through the curved part of the lower substrate. The curvature is designed such that different entry heights result in different angles of incidence onto the substrate-to-interrogation medium interface. Two different laser heights were used in the present example resulting in two different angles of incidence onto the substrate-to-interrogation medium interface: (a) 57° and (b) 66°. With a laser divergence angle of 3.5° after passing through the curved surface of the lower substrate case (a) allows the light to pass through the interrogation medium and be guided by the entire cartridge as shown in FIG. 49. In case (b), on the other hand, the laser light is confined to the lower substrate and the interrogation medium is illuminated only by the evanescent field. The 635 nm laser light excites the Alexa Fluor 647 fluorophores and an imaging system positioned underneath the cartridge images fluorescence emitted from the interrogation medium.

TABLE 1

| | Case (a) | Case (b) |
|---|---|---|
| | Using the low-n core | Evanescent |
| | waveguide configuration | illumination |
| | θ = 57° | θ = 66° |
| # cells detected | 590 | 138 |
| Staining percentage | 56% | 13% |
| S/N for representative cell | 4.4 | 1.2 |

Raw fluorescence images (not shown) indicate that the fluorescence is strongly enhanced when the interrogation medium is directly illuminated, i.e., case (a). The results are summarized in TABLE 1. In case (a), 590 fluorescent cells are detected versus only 138 cells in case (b). The staining percentage, i.e., number of fluorescent cells divided by total number of cells, for case (a) agrees with results obtained on a flow cytometer. The signal to noise ratio, S/N, has been calculated as the peak pixel intensity of a representative cell divided by the standard deviation of the surrounding background pixel intensities. Alternatively, the signal to noise ratio could have been calculated as the peak intensity of a cell divided by the background level. However, the former method is the more appropriate parameter when concerned with the ability to distinguish a cell from the background in the images. As listed in TABLE 1, the signal to noise ratio increases almost fourfold when directly illuminating the interrogation medium.

The following list of items describes various embodiments of the present disclosure.

Item 1. An apparatus for illuminating a sample, the apparatus including:
a waveguide including
a first substrate including a first outer surface and a first inner surface, and a second substrate including a second outer surface and a second inner surface, the first and second inner surfaces of the first and second substrates, respectively, being spaced apart from each other and partly defining a volume for confining the sample therein; and
a light source for providing light directed toward the waveguide such that the light is optically coupled to and contained within the waveguide between the outer surfaces of the first and second substrates, while illuminating at least a portion of the sample confined within the volume.

Item 2. The apparatus of item 1, the sample containing at least one object, wherein the waveguide and the light source are configured to cooperate to uniformly illuminate the at least one object.

Item 3. The apparatus of item 2, where in the at least one object is greater than one micrometer in diameter.

Item 4. The apparatus of item 1, further including a gasket for separating the first and second inner surfaces of the first and second substrates, respectively, while further defining the volume for confining the sample therein.

Item 5. The apparatus of item 1, wherein the light is contained between the outer surfaces of the first and second substrates at least in part by total internal reflection.

Item 6. The apparatus of item 1, wherein the light source provides collimated light.

Item 7. The apparatus of item 6, further including a refractive element for diverging the collimated light within the planar waveguide.

Item 8. The apparatus of item 1, wherein the first and second surfaces are spaced apart by a distance of more than 10 microns.

Item 9. The apparatus of item 8, wherein the first and second surfaces are spaced apart by a distance on an order of 100 microns.

Item 10. The apparatus of item 1, wherein at least one of the first and second outer surfaces and first and second inner surfaces is configured for at least partially reflecting light incident thereon.

Item 11. The apparatus of item 10, wherein the at least one of the first and second outer surfaces and first and second inner surfaces is further configured for reflecting light of a predetermined wavelength range incident thereon.

Item 12. The apparatus of item 1, further including a refractive assembly for optically coupling the light from the light source into the second substrate.

Item 13. The apparatus of item 12, wherein the second substrate and the refractive assembly are integrally formed from a single piece of material.

Item 14. The apparatus of item 13, wherein the second substrate and the refractive assembly are formed by injection molding.

Item 15. The apparatus of item 12, wherein the refractive assembly is configured such that a relative translation of the light source in a plane parallel to the inner surface of the second substrate is inconsequential to optical coupling of the light from the light source to the waveguide.

Item 16. The apparatus of item 12, a surface normal being defined as a vector perpendicular to the outer surface of the second substrate, wherein the light is incident on the refractive volume at a non-90° angle away from the surface normal.

Item 17. A sample analysis system including:
a waveguide including
a first substrate including a first outer surface and a first inner surface, and a second substrate including a second outer surface and a second inner surface, the first and second inner surfaces of the first and second substrates, respectively, being spaced apart from each other and partly defining a volume for confining a sample therein; and
a first light source for providing a first illumination directed toward the waveguide such that the first illumination is optically coupled to and contained within the waveguide between the outer surfaces of the first and second substrates while illuminating at least a portion of the sample confined within the volume; and
a detector for detecting a first light signal emitted from the sample as a result of the first illumination interacting with the portion of the sample.

Item 18. The system of item 17, the sample containing at least one object, wherein the waveguide and the light source are configured to cooperate to uniformly illuminate the at least one object.

Item 19. The system of item 18, wherein the at least one object is greater than one micrometer in diameter.

Item 20. The system of item 17, further including:
a second light source configured for providing a second illumination; and
imaging optics for directing the second illumination from the second light source to at least another portion of the sample and to the detector,
wherein the detector is further configured for detecting a second light signal resulting from the second illumination interacting with the at least another portion of the sample.

Item 21. The system of item 17, further including a gasket for separating the first and second inner surfaces of the first and second substrates, respectively, while further defining the volume for confining the sample therein.

Item 22. The system of item 17, wherein the light is contained between the outer surfaces of the first and second substrates at least in part by total internal reflection.

Item 23. The system of item 17, wherein the light source provides uncollimated light.

Item 24. The system of item 17, wherein at least one of the first and second outer surfaces and first and second inner surfaces is configured for at least partially reflecting light incident thereon.

Item 25. The system of item 24, wherein the at least one of the first and second outer surfaces and first and second inner surfaces is further configured for reflecting light of a predetermined wavelength range incident thereon.

Item 26. The system of item 17, further including a refractive assembly for optically coupling the light from the light source into the second substrate.

Item 27. The system of item 26, wherein the second substrate and the refractive assembly are integrally formed from a single piece of material.

Item 28. The system of item 27, wherein the second substrate and the refractive assembly are formed by injection molding.

Item 29. The system of item 26, wherein the refractive assembly is configured such that a relative translation of the light source in a plane parallel to the inner surface of the second substrate is inconsequential to optical coupling of the light from the light source to the planar waveguide.

Item 30. The system of item 26, a surface normal being defined as a vector perpendicular to the outer surface of the second substrate, wherein the light is incident on the refractive volume at an angle away from the surface normal.

Item 31. A system for analyzing a sample containing one or more target analytes, said system comprising a cartridge and a reader instrument,
said cartridge comprising a first substrate and a second substrate,
   said first substrate comprising a planar waveguide, said planar waveguide having a first outer surface and a first inner surface,
   said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample;
said reader instrument comprising
   a receiving mechanism for positioning the cartridge therein, and
   an imaging detector for detecting light signal from a field of view on or near the first inner surface of said planar waveguide;
   said cartridge further comprising a refractive volume for optically coupling a light beam provided by a light source to the planar waveguide, wherein said refractive volume is configured for refracting the light beam such that the light beam is focused on the first inner surface of the planar waveguide at a non-zero, internal propagation angle relative to the first inner surface for all light within the light beam.

Item 32. The system of Item 31, wherein said reader instrument further comprises a module for analyzing the light signal received by the imaging detector.

Item 33. The system of Item 31, wherein said distance between said first inner surface and said second inner surface, together with said field of view having measurable dimensions defines a sample volume.

Item 34. The system of Item 31, wherein said refractive volume is integrally formed from the planar waveguide.

Item 35. The system of Item 31, wherein said first inner surface is modified to create an attachment surface, said modification enhancing immobilization of said target analytes onto said first inner surface.

Item 36. The system of Item 35, wherein said modification is performed by using a cationic polymer.

Item 37. The system of Item 36, wherein said modification is performed by using a poly-lysine.

Item 38. The system of Item 35, wherein said modification is performed by immobilizing adhesive proteins on said first inner surface.

Item 39. The system of Item 35, wherein said modification is performed by immobilizing antibodies on said first inner surface.

Item 40. The system of Item 39, wherein said antibodies are selected from the group consisting of anti-CD3, anti-CD4, anti-CD8, anti-CD14, anti-CD45, and anti-TCR-beta antibodies.

Item 41. The system of Item 35, wherein said modification is performed by depositing an organosilane thin film.

Item 42. The system of Item 31, wherein said organosilane is an amino-silane.

Item 43. The system of Item 31, wherein said planar waveguide is made of plastic.

Item 44, The system of Item 43, wherein said plastic is a low-autofluorescence plastic.

Item 45. The system of Item 43, wherein said plastic is a cyclic olefin.

Item 46. The system of Item 43, wherein said plastic is a cyclic olefin polymer.

Item 47. The system of Item 31, wherein said refractive volume is a lens.

Item 48. The system of Item 34, wherein said refractive volume is a lens.

Item 49. The system of Item 34, wherein said refractive volume and said planar waveguide are made of plastic.

Item 50. The system of Item 34, wherein refractive volume and said planar waveguide are integrally formed by plastic injection molding.

Item 51. The system of Item 31, wherein said target analytes are labeled with one or more excitable tags.

Item 52. The system of Item 31, wherein said target analytes are labeled with antibodies conjugated with fluorophore.

Item 53. The system of Item 32, wherein said analyzing by said module comprises enumerating said target analytes by counting the number of target analytes in at least one field of view captured by said imaging detector of said reader instrument.

Item 54. The system of Item 53, wherein said number of target analytes in said at least one field of view represent total number of all target analytes in said sample volume, and concentration of said target analytes in said sample may be calculated by dividing the total number of target analytes obtained in Item 53 by the sample volume.

Item 55. The system of Item 54, wherein said concentration is insensitive to the amount of said sample added to said sample chamber.

Item 56. The system of claim 31, wherein the reader instrument further comprises one or more light sources for providing the light beam.

Item 57. The system of claim 56, wherein the one or more light sources are lasers.

Item 58. The system of Item 31, wherein at least a fraction of the light within said light beam is optically coupled to and contained between the first outer surface of the first substrate and the second outer surface of the second substrate, wherein said fraction of the light illuminates at least a portion of the sample confined within the sample chamber.

Item 59. The system of Item 31, wherein said reader instrument further comprises an autofocus means.

Item 60. The system of Item 31, wherein said reader instrument further comprises a light source and a condenser lens, said light source and condenser lens, in conjunction with said imaging detector, are capable of generating a brightfield microscopy image.

Item 61. The system of Item 31, wherein said reader instrument comprises an actuation means for generating multiple fields of view.

Item 62. The system of Item 31, wherein said reader instrument comprises an actuation means for generating multiple fields of by controlling relative position of said cartridge and said imaging detector.

Item 63. The system of Item 31, wherein said reader instrument comprises an actuation means for generating multiple fields of view by moving said cartridge relative to said imaging detector.

Item 64. The system of Item 31, wherein said reader instrument comprises an actuation means for generating multiple fields of view by moving said imaging detector relative to said cartridge.

Item 65. A method for analyzing a sample containing one or more target analytes, the method comprising:
(a) contacting said sample with a labeling molecule of a first type
(b) introducing said sample prepared in step (a) into a cartridge, said cartridge comprising a first substrate, a second substrate, and a refractive volume for optically coupling light to the planar waveguide,
   said first substrate comprising a planar waveguide, said planar waveguide having a first outer surface and a first inner surface,
   said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample,
(c) positioning said cartridge in a reader instrument, wherein said instrument directs a light beam from a light source into said refractive volume such that the light beam is focused on the first inner surface of the planar waveguide at a non-zero, internal propagation angle relative to the first inner surface for all light within the light beam, wherein said light beam excites said labeling molecule of a first type bound to said one or more target analytes at said first inner surface of said planar waveguide;
(d) generating an image of a field of view having measurable dimensions at the first inner surface of said planar waveguide, said image resolving target analytes present in said sample.

Item 66. A method for analyzing a sample containing one or more target analytes, the method comprising:
(a) contacting said sample with labeling molecules of a first type and labeling molecules of a second type;
(b) introducing said sample prepared in step (a) into a cartridge, said cartridge comprising a first substrate, a second substrate, and a refractive volume for optically coupling light to the planar waveguide,
   said first substrate comprising a planar waveguide, said planar waveguide having a first outer surface and a first inner surface,
   said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample,
(c) positioning said cartridge in a reader instrument, wherein said instrument directs a first light beam from a first light source into said refractive volume, wherein said first light beam excites said labeling molecules of the first type, and wherein said instrument further directs a second light beam from a second light source into said refractive volume wherein said second light beam excites said labeling molecules of the second type;
(d) generating a first image and a second image of a single field of view having measurable dimensions at the first inner surface of said planar waveguide, wherein said images are spatially registered, and
(e) resolving target analytes present in the sample using said spatially registered images to generate differential labeling information.

Item 67. The method of Item 66, wherein said first light beam and said second light beam are focused on the first inner surface of the planar waveguide at a non-zero, internal propagation angle relative to the planar surface for all light within the light beams.

Item 68. The method of Item 66, wherein said target analytes in a field of view image represent all target analytes in the known sample volume defined by the said known distance between said first inner surface and said second inner surface, and the said field of view with known dimensions, thereby generating a concentration result in the form of number of target analytes per unit volume.

Item 69. The method of Item 66, wherein the target analytes include CD4 helper T cells.

Item 70. The method of Item 66, wherein said analyzing further comprises an algorithm for comparing said spatially registered images and for identifying specific target analytes using said differential staining information.

Item 71. The method of Item 70, wherein the target analytes include CD4 helper T cells, and wherein said differential staining information comprises anti-CD4 label information in a first spatially registered image, and anti-CD14 label information in a second spatially registered image, said analyzing further comprising identifying target analytes that are present in the anti-CD4 label image but not present in the anti-CD14 label image, and further reporting the anti-CD4 positive/anti-CD14 negative target analytes as CD4 helper T cells.

Item 72. The method of Item 70, wherein the target analytes include CD4 helper T cells, and wherein said differential staining information comprises anti-CD4 label information in a first spatially registered image, and anti-CD3 label information in a second spatially registered image, said analyzing further comprising identifying target analytes that are present in the anti-CD4 label image and also present in the anti-CD3 label image, and further reporting the anti-CD4 positive/anti-CD3 positive target analytes as CD4 helper T cells.

Item 73. The method of Item 66, further comprising generating a third spatially registered image of said field of view, wherein the third spatially registered image is a brightfield microscopy image.

Item 74. The method of Item 73, wherein the target analytes include CD4 helper T cells, and
wherein said brightfield microscopy image is used to discriminate non-target analyte objects in the field of view,
wherein said differential staining information comprises anti-CD4 label information in a first spatially registered image, and anti-CD14 label information in a second spatially registered image, said analyzing further comprising identifying target analytes that are present in the anti-CD4 label image but not present in the anti-CD14 label image, and further reporting the anti-CD4 positive/anti-CD14 negative target analytes as CD4 helper T cells.

Item 75. The method of Item 73, wherein the target analytes include CD4 helper T cells, and
wherein said brightfield microscopy image is used to discriminate non-target analyte objects in the field of view,
wherein said differential staining information comprises anti-CD4 label information in a first spatially registered image, and anti-CD3 label information in a second spatially registered image, said analyzing further comprising identifying target analytes that are present in the anti-CD4 label image and also present in the anti-CD3 label image, and further reporting the anti-CD4 positive/anti-CD3 positive target analytes as CD4 helper T cells.

Item 76. The method of Item 66, further comprising repeating steps (c) through (e) for multiple fields of view within said sample chamber.

Item 77. A device for analyzing a sample containing one or more target analytes, said device comprising a first substrate and a second substrate,
wherein said first substrate comprises a planar waveguide having a first outer surface and a first inner surface, and said second substrate comprises a second outer surface and a second inner surface, said first substrate and said second substrate being positioned such that at least a section of said first inner surface and a section of said second inner surface are apart from each other at a distance wherein said section of said first inner surface and said section of said second inner surface at least partly define a sample chamber for confining at least a portion of said sample,
said device further comprising a refractive volume for optically coupling one or more light beams provided by one or more light sources to the planar waveguide.

Item 78. The device of Item 77, wherein said refractive volume is configured for refracting the one or more light beams such that the at least one of the light beams is focused at the first inner surface of the planar waveguide at a non-zero, internal propagation angle relative to the planar surface for all light within the light beam.

Item 79. The device of Item 77, wherein said first inner surface is modified to create an attachment surface, said modification enhancing the immobilization of said target analytes.

Item 80. The device of Item 77, wherein said modification is performed by using a cationic polymer.

Changes may be made in the above methods and systems without departing from the scope hereof. It should thus be noted that the matter contained in the above description or shown in the accompanying drawings should be interpreted as illustrative and not in a limiting sense. The following claims are intended to cover generic and specific features described herein, as well as statements of the scope of the present method and system, which, as a matter of language, might be said to fall therebetween.

Although each of the aforedescribed embodiments have been illustrated with various components having particular respective orientations, it should be understood that the system as described in the present disclosure may take on a variety of specific configurations with the various components being located in a variety of positions and mutual orientations and still remain within the spirit and scope of the present disclosure. For example, it should be noted that the present configuration may be applicable for systems in which the core refractive index is greater than the refractive indices of the substrates, such as if a solid core material is used, as long as the surrounding medium refractive index is less than those of the substrates. Additionally, in the various figures described above, the gasket may be eliminated and replaced with direct laser welding of first and second substrates. Furthermore, suitable equivalents may be used in place of or in addition to the various components, the function and use of such substitute or additional components being held to be familiar to those skilled in the art and are therefore regarded as falling within the scope of the present disclosure. Therefore, the present examples are to be considered as illustrative and not restrictive, and the present disclosure is not to be limited to the details given herein but may be modified within the scope of the appended claims.

What is claimed is:

1. A system for analyzing a sample containing one or more target analytes, said system comprising a cartridge and a reader instrument,
said cartridge comprising a first substrate and a second substrate,
said first substrate comprising a planar waveguide, said planar waveguide having a first outer surface and a first inner surface,
said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample;
said reader instrument comprising
a receiving mechanism for positioning the cartridge therein, and
an imaging detector for detecting light signal from a field of view on or near the first inner surface of said planar waveguide;
said cartridge further comprising a refractive volume for optically coupling a light beam provided by a light source to the planar waveguide.

2. The system of claim 1, wherein said reader instrument further comprises a module for analyzing the light signal received by the imaging detector.

3. The system of claim 2, wherein said distance between said first inner surface and said second inner surface, together with said field of view having measurable dimensions defines a sample volume.

4. The system of claim 1, wherein said refractive volume is integrally formed from the planar waveguide.

5. The system of claim 1, wherein said refractive volume is configured for refracting the light beam such that the light beam is focused on the first inner surface of the planar waveguide at a non-zero, internal propagation angle relative to the first inner surface for all light within the light beam.

6. The system of claim 1, wherein said first inner surface is modified to create an attachment surface, said modification enhancing immobilization of said target analytes onto said first inner surface.

7. The system of claim 6, wherein said modification is performed by using a cationic polymer.

8. The system of claim 1, wherein said target analytes are labeled with one or more excitable tags.

9. The system of claim 1, wherein said target analytes are labeled with antibodies conjugated with a fluorophore.

10. The system of claim 3, wherein said analyzing by said module comprises enumerating said target analytes by counting the number of target analytes in at least one field of view captured by said imaging detector of said reader instrument.

11. The system of claim 10, wherein said number of target analytes in said at least one field of view represent total number of all target analytes in said sample volume, and concentration of said target analytes in said sample may be calculated by dividing the total number of target analytes obtained in claim 10 by the sample volume.

12. The system of claim 11, wherein said concentration is insensitive to the amount of the sample added to said sample chamber.

13. The system of claim 1, wherein the reader instrument further comprises one or more light sources for providing the light beam.

14. The system of claim 1, wherein at least a fraction of the light within said light beam is optically coupled to and contained between the first outer surface of the first substrate and the second outer surface of the second substrate, wherein said fraction of the light illuminates at least a portion of the sample confined within the sample chamber.

15. The system of claim 1, wherein said reader instrument further comprises an autofocus means.

16. The system of claim 1, wherein said reader instrument further comprises a light source and a condenser lens, said light source and condenser lens, in conjunction with said imaging detector, are capable of generating a brightfield microscopy image.

17. The system of claim 1, wherein said reader instrument comprises an actuation means for generating multiple fields of view by controlling relative position of said cartridge and said imaging detector.

18. The system of claim 17, wherein said actuation means controls relative position of said cartridge and said imaging detector by moving said cartridge relative to said imaging detector.

19. A device for analyzing a sample containing one or more target analytes, said device comprising a first substrate and a second substrate,
wherein said first substrate comprises a planar waveguide having a first outer surface and a first inner surface, and wherein said second substrate comprises a second outer surface and a second inner surface, said first substrate and said second substrate being positioned such that at least a section of said first inner surface and a section of said second inner surface are apart from each other at a distance wherein said section of said first inner surface and said section of said second inner surface at least partly define a sample chamber for confining at least a portion of said sample,
said device further comprising a refractive volume for optically coupling to the planar waveguide one or more light beams provided by one or more light sources.

20. The device of claim 19, wherein said refractive volume is configured for refracting the one or more light beams such that the at least one of the light beams is focused at the first inner surface of the planar waveguide at a non-zero, internal propagation angle relative to the planar surface for all light within the light beam.

21. The device of claim 19, wherein said first inner surface is modified to create an attachment surface, said modification enhancing the immobilization of said target analytes.

22. The device of claim 21, wherein said modification is performed by using a cationic polymer.

23. A method for analyzing a sample containing one or more target analytes, the method comprising:
(a) contacting said sample with a labeling molecule of a first type,
(b) introducing said sample prepared in step (a) into a cartridge, said cartridge comprising a first substrate, a second substrate, and a refractive volume,
said first substrate comprising a planar waveguide, said planar waveguide having a first outer surface and a first inner surface,
said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample, and
wherein said refractive volume optically couples light to the planar waveguide,
(c) positioning said cartridge in a reader instrument, wherein said instrument directs a light beam from a light source into said refractive volume such that the light beam is focused on the first inner surface of the planar waveguide at a non-zero, internal propagation angle relative to the first inner surface for all light within the light beam, wherein said light beam excites said labeling molecule of a first type bound to said one or more target analytes at said first inner surface of said planar waveguide;
(d) generating an image of a field of view having measurable dimensions at the first inner surface of said planar waveguide, said image resolving target analytes present in said sample.

24. A method for analyzing a sample containing one or more target analytes, the method comprising:
(a) contacting said sample with labeling molecules of a first type and labeling molecules of a second type;
(b) introducing said sample prepared in step (a) into a cartridge, said cartridge comprising a first substrate, a second substrate, and a refractive volume,
said first substrate comprising a planar waveguide, said planar waveguide having a first outer surface and a first inner surface,
said second substrate comprising a second outer surface and a second inner surface, wherein said first substrate and said second substrate are positioned such that at least a section of said first inner surface and a section of said second inner surface are apart from each other at a distance wherein said section of said first inner surface and said section of said second inner surface at least partly define a sample chamber for confining at least a portion of said sample, and
wherein said refractive volume optically couples light to the planar waveguide;
(c) positioning said cartridge in a reader instrument, wherein said instrument directs a first light beam from a first light source into said refractive volume, wherein said first light beam excites said labeling molecules of the first type, and wherein said instrument further directs a second light beam from a second light source into said refractive volume wherein said second light beam excites said labeling molecules of the second type;
(d) generating a first image and a second image of a single field of view having measurable dimensions at the first inner surface of said planar waveguide, wherein said images are spatially registered, and
(e) resolving target analytes present in the sample using said spatially registered images to generate differential labeling information.

25. The method of claim 24, wherein said first light beam and said second light beam are focused on the first inner surface of the planar waveguide at a non-zero, internal propagation angle relative to the planar surface for all light within the light beams.

26. The method of claim 24, wherein said target analytes in a field of view image represent all target analytes in the known sample volume defined by the said known distance between said first inner surface and said second inner surface, and the said field of view with known dimensions, thereby generating a concentration result in the form of number of target analytes per unit volume.

27. The method of claim 23, wherein the target analytes are CD4 helper T cells.

28. The method of claim 24, wherein the target analytes are CD4 helper T cells.
